SEQUENCE LISTING (153 sequences)

SEQ ID NO: 1 - protein, 25 aa, artificial sequence
  first 25 amino acid residues of the consensus sequence of pfam00229
KPAAHLVGKPLGQGPLSWENDGGTA

SEQ ID NO: 2 - protein, 11 aa, artificial sequence
  linker peptide CGG fused with AA 11-18 of mus musculus TNFalpha
CGGKPVAHVVA

SEQ ID NO: 3 - protein, 16 aa, artificial sequence
  linker peptide CGG fused with AA 9-20 of mus musculus TNFalpha
CGGSDKPVAHVVANHQ

SEQ ID NO: 4 - protein, 132 aa, Bacteriophage Q-beta
AKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAY

SEQ ID NO: 5 - protein, 329 aa, Bacteriophage Q-beta
MAKLETVTLGNIGKDGKQTLVLNPRGVNPTNGVASLSQAGAVPALEKRVTVSVSQPSRNRKNYKVQVKIQNPTACTANGSCDPSVTRQAYADVTFSFTQYSTDEERAFVRTELAALLASPLLIDAIDQLNPAYWTLLIAGGGSGSKPDPVIPDPPIDPPPGTGKYTCPFAIWSLEEVYEPPTKNRPWPIYNAVELQPREFDVALKDLLGNTKWRDWDSRLSYTTFRGCRGNGYIDLDATYLATDQAMRDQKYDIREGKKPGAFGNIERFIYLKSINAYCSLSDIAAYHADGVIVGFWRDPSSGGAIPFDFTKFDKTKCPIQAVIVVPRA

SEQ ID NO: 6 - protein, 129 aa, Bacteriophage R17
ASNFTQFVLVNDGGTGNVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 7 - protein, 130 aa, Bacteriophage fr
MASNFEEFVLVDNGGTGDVKVAPSNFANGVAEWISSNSRSQAYKVTCSVRQSSANNRKYTVKVEVPKVATQVQGGVELPVAAWRSYMNMELTIPVFATNDDCALIVKALQGTFKTGNPIATAIAANSGIY

SEQ ID NO: 8 - protein, 130 aa, Bacteriophage GA
MATLRSFVLVDNGGTGNVTVVPVSNANGVAEWLSNNSRSQAYRVTASYRASGADKRKYAIKLEVPKIVTQVVNGVELPGSAWKAYASIDLTIPIFAATDDVTVISKSLAGLFKVGNPIAEAISSQSGFYA

SEQ ID NO: 9 - protein, 132 aa, Bacteriophage SP
MAKLNQVTLSKIGKNGDQTLTLTPRGVNPTNGVASLSEAGAVPALEKRVTVSVAQPSRNRKNFKVQIKLQNPTACTRDACDPSVTRSAFADVTLSFTSYSTDEERALIRTELAALLADPLIVDAIDNLNPAY

SEQ ID NO: 10 - protein, 329 aa, Bacteriophage SP
AKLNQVTLSKIGKNGDQTLTLTPRGVNPTNGVASLSEAGAVPALEKRVTVSVAQPSRNRKNFKVQIKLQNPTACTRDACDPSVTRSAFADVTLSFTSYSTDEERALIRTELAALLADPLIVDAIDNLNPAYWAALLVASSGGGDNPSDPDVPVVPDVKPPDGTGRYKCPFACYRLGSIYEVGKEGSPDIYERGDEVSVTFDYALEDFLGNTNWRNWDQRLSDYDIANRRRCRGNGYIDLDATAMQSDDFVLSGRYGVRKVKFPGAFGSIKYLLNIQGDAWLDLSEVTAYRSYGMVIGFWTDSKSPQLPTDFTQFNSANCPVQTVIIIPS

SEQ ID NO: 11 - protein, 130 aa, Bacteriophage MS2
MASNFTQFVLVDNGGTGDVTVAPSNFANGVAEWISSNSRSQAYKVTCSVRQSSAQNRKYTIKVEVPKVATQTVGGVELPVAAWRSYLNMELTIPIFATNSDCELIVKAMQGLLKDGNPIPSAIAANSGIY

SEQ ID NO: 12 - protein, 133 aa, Bacteriophage M11
MAKLQAITLSGIGKKGDVTLDLNPRGVNPTNGVAALSEAGAVPALEKRVTISVSQPSRNRKNYKVQVKIQNPTSCTASGTCDPSVTRSAYSDVTFSFTQYSTVEERALVRTELQALLADPMLVNAIDNLNPAY

SEQ ID NO: 13 - protein, 133 aa, Bacteriophage MX1
MAKLQAITLSGIGKNGDVTLNLNPRGVNPTNGVAALSEAGAVPALEKRVTISVSQPSRNRKNYKVQVKIQNPTSCTASGTCDPSVTRSAYADVTFSFTQYSTDEERALVRTELKALLADPMLIDAIDNLNPAY

SEQ ID NO: 14 - protein, 330 aa, Bacteriophage NL95
MAKLNKVTLTGIGKAGNQTLTLTPRGVNPTNGVASLSEAGAVPALEKRVTVSVAQPSRNRKNYKVQIKLQNPTACTKDACDPSVTRSGSRDVTLSFTSYSTERERALIRTELAALLKDDLIVDAIDNLNPAYWAALLAASPGGGNNPYPGVPDSPNVKPPGGTGTYRCPFACYRRGELITEAKDGACALYACGSEALVEFEYALEDFLGNEFWRNWDGRLSKYDIETHRRCRGNGYVDLDASVMQSDEYVLSGAYDVVKMQPPGTFDSPRYYLHLMDGIYVDLAEVTAYRSYGMVIGFWTDSKSPQLPTDFTRFNRHNCPVQTVIVIPSL

SEQ ID NO: 15 - protein, 129 aa, Bacteriophage f2
ASNFTQFVLVNDGGTGNVTVAPSNFANGVAEWISSNSRSQAYKVTCSVRQSSAQNRKYTIKVEVPKVATQTVGGVELPVAAWRSYLNLELTIPIFATNSDCELIVKAMQGLLKDGNPIPSAIAANSGIY

SEQ ID NO: 16 - protein, 128 aa, Bacteriophage PP7
MSKTIVLSVGEATRTLTEIQSTADRQIFEEKVGPLVGRLRLTASLRQNGAKTAYRVNLKLDQADVVDCSTSVCGELPKVRYTQVWSHDVTIVANSTEASRKSLYDLTKSLVATSQVEDLVVNLVPLGR

SEQ ID NO: 17 - protein, 132 aa, Artificial Sequence
  Bacteriophage Qbeta 240 mutant
AKLETVTLGNIGRDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 18 - protein, 132 aa, Artificial Sequence
  Bacteriophage Q-beta 243 mutant
AKLETVTLGKIGKDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 19 - protein, 132 aa, Artificial Sequence
  Bacteriophage Q-beta 250 mutant
ARLETVTLGNIGRDGKQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 20 - protein, 132 aa, Artificial Sequence
  Bacteriophage Q-beta 251 mutant
AKLETVTLGNIGKDGRQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 21 - protein, 132 aa, Artificial Sequence
  Bacteriophage Q-beta 259 mutant
ARLETVTLGNIGKDGRQTLVLNPRGVNPTNGVASLSQAGAVPALEKRVTVSVSQPSRNRKNYKVQVKIQNPTACTANGSCDPSVTRQKYADVTFSFTQYSTDEERAFVRTELAALLASPLLIDAIDQLNPAY

SEQ ID NO: 22 - protein, 12 aa, artificial sequence
  linker peptide CGG fused to AA 162 to 170 of mus musculus RANKL
CGGQPFAHLTIN

SEQ ID NO: 23 - protein, 15 aa, artificial sequence
  linker peptide CGG fused to AA 160 to 171 of mus musculus RANKL
CGGEAQPFAHLTINA

SEQ ID NO: 24 - protein, 4 aa, Artificial Sequence
  C.terminal linker
GGCG

SEQ ID NO: 25 - protein, 185 aa, Hepatitis B virus
MDIDPYKEFGATVELLSFLPSDFFPSVRDLLDTASALYREALESPEHCSPHHTALRQAILCWGELMTLATWVGNNLEDPASRDLVVNYVNTNMGLKIRQLLWFHISCLTFGRETVLEYLVSFGVWIRTPPAYRPPNAPILSTLPETTVVRRRDRGRSPRRRTPSPRRRRSQSPRRRRSQSRESQC

SEQ ID NO: 26 - protein, 152 aa, Hepatitis B virus
MDIDPYKEFGATVELLSFLPSDFFPSVRDLLDTAAALYRDALESPEHCSPHHTALRQAILCWGDLMTLATWVGTNLEDGGKGGSRDLVVSYVNTNVGLKFRQLLWFHISCLTFGRETVLEYLVSFGVWIRTPPAYRPPNAPILSTLPETTVV

SEQ ID NO: 27 - protein, 5 aa, Artificial Sequence
  Linker
GGKGG

SEQ ID NO: 28 - protein, 131 aa, Bacteriophage AP205
MANKPMQPITSTANKIVWSDPTRLSTTFSASLLRQRVKVGIAELNNVSGQYVSVYKRPAPKPEGCADACVIMPNENQSIRTVISGSAENLATLKAEWETHKRNVDTLFASGNAGLGFLDPTAAIVSSDTTA

SEQ ID NO: 29 - protein, 23 aa, artificial sequence
  linker peptide CGG fused with AA 4-23 from muc musculus TNFalpha
CGGSSQNSSDKPVAHVVANHQVE

SEQ ID NO: 30 - protein, 23 aa, artificial sequence
  linker peptide CGG fused to AA 155-174 of mus musculus RANKL
CGGQRGKPEAQPFAHLTINAASI

SEQ ID NO: 31 - protein, 6 aa, artificial sequence
  peptide from TNFalpha
VAHVVA

SEQ ID NO: 32 - protein, 8 aa, artificial sequence
  peptide
KPVAHVVA

SEQ ID NO: 33 - protein, 9 aa, artificial sequence
  peptide
KPVAHVVAN

SEQ ID NO: 34 - protein, 6 aa, artificial sequence
  peptide
AAHLVG

SEQ ID NO: 35 - protein, 6 aa, artificial sequence
  peptide
AAHLIG

SEQ ID NO: 36 - protein, 8 aa, artificial sequence
  peptide
KPAAHLVG

SEQ ID NO: 37 - protein, 8 aa, artificial sequence
  peptide
KPAAHLIG

SEQ ID NO: 38 - protein, 9 aa, artificial sequence
  peptide
LKPAAHLVG

SEQ ID NO: 39 - protein, 9 aa, artificial sequence
  peptide
LKPAAHLIG

SEQ ID NO: 40 - protein, 6 aa, artificial sequence
  peptide
AAHLIG

SEQ ID NO: 41 - protein, 8 aa, artificial sequence
  peptide
PAAHLIGA

SEQ ID NO: 42 - protein, 8 aa, artificial sequence
  peptide
PAAHLIGI

SEQ ID NO: 43 - protein, 6 aa, artificial sequence
  peptide
AAHVIS

SEQ ID NO: 44 - protein, 6 aa, artificial sequence
  peptide
AAHVVS

SEQ ID NO: 45 - protein, 8 aa, artificial sequence
  peptide
QIAAHVIS

SEQ ID NO: 46 - protein, 8 aa, artificial sequence
  peptide
RIAAHVIS

SEQ ID NO: 47 - protein, 10 aa, artificial sequence
  peptide
NPQIAAHVIS

SEQ ID NO: 48 - protein, 10 aa, artificial sequence
  peptide
DPQIAAHVIS

SEQ ID NO: 49 - protein, 10 aa, artificial sequence
  peptide
DPQIAAHVVS

SEQ ID NO: 50 - protein, 10 aa, artificial sequence
  peptide
EPQIAAHVIS

SEQ ID NO: 51 - protein, 6 aa, artificial sequence
  peptide
VAHLTG

SEQ ID NO: 52 - protein, 8 aa, artificial sequence
  peptide
RSVAHLTG

SEQ ID NO: 53 - protein, 8 aa, artificial sequence
  peptide
RKVAHLTG

SEQ ID NO: 54 - protein, 8 aa, artificial sequence
  peptide
RRAAHLTG

SEQ ID NO: 55 - protein, 8 aa, artificial sequence
  peptide
KKAAHLTG

SEQ ID NO: 56 - protein, 6 aa, artificial sequence
  peptide
AELQLN

SEQ ID NO: 57 - protein, 6 aa, artificial sequence
  peptide
LQLNLT

SEQ ID NO: 58 - protein, 6 aa, artificial sequence
  peptide
LQLNHT

SEQ ID NO: 59 - protein, 7 aa, artificial sequence
  peptide
VAELQLN

SEQ ID NO: 60 - protein, 7 aa, artificial sequence
  peptide
TAELQLN

SEQ ID NO: 61 - protein, 8 aa, artificial sequence
  peptide
TAELQLNL

SEQ ID NO: 62 - protein, 8 aa, artificial sequence
  peptide
VAELQLNL

SEQ ID NO: 63 - protein, 8 aa, artificial sequence
  peptide
VAELQLNH

SEQ ID NO: 64 - protein, 5 aa, artificial sequence
  peptide
AAHIT

SEQ ID NO: 65 - protein, 5 aa, artificial sequence
  peptide
AAHLT

SEQ ID NO: 66 - protein, 7 aa, artificial sequence
  peptide
VAAHITG

SEQ ID NO: 67 - protein, 10 aa, artificial sequence
  peptide
PQKVAAHITG

SEQ ID NO: 68 - protein, 10 aa, artificial sequence
  peptide
PQRVAAHITG

SEQ ID NO: 69 - protein, 6 aa, artificial sequence
  peptide
FAHLTI

SEQ ID NO: 70 - protein, 6 aa, artificial sequence
  peptide
SAHLTV

SEQ ID NO: 71 - protein, 10 aa, artificial sequence
  peptide
EAQPFAHLTI

SEQ ID NO: 72 - protein, 9 aa, artificial sequence
  peptide
QPFAHLTIN

SEQ ID NO: 73 - protein, 14 aa, artificial sequence
  peptide
KPEAQPFAHLTINA

SEQ ID NO: 74 - protein, 14 aa, artificial sequence
  peptide
KLEAQPFAHLTINA

SEQ ID NO: 75 - protein, 20 aa, artificial sequence
  peptide
KRSKLEAQPFAHLTINATDI

SEQ ID NO: 76 - protein, 20 aa, artificial sequence
  peptide
QRGKPEAQPFAHLTINAASI

SEQ ID NO: 77 - protein, 6 aa, artificial sequence
  peptide
AAHYEV

SEQ ID NO: 78 - protein, 9 aa, artificial sequence
  peptide
RAIAAHYEV

SEQ ID NO: 79 - protein, 8 aa, artificial sequence
  peptide
AAHYEVHP

SEQ ID NO: 80 - protein, 13 aa, artificial sequence
  peptide
ARRAIAAHYEVHP

SEQ ID NO: 81 - protein, 13 aa, artificial sequence
  peptide
PRRAIAAHYEVHP

SEQ ID NO: 82 - protein, 6 aa, artificial sequence
  peptide
SVLHLV

SEQ ID NO: 83 - protein, 8 aa, artificial sequence
  peptide
HSVLHLVP

SEQ ID NO: 84 - protein, 8 aa, artificial sequence
  peptide
QSVLHLVP

SEQ ID NO: 85 - protein, 11 aa, artificial sequence
  peptide
KKQHSVLHLVP

SEQ ID NO: 86 - protein, 11 aa, artificial sequence
  peptide
KKKHSVLHLVP

SEQ ID NO: 87 - protein, 11 aa, artificial sequence
  peptide
KKKQSVLHLVP

SEQ ID NO: 88 - protein, 6 aa, artificial sequence
  peptide
LQLIAD

SEQ ID NO: 89 - protein, 10 aa, artificial sequence
  peptide
QDCLQLIADS

SEQ ID NO: 90 - protein, 10 aa, artificial sequence
  peptide
QACLQLIADS

SEQ ID NO: 91 - protein, 6 aa, artificial sequence
  peptide
AAHLTG

SEQ ID NO: 92 - protein, 8 aa, artificial sequence
  peptide
NPAAHLTG

SEQ ID NO: 93 - protein, 8 aa, artificial sequence
  peptide
AAHLTGAN

SEQ ID NO: 94 - protein, 12 aa, artificial sequence
  peptide
VNPAAHLTGANS

SEQ ID NO: 95 - protein, 12 aa, artificial sequence
  peptide
ANPAAHLTGANA

SEQ ID NO: 96 - protein, 6 aa, artificial sequence
  peptide
RAHLTV

SEQ ID NO: 97 - protein, 6 aa, artificial sequence
  peptide
RAHLTI

SEQ ID NO: 98 - protein, 6 aa, artificial sequence
  peptide
KAHLTI

SEQ ID NO: 99 - protein, 6 aa, artificial sequence
  peptide
TQHFKN

SEQ ID NO: 100 - protein, 6 aa, artificial sequence
  peptide
AVVHLQ

SEQ ID NO: 101 - protein, 6 aa, artificial sequence
  peptide
VVHLQG

SEQ ID NO: 102 - protein, 9 aa, artificial sequence
  peptide
QPAVVHLQG

SEQ ID NO: 103 - protein, 10 aa, artificial sequence
  peptide
PAVVHLQGQG

SEQ ID NO: 104 - protein, 12 aa, artificial sequence
  peptide
TRENQPAVVHLQ

SEQ ID NO: 105 - protein, 14 aa, artificial sequence
  peptide
ENQPAVVHLQGQGS

SEQ ID NO: 106 - protein, 14 aa, artificial sequence
  peptide
QPAVVHLQGQGSAI

SEQ ID NO: 107 - protein, 5 aa, artificial sequence
  peptide
CMVKF

SEQ ID NO: 108 - protein, 5 aa, artificial sequence
  peptide
CMAKF

SEQ ID NO: 109 - protein, 8 aa, artificial sequence
  peptide
ESCMVKFE

SEQ ID NO: 110 - protein, 8 aa, artificial sequence
  peptide
EPCMAKFG

SEQ ID NO: 111 - protein, 6 aa, artificial sequence
  peptide
WAYLQV

SEQ ID NO: 112 - protein, 6 aa, artificial sequence
  peptide
AAYMRV

SEQ ID NO: 113 - protein, 9 aa, artificial sequence
  peptide
KGAAAYMRV

SEQ ID NO: 114 - protein, 9 aa, artificial sequence
  peptide
KKSWAYLQV

SEQ ID NO: 115 - protein, 6 aa, artificial sequence
  peptide
FAQLVA

SEQ ID NO: 116 - protein, 6 aa, artificial sequence
  peptide
FAKLLA

SEQ ID NO: 117 - protein, 8 aa, artificial sequence
  peptide
LVAQNVLL

SEQ ID NO: 118 - protein, 8 aa, artificial sequence
  peptide
LLAKNQAS

SEQ ID NO: 119 - protein, 9 aa, artificial sequence
  peptide
QGMFAQLVA

SEQ ID NO: 120 - protein, 6 aa, artificial sequence
  peptide
FILTSQ

SEQ ID NO: 121 - protein, 6 aa, artificial sequence
  peptide
FIGTSK

SEQ ID NO: 122 - protein, 6 aa, artificial sequence
  peptide
FILPLQ

SEQ ID NO: 123 - protein, 9 aa, artificial sequence
  peptide
KGFILTSQK

SEQ ID NO: 124 - protein, 9 aa, artificial sequence
  peptide
RLFIGTSKK

SEQ ID NO: 125 - protein, 10 aa, artificial sequence
  peptide
AQPFAHLTIN

SEQ ID NO: 126 - protein, 20 aa, artificial sequence
  peptide
RKTRARRAIAAHYEVHPRPG

SEQ ID NO: 127 - protein, 10 aa, artificial sequence
  peptide
SKPVAHVVAN

SEQ ID NO: 128 - protein, 20 aa, artificial sequence
  peptide
QRGDEDPQIAAHVVSEANSN

SEQ ID NO: 129 - protein, 20 aa, artificial sequence
  peptide
NLRNIIQDSLQLIADSDTPT

SEQ ID NO: 130 - protein, 20 aa, artificial sequence
  peptide
ETDLNPELPAAHLIGAWMSG

SEQ ID NO: 131 - protein, 20 aa, artificial sequence
  Peptide
ETDLSPGLPAAHLIGAPLKG

SEQ ID NO: 132 - protein, 20 aa, artificial sequence
  Peptide
HLAHSTLKPAAHLIGDPSKQ

SEQ ID NO: 133 - protein, 20 aa, artificial sequence
  Peptide
QKGDQNPQIAAHVISEASSK

SEQ ID NO: 134 - protein, 20 aa, artificial sequence
  Peptide
PPEKKELRKVAHLTGKSNSR

SEQ ID NO: 135 - protein, 20 aa, artificial sequence
  Peptide
LGWDVAELQLNHTGPQQDPR

SEQ ID NO: 136 - protein, 18 aa, artificial sequence
  Peptide
ERGPQRVAAHITGTRGRS

SEQ ID NO: 137 - protein, 19 aa, artificial sequence
  Peptide
QKQKKQHSVLHLVPINATS

SEQ ID NO: 138 - protein, 16 aa, artificial sequence
  Peptide
VTQDCLQLIADSETPT

SEQ ID NO: 139 - protein, 20 aa, artificial sequence
  Peptide
ERRSHEVNPAAHLTGANSSL

SEQ ID NO: 140 - protein, 20 aa, artificial sequence
  Peptide
PLRADGDKPRAHLTVVRQTP

SEQ ID NO: 141 - protein, 20 aa, artificial sequence
  Peptide
AGTRENQPAVVHLQGQGSAI

SEQ ID NO: 142 - protein, 20 aa, artificial sequence
  Peptide
QLETAKEPCMAKFGPLPSKW

SEQ ID NO: 143 - protein, 20 aa, artificial sequence
  Peptide
LKRAPFKKSWAYLQVAKHLN

SEQ ID NO: 144 - protein, 17 aa, artificial sequence
  Peptide
DLRQGMFAQLVAQNVLL

SEQ ID NO: 145 - protein, 19 aa, artificial sequence
  Peptide
FTEYKKEKGFILTSQKEDE

SEQ ID NO: 146 - protein, 13 aa, artificial sequence
  Peptide
CGGSKPVAHVVAN

SEQ ID NO: 147 - protein, 13 aa, artificial sequence
  Peptide
CGGAQPFAHLTIN

SEQ ID NO: 148 - protein, 23 aa, artificial sequence
  Peptide
CGGKRSKLEAQPFAHLTINATDI

SEQ ID NO: 149 - protein, 12 aa, artificial sequence
  Peptide
EAQPFAHLTINA

SEQ ID NO: 150 - protein, 23 aa, artificial sequence
  Peptide
CGGQRGDEDPQIAAHVVSEANSN

SEQ ID NO: 151 - protein, 23 aa, artificial sequence
  Paptide
CGGNLRNIIQDSLQLIADSDTPT

SEQ ID NO: 152 - protein, 23 aa, artificial sequence
  Peptide
CGGETDLNPELPAAHLIGAWMSG

SEQ ID NO: 153 - protein, 13 aa, artificial sequence
  Peptide
SDKPVAHVVANHQ

(19) United States
(12) Patent Application Publication
(10) Pub. No.: US 2008/0019991 A1
(43) Pub. Date: Jan. 24, 2008

(54) Carrier Conjugates Of Tnf-Peptides

(75) Inventors: Martin F. Bachmann, Seuzach (CH); Patrik Maurer, Winterthur (CH); Gunther Spohn, Zurich (CH)

Correspondence Address: STERNE, KESSLER, GOLDSTEIN & FOX P.L.L.C., 1100 NEW YORK AVENUE, N.W., WASHINGTON, DC, 20005, US

(73) Assignee: Cytos Biotechnology AG, Zurich-Schlieren (CH)

(21) Appl. No.: 11/628,243

(22) PCT Filed: Jun. 2, 2005
(86) PCT No.: PCT/EP05/05936

§ 371 Date: Dec. 1, 2006

Related U.S. Application Data
(60) Provisional application No. 60/575,821, filed on Jun. 2, 2004.

(51) Int. Cl. A61K 39/00; A61K 45/00; C12N 7/01

(52) U.S. Cl. 424/185.1; 424/93.6; 435/235.1

(57) ABSTRACT

The present invention is related to the fields of molecular biology, virology, immunology and medicine. The invention provides a modified virus-like particle (VLP) comprising a VLP and a particular peptide derived from a polypeptide from the TNF-superfamily linked thereto. The invention also provides a process for producing the modified VLP. The modified VLPs of the invention are useful in the production of vaccines for the treatment of autoimmune diseases and bone-related diseases and to efficiently induce immune responses, in particular antibody responses. Furthermore, the compositions of the invention are particularly useful to efficiently induce self-specific immune responses within the indicated context.

### BACKGROUND OF THE INVENTION

1. Field of the Invention

The present invention is related to the fields of molecular biology, virology, immunology and medicine. The invention provides, inter alia, a modified virus-like particle (VLP) comprising: a VLP and at least one particular peptide derived from a polypeptide from the TNF-superfamily linked thereto. The invention also provides a process for producing the modified VLP. The modified VLPs of the invention are useful in the production of vaccines for the treatment of autoimmune diseases and bone-related diseases and to efficiently induce immune responses, in particular antibody responses. Furthermore, the compositions of the invention are particularly useful to efficiently induce self-specific immune responses within the indicated context.

2. Related Art

Members of the tumor necrosis factor (TNF) family play key roles in the development and function of the immune system (F. Mackay and S. L. Kalled, Current Opinion in Immunology, 14: 783-790 (2002)). The vast majority of these members are powerful modulators of critical immune functions and participate in pathogenic mechanisms leading to autoimmune disease. For example, altered regulation of TNFα may contribute to a breakdown in immune tolerance and the development of autoimmune disease, whereas, for example, RANKL has emerged with novel functions that regulate both T and B cell immune tolerance and participate in tissue destruction in autoimmunity (F. Mackay and S. L. Kalled, Current Opinion in Immunology, 14: 783-790 (2002)).

It is usually difficult to induce antibody responses against self-antigens. One way to improve the efficiency of vaccination is to increase the degree of repetitiveness of the antigen applied. Unlike isolated proteins, viruses induce prompt and efficient immune responses in the absence of any adjuvant both with and without T-cell help (Bachmann and Zinkernagel, Ann. Rev. Immunol: 15:235-270 (1991)). Although viruses often consist of few proteins, they are able to trigger much stronger immune responses than their isolated components. For B-cell responses, it is known that one crucial factor for the immunogenicity of viruses is the repetitiveness and order of surface epitopes. Many viruses exhibit a quasi-crystalline surface that displays a regular array of epitopes which efficiently crosslinks epitope-specific immunoglobulins on B-cells (Bachmann and Zinkemagel, Immunol. Today 17:553-558 (1996)). This crosslinking of surface immunoglobulins on B cells is a strong activation signal that directly induces cell-cycle progression and the production of IgM antibodies. Further, such triggered B-cells are able to activate T helper cells, which in turn induce a switch from IgM to IgG antibody production in B cells and the generation of long-lived B cell memory—the goal of any vaccination (Bachmann and Zinkernagel, Ann. Rev. Immunol. 15:235-270 (1997)). Viral structure is even linked to the generation of anti-antibodies in autoimmune disease and as a part of the natural response to pathogens (see Fehr, T., et al, J Exp. Med. 185:1785-1792 (1997)). Thus, antigens presented by a highly organized viral surface are able to induce strong antibody responses against the antigens.

As indicated, however, the immune system usually fails to produce antibodies against self-derived structures. For soluble antigens present at low concentrations, this is due to tolerance at the Th-cell level. Under these conditions, coupling the self-antigen to a carrier that can deliver T help may break tolerance. For soluble proteins present at high concentrations or membrane proteins at low concentration, B- and Th-cells may be tolerant. However, B-cell tolerance may be reversible (anergy) and can be broken by administration of the antigen in a highly organized fashion coupled to a foreign carrier (Bachmann and Zinkemagel, Ann. Rev. Immunol. 15:235-270 (1997)).

Recently methods for vaccinations against self-antigens derived from the TNF family have been disclosed, e.g. in, WO 00/23955, WO 02/056905 and WO 03/039225. The vaccines disclosed in these patent applications contain carrier proteins, in particular virus-like particles (VLPs), to which self-antigens derived from TNFα LTα, LTβ, and RANKL are attached. Typically, these prior art vaccines contain the protein form of the corresponding member of the TNF-superfamily to generate strong antibody responses against the protein form.

### BRIEF SUMMARY OF THE INVENTION

We have found that TNF peptides of the invention derived from the N-terminal region of a TNF-like domain of a member of the TNF-superfamily and coupled to VLPs were able to induce strong antibody responses against the protein form of that same member of the TNF-superfamily. We have identified a short epitope, which is conserved in the whole TNF-superfamily and which is useful for vaccination against TNF-superfamily-members surprisingly providing a route for the treatment of several disorders and diseases in which members of the TNF-superfamily are involved, among them autoimmune diseases and/or bone-related diseases. Thus, antibodies directed against a certain N-terminal region of a TNF-like domain of one TNF-superfamily member are, unexpectedly, effective against the respective member of the TNF-superfamily. The present invention thus provides a prophylactic and therapeutic means for the treatment of autoimmune and/or bone-related diseases, which is based on administration of particular TNF-superfamily-member-derived peptides bound to a core particle, in particular on a VLP-TNF-superfamily-member-derived-peptide-conjugate and particularly on an ordered and repetitive array. The TNF-superfamily-member-derived-peptide of the invention comprises a peptide sequence homologous to or identical with amino acid residues 3 to 8 of the consensus sequence for the conserved domain pfam 00229 (SEQ ID NO:1). These prophylactic and therapeutic compositions are able to induce high titers of anti-TNF-superfamily-member antibodies in a vaccinated animal or human. As indicated, TNF-superfamily-member-derived-peptide coupled to a core particle can be used, when, and alternatively administered together with or without adjuvant, to induce TNF-superfamily-member-specific antibodies in humans and in animals.

Therefore, TNF-superfamily-member-derived peptides, coupled either C- or N-terminally to a core particle, preferably to a virus-like particle (VLP), are capable of inducing highly specific anti-TNF-superfamily-member antibodies typically being capable of neutralizing the function of a TNF-superfamily-member before it continues to exert an unwanted effect in a disease or disorder related situation.

We have found that antibodies generated from vaccination with C- or N-terminally linked TNF-superfamily-member-derived-peptide of the invention to a core particle or, preferably to a VLP, are able to bind to the respective human TNF-superfamily-member. Therefore, the present invention focuses on vaccination strategies against a TNF-superfamily-member involved in disease as a treatment for autoimmune-diseases and/or bone-related diseases.

As shown herein, and in particular in Example 1 and 4 vaccination with C- or N-terminally linked TNFα-peptide of the invention, and in particular N-terminally linked TNFα-peptide, to a core particle or, preferably to a VLP, leads to the induction of antibodies which also are able to bind to the protein form of TNFα. Likewise, as shown in particular in Example 5, vaccination with C- or N-terminally linked RANKL-peptide, and in particular N-terminally linked RANKL-peptide, to a core particle or, preferably to a VLP, leads to the induction of antibodies which also are able to bind to the protein form of RANKL. Antibodies that target TNFα and RANKL, respectively, are potential therapeutics for autoimmune-diseases and/or bone-related diseases, respectively.

In a preferred embodiment of the present invention, the TNF-peptides of the invention consists of a peptide with a length of 4, 5 or 6 to 50 amino acid residues, preferably with a length of from 4, 5 or 6 to 40 amino acid residues, more preferably with a length of from 4, 5 or 6 to 30 amino acid residues, even more preferably with a length of from 4 to 20 amino acid residues, again even more preferably with a length of from 4, 5 or 6 to 18 amino acid residues and even more preferred with a length of from 4, 5 or 6 to 16 amino acid residues. Vaccination against self-antigens, such as the members of the TNF superfamily, by using the protein form may lead to undesired inflammatory and/or cytotoxic immune responses. Therefore, vaccination using shorter peptide fragments is typically preferred.

The present invention, thus, also provides a composition comprising (a) a core particle with at least one first attachment site; and (b) at least one antigen or antigenic determinant with at least one second attachment site, wherein said antigen or antigenic determinant is a TNF-superfamily-derived-peptide (hereinafter called TNF-peptide) of the invention, and wherein said second attachment site being selected from the group consisting of (i) an attachment site not naturally occurring with said antigen or antigenic determinant; and (ii) an attachment site naturally occurring with said antigen or antigenic determinant, wherein said second attachment site is capable of association to said first attachment site; and wherein said antigen or antigenic determinant and said core particle interact through said association, preferably to form an ordered and repetitive antigen array. Preferred embodiments of core particles suitable for use in the present invention are a virus, a virus-like particle (VLP), a bacteriophage, a virus-like particle of a RNA-phage, a bacterial pilus or flagella or any other core particle having an inherent repetitive structure, preferably such a repetitive structure which is capable of forming an ordered and repetitive antigen array in accordance with the present invention.

More specifically, the invention provides a modified VLP comprising a virus-like particle and at least one TNF-peptide of the invention bound thereto. The invention also provides a process for producing the modified VLPs of the invention. The modified VLPs and compositions of the invention are useful in the production of vaccines for the treatment of autoimmune-diseases and of bone-related diseases and as a pharmaceutical to prevent or cure autoimmune-diseases and of bone-related diseases, also to efficiently induce immune responses, in particular antibody responses. Furthermore, the modified VLPs and compositions of the invention are particularly useful to efficiently induce self-specific immune responses within the indicated context.

In the present invention, a TNF-peptide of the invention is bound to a core particle and VLP, respectively, preferably in an oriented manner, preferably yielding an ordered and repetitive TNF-peptide antigen array. Furthermore, the highly repetitive and organized structure of the core particles and VLPs, respectively, can mediate the display of the TNF-peptide in a highly ordered and repetitive fashion leading to a highly organized and repetitive antigen array. Furthermore, binding of the TNF-peptide of the invention to the core particle and VLP, respectively, without being bound to any theory, may function by providing T helper cell epitopes, since the core particle or the VLP is foreign to the host immunized with the core particle-TNF-peptide array and VLP-TNF-peptide array, respectively. Preferred arrays differ from prior art conjugates, in particular, in their highly organized structure, dimensions, and in the repetitiveness of the antigen on the surface of the array.

In one aspect of the invention, the TNF-peptide of the invention is expressed in a suitable expression host, or synthesized, while the core particle and the VLP, respectively, is expressed and purified from an expression host suitable for the folding and assembly of the core particle and the VLP, respectively. TNF-peptides of the invention may be chemically synthesized. The TNF-peptide-array of the invention is then assembled by binding the TNF-peptide of the invention to the core particle and the VLP, respectively.

In a preferred embodiment, the present invention provides for a modified VLP comprising (a) a virus-like particle, and (b) at least one TNF-peptide of the invention, and wherein said TNF-peptide of the invention is linked to said virus-like particle.

In another aspect, the present invention provides a modified virus like particle (VLP) comprising (a) a virus like particle (VLP), and (b) at least one peptide (TNF-peptide) comprising a peptide sequence homologous to amino acid residues 3 to 8 of the consensus sequence for the conserved domain pfam 00229 (SEQ ID NO:1), preferably a peptide sequence homologous to amino acid residues 1 to 8 of the consensus sequence for the conserved domain pfam 00229 (SEQ ID NO:1), wherein a) and b) are linked with one another, and wherein said TNF-peptide consists of a peptide with a length of 4, 5 or 6 to 18 amino acid residues, preferably with a length of 4, 5 or 6 to 16 amino acid residues, more preferably with a length of 4, 5 or 6 to 14 amino acid residues, when the TNF-peptide is a peptide from human or mouse TNFα, and wherein TNF-peptide consists of a peptide with a length of 4, 5 or 6 to 50 amino acid residues, preferably with a length of 4, 5 or 6 to 40 amino acid residues, more preferably with a length of 4, 5 or 6 to 30 amino acid residues, when the TNF-peptide is a peptide from human or mouse RANKL, from human or mouse LTα, or from human or mouse LTβ, or from human or mouse LTα/LTβ.

In a further aspect, the present invention provides a composition and also a pharmaceutical composition comprising (a) the modified core particle, and in case of the pharmaceutical composition, in particular a modified VLP, and (b) an acceptable pharmaceutical carrier.

In a further aspect, the present invention provides for a pharmaceutical composition, preferably a vaccine composition, comprising (a) a virus-like particle; and (b) at least one TNF-peptide of the invention; and wherein said TNF-peptide of the invention is linked to said virus-like particle.

In still a further aspect, the present invention provides for a process for producing a modified VLP of the invention comprising (a) providing a virus-like particle; and (b) providing at least one TNF-peptide of the invention; (c) combining said virus-like particle and said TNF-peptide of the invention so that said TNF-peptide is bound to said virus-like particle, in particular under conditions suitable for mediating a link between the VLP and the TNF-peptide.

Analogously, the present invention provides a process for producing a modified core particle of the invention comprising: (a) providing a core particle with at least one first attachment site; (b) providing at least one TNF-peptide of the invention with at least one second attachment site, wherein said second attachment site being selected from the group consisting of (i) an attachment site not naturally occurring with said TNF-peptide of the invention; and (ii) an attachment site naturally occurring within said TNF-peptide of the invention; and wherein said second attachment site is capable of association to said first attachment site; and (c) combining said core particle and said at least one TNF-peptide of the invention, wherein said TNF-peptide of the invention and said core particle interact through said association, preferably to form an ordered and repetitive antigen array.

In another aspect, the present invention provides for a method of immunization comprising administering the modified VLP, the composition or pharmaceutical composition of the invention, or the vaccine composition to an animal or human, preferably a human.

In again another aspect, the present invention provides for a method of treating an autoimmune disease or a bone related disease by administering to a subject, preferably to a human, the modified VLP, the composition, the pharmaceutical composition or the vaccine composition of the invention, wherein preferably the autoimmune disease or the bone related disease is selected from the group consisting of (a) psoriasis; (b) rheumatoid arthritis; (c) multiple sclerosis; (d) diabetes; (e) osteoporosis; (f) ankylosing spondylitis; (g) atherosclerosis; (h) autoimmune hepatitis; (i) autoimmune thyroid disease; (j) bone cancer pain; (k) bone metastasis; (l) inflammatory bowel disease; (m) multiple myeloma; (n) myasthenia gravis; (O) myocarditis; (p) Paget's disease; (q) periodontal disease; (r) periodontitis; (s) periprosthetic osteolysis; (t) polymyositis; (u) primary biliary cirrhosis; (v) psoriatic arthritis; (w) Sjögren's syndrome; (x) Still's disease; (y) systemic lupus erythematosus; and (z) vasculitis.

In a further aspect, the present invention provides for a use of the modified VLP, the composition, the pharmaceutical composition or the vaccine composition of the invention for the manufacture of a medicament for treatment of autoimmune-diseases and/or of bone-related diseases, wherein preferably the autoimmune disease or the bone related disease is selected from the group consisting of (a) psoriasis; (b) rheumatoid arthritis; (c) multiple sclerosis; (d) diabetes; (e) osteoporosis; (f) ankylosing spondylitis; (g) atherosclerosis; (h) autoimmune hepatitis; (i) autoimmune thyroid disease; (j) bone cancer pain; (k) bone metastasis; (l) inflammatory bowel disease; (m) multiple myeloma; (n) myasthenia gravis; (O) myocarditis; (p) Paget's disease; (q) periodontal disease; (r) periodontitis; (s) periprosthetic osteolysis; (t) polymyositis; (u) primary biliary cirrhosis; (v) psoriatic arthritis; (w) Sjögren's syndrome; (x) Still's disease; (y) systemic lupus erythematosus; and (z) vasculitis.

In a still further aspect, the present invention provides for a use of the modified VLP, the composition, the pharmaceutical composition or the vaccine composition of the invention for the preparation of a medicament for the therapeutic or prophylactic treatment of autoimmune-diseases and/or of bone-related diseases. Furthermore, in a still further aspect, the present invention provides for a use of a modified VLP, the composition or the pharmaceutical composition of the invention, either in isolation or in combination with other agents, for the manufacture of a composition, vaccine, drug or medicament for therapy or prophylaxis of autoimmune-diseases and/or of bone-related diseases, and/or for stimulating the mammalian immune system.

In a preferred embodiment of the invention, the TNF-peptide of the modified VLP is derived from a vertebrate polypeptide selected from the group consisting of TNFα, LTα and LTα/β, and wherein said autoimmune disease or bone related disease is selected from the group consisting of (a) psoriasis; (b) rheumatoid arthritis; (c) psoriatic arthritis; (d) inflammatory bowel disease; (e) systemic lupus erythematosus; (f) ankylosing spondylitis; (g) Still's disease; (h) polymyositis; (i) vasculitis; (j) diabetes; (k) myasthenia gravis; (l) Sjögren's syndrome; and (m) multiple sclerosis.

In a further preferred embodiment of the invention, the TNF-peptide of the modified VLP is derived from a vertebrate LIGHT polypeptide, and wherein said autoimmune disease or bone related disease is selected from the group consisting of rheumatoid arthritis and diabetes.

In again another preferred embodiment of the invention, the TNF-peptide of the modified VLP is derived from a vertebrate FasL polypeptide, and wherein said autoimmune disease or bone related disease is selected from the group consisting of systemic lupus erythematosus, diabetes, autoimmune thyroid disease, multiple sclerosis and autoimmune hepatitis.

In a further preferred embodiment of the invention, the TNF-peptide of the modified VLP is derived from a vertebrate CD40L polypeptide, and wherein said autoimmune disease or bone related disease is selected from the group consisting of rheumatoid arthritis, atherosclerosis, systemic lupus erythematosus, inflammatory bowel disease and Sjögren's syndrome.

In a further preferred embodiment of the invention, the TNF-peptide of the modified VLP is derived from a vertebrate TRAIL polypeptide, and wherein said autoimmune disease or bone related disease is selected from the group consisting of rheumatoid arthritis, multiple sclerosis and autoimmune thyroid disease.

In a further preferred embodiment of the invention, the TNF-peptide of the modified VLP is derived from a vertebrate RANKL polypeptide, and wherein said autoimmune disease or bone related disease is selected from the group consisting of psoriasis, rheumatoid arthritis, osteoporosis, psoriatic arthritis, periodontis, periodontal disease, periprostetic osteolysis, bone metasis, multiple myeloma, bone cancer pain and Paget's disease.

In a further preferred embodiment of the invention, the TNF-peptide of the modified VLP is derived from a vertebrate CD30L polypeptide, and wherein said autoimmune disease or bone related disease is selected from the group consisting of rheumatoid arthritis, systemic lupus erythematosus, autoimmune thyroid disease, myocarditis, Sjögren's syndrome and primary biliary cirrhosis.

In a further preferred embodiment of the invention, the TNF-peptide of the modified VLP is derived from a vertebrate 4-1BBL polypeptide, and wherein said autoimmune disease or bone related disease is selected from the group consisting of rheumatoid arthritis, inflammatory bowel disease and myocarditis.

In a further preferred embodiment of the invention, the TNF-peptide of the modified VLP is derived from a vertebrate OX40L polypeptide, and wherein said autoimmune disease or bone related disease is selected from the group consisting of rheumatoid arthritis, multiple sclerosis and inflammatory bowel disease.

In a further preferred embodiment of the invention, the TNF-peptide of the modified VLP is derived from a vertebrate BAFF polypeptide, and wherein said autoimmune disease or bone related disease is selected from the group consisting of rheumatoid arthritis, systemic lupus erythematosus and Sjögren's syndrome.

In a further preferred embodiment of the invention, the TNF-peptide of the modified VLP consists of a peptide with a length of 4, 5 or 6 to 18 amino acid residues, preferably with a length of 4, 5 or 6 to 16 amino acid residues, more preferably with a length of 4, 5 or 6 to 14 amino acid residues, and again even more preferably with a length of 6 to 14 amino acid residues.

Therefore, the invention provides, in particular, vaccine compositions which are suitable for preventing and/or reducing or curing autoimmune-diseases and/or of bone-related diseases or conditions related thereto. The invention further provides immunization and vaccination methods, respectively, for preventing and/or reducing or curing autoimmune-diseases and/or of bone-related diseases or conditions related thereto, in animals, and in particular in pets such as cats or dogs, as well as in humans. The inventive compositions may be used prophylactically or therapeutically.

In specific embodiments, the invention provides methods for preventing, curing and/or attenuating autoimmune-diseases and/or of bone-related diseases or conditions related thereto which are caused or exacerbated by “self” gene products, i.e. “self antigens” as used herein. In related embodiments, the invention provides methods for inducing immunological responses in animals and individuals, respectively, which lead to the production of antibodies that prevent, cure and/or attenuate autoimmune-diseases and/or of bone-related diseases or conditions related thereto, which are caused or exacerbated by “self” gene products.

As would be understood by one of ordinary skill in the art, when compositions of the invention are administered to an animal or a human, they may be in a composition which contains salts, buffers, adjuvants, or other substances which are desirable for improving the efficacy of the composition. Examples of materials suitable for use in preparing pharmaceutical compositions are provided in numerous sources including Remington's Pharmaceutical Sciences (Osol, A, ed., Mack Publishing Co. (1990)).

Compositions of the invention are said to be “pharmacologically acceptable” if their administration can be tolerated by a recipient individual. Further, the compositions of the invention will be administered in a “therapeutically effective amount” (i.e., an amount that produces a desired physiological effect).

The compositions of the present invention may be administered by various methods known in the art, but will normally be administered by injection, infusion, inhalation, oral administration or other suitable physical methods. The compositions may alternatively be administered intramuscularly, intravenously, or subcutaneously. Components of compositions for administration include sterile aqueous (e.g., physiological saline) or non-aqueous solutions and suspensions. Examples of non-aqueous solvents are propylene glycol; polyethylene glycol, vegetable oils such as olive oil, and injectable organic esters such as ethyl oleate. Carriers or occlusive dressings can be used to increase skin permeability and enhance antigen absorption.

Other embodiments of the present invention will be apparent to one of ordinary skill in light of what is known in the art, the following description of the invention, and the claims.

### BRIEF DESCRIPTION OF THE FIGURES

FIG. 1: Coupling of mTNFα(4-23) peptide to Qβ capsid protein.

Proteins were analysed on a 12% SDS-polyacrylamide gel under reducing conditions. The gel was stained with Coomassie Brilliant Blue. Molecular weights of marker proteins are given on the left margin, identities of protein bands are indicated on the right margin. Lane 1: Prestained protein marker (New England Biolabs). Lane 2: derivatized Qβ capsid protein. Lane 3: Qβ-TNFα(4-23) peptide coupling reaction (insoluble fraction). Lane 4: Qβ-TNFα(4-23) peptide coupling reaction (soluble fraction).

FIG. 2: Detection of neutralizing antibodies in mice immunized with mTNFα(4-23) peptide coupled to Qβ capsid.

A. Inhibition of mTNFα/mTNFRI interaction. ELISA plates were coated with 10 μg/ml mouse TNFα protein and co-incubated with serial dilutions of mouse sera from day 32 and 0.25 nM mouse TNFRI-hFc fusion protein. Receptor binding was detected with horse raddish peroxidase conjugated anti-hFc antibody.

B. Inhibition of hTNFα/hTNFRI interaction: ELISA plates were coated with 10 μg/1 ml human TNFα protein and co-incubated with serial dilutions of mouse sera from day 32 and 0.25 nM human TNRI-hFc fusion protein. Receptor binding was detected with horse raddish peroxidase conjugated anti-hFc antibody.

FIG. 3: Coupling of mRANKL peptide to Qβ capsid protein.

Proteins were analysed on a 12% SDS-polyacrylamide gel under reducing conditions. The gel was stained with Coomassie Brilliant Blue. Molecular weights of marker proteins are given on the left margin, identities of protein bands are indicated on the right margin. Lane 1: Prestained protein marker (New England Biolabs). Lane 2: derivatized Qβ capsid protein. Lane 3: Qβ-mRANKL(155-174) peptide coupling reaction (insoluble fraction). Lane 4: Qβ-mRANKL(155-174) peptide coupling reaction (soluble fraction).

FIG. 4: Detection of neutralizing antibodies in mice immunized with mRANKL(155-174) peptide coupled to Qβ capsid.

A. Inhibition of mRANKL/mRANK interaction. ELISA plates were coated with 10 μg/ml mouse RANKL protein and co-incubated with serial dilutions of a serum pool of 4 mice which had been immunized with mRANKL(155-174) peptide coupled to Qβ capsid in the absence of Alum (day 35 after first vaccination) and 0.35 nM mouse RANK-hFc fusion protein. Receptor binding was detected with horse raddish peroxidase conjugated anti-hFc antibody.

B. Inhibition of hRANKL/hRANK interaction. ELISA plates were coated with 5 μg/ml human RANKL protein and co-incubated with serial dilutions of a serum pool of 4 mice which had been immunized with mRANKL(155-174) peptide coupled to Qβ capsid in the absence of Alum (day 35 after first vaccination) and 0.35 nM human RANK-hFc fusion protein. Receptor binding was detected with horse raddish peroxidase conjugated anti-hFc antibody.

### DETAILED DESCRIPTION OF THE INVENTION

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the preferred methods and materials are hereinafter described.

1. Definitions

Adjuvant: The term “adjuvant” as used herein refers to non-specific stimulators of the immune response or substances that allow generation of a depot in the host which when combined with the vaccine and pharmaceutical composition, respectively, of the present invention may provide for an even more enhanced immune response. A variety of adjuvants can be used. Examples include complete and incomplete Freund's adjuvant, aluminum hydroxide and modified muramyldipeptide. Further adjuvants are mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and Corynebacterium parvum. Such adjuvants are also well known in the art. Further adjuvants that can be administered with the compositions of the invention include, but are not limited to, Monophosphoryl lipid immunomodulator, AdjuVax 100a, QS-21, QS-18, CRL1005, Aluminum salts (Alum), MF-59, OM-174, OM-197, OM-294, and Virosomal adjuvant technology. The adjuvants can also comprise a mixture of these substances.

Immunologically active saponin fractions having adjuvant activity derived from the bark of the South American tree Quillaja Saponaria Molina are known in the art. For example QS21, also known as QA21, is an Hplc purified fraction from the Quillaja Saponaria Molina tree and it's method of its production is disclosed (as QA21) in U.S. Pat. No. 5,057,540. Quillaja saponin has also been disclosed as an adjuvant by Scott et al., Int. Archs. Allergy Appl. Immun., 1985, 77, 409. Monosphoryl lipid A and derivatives thereof are known in the art. A preferred derivative is 3 de-o-acylated monophosphoryl lipid A, and is known from British Patent No. 2220211. Further preferred adjuvants are described in WO 00/00462, the disclosure of which is herein incorporated by reference.

However, an advantageous feature of the present invention is the high immunogenicity of the modified core particles of the invention, even in the absence of adjuvants. As already outlined herein or will become apparent as this specification proceeds, vaccines and pharmaceutical compositions devoid of adjuvants are provided, in further alternative or preferred embodiments, leading to vaccines and pharmaceutical compositions for treating autoimmune-diseases and/or of bone-related diseases while being devoid of adjuvants and, thus, having a superior safety profile since adjuvants may cause side-effects. The term “devoid” as used herein in the context of vaccines and pharmaceutical compositions for treating autoimmune-diseases and/or of bone-related diseases refers to vaccines and pharmaceutical compositions that are used essentially without adjuvants, preferably without detectable amounts of adjuvants.

Amino acid linker: An “amino acid linker”, or also just termed “linker” within this specification, as used herein, either associates the TNF-peptide of the invention with the second attachment site, or more preferably, already comprises or contains the second attachment site, typically—but not necessarily—as one amino acid residue, preferably as a cysteine residue. The term “amino acid linker” as used herein, however, does not intend to imply that such an amino acid linker consists exclusively of amino acid residues, even if an amino acid linker consisting of amino acid residues is a preferred embodiment of the present invention. The amino acid residues of the amino acid linker are, preferably, composed of naturally occurring amino acids or unnatural amino acids known in the art, all-L or all-D or mixtures thereof. However, an amino acid linker comprising a molecule with a sulfhydryl group or cysteine residue is also encompassed within the invention. Such a molecule comprises preferably a C1-C6 alkyl-, cycloalkyl (C5, C6), aryl or heteroaryl moiety. However, in addition to an amino acid linker, a linker comprising preferably a C1-C6 alkyl-, cycloalkyl-(C5, C6), aryl- or heteroaryl-moiety and devoid of any amino acid(s) shall also be encompassed within the scope of the invention. Association between the TNF-peptide of the invention or optionally the second attachment site and the amino acid linker is preferably by way of at least one covalent bond, more preferably by way of at least one peptide bond.

Animal: As used herein, the term “animal” is meant to include, for example, humans, sheep, elks, deer, mule deer, minks, monkeys, horses, cattle, pigs, goats, dogs, cats, rats, mice, but also birds, chicken, reptiles, fish, insects and arachnids. Preferred animals are vertebrates, more preferred animals are mammals, and even more preferred animals are eutherians.

Antibody: As used herein, the term “antibody” refers to molecules which are capable of binding an epitope or antigenic determinant. The term is meant to include whole antibodies and antigen-binding fragments thereof, including single-chain antibodies. Most preferably the antibodies are human antigen binding antibody fragments and include, but are not limited to, Fab, Fab′ and F(ab′)2, Fd, single-chain Fvs (scFv), single-chain antibodies, disulfide-linked Fvs (sdFv) and fragments comprising either a V_{L} or V_{H} domain. The antibodies can be from any animal origin including birds and mammals. Preferably, the antibodies are human, murine, rabbit, goat, rat, guinea pig, camel, horse or chicken. As used herein, “human” antibodies include antibodies having the amino acid sequence of a human immunoglobulin and include antibodies isolated from human immunoglobulin libraries or from animals transgenic for one or more human immunoglobulins and that do not express endogenous immunoglobulins, as described, for example, in U.S. Pat. No. 5,939,598 by Kucherlapati et al.

Antigen: As used herein, the term “antigen” refers to a molecule capable of being bound by an antibody or a T-cell receptor (TCR) if presented by MHC molecules. The term “antigen”, as used herein, also encompasses T-cell epitopes. An antigen is additionally capable of being recognized by the immune system and/or being capable of inducing a humoral immune response and/or cellular immune response leading to the activation of B- and/or T-lymphocytes. This may, however, require that, at least in certain cases, the antigen contains or is linked to a Th cell epitope and is given in adjuvant. An antigen can have one or more epitopes (B- and T-cell epitopes). The specific reaction referred to above is meant to indicate that the antigen will preferably react, typically in a highly selective manner, with its corresponding antibody or TCR and not with the multitude of other antibodies or TCRs which may be evoked by other antigens. Antigens as used herein may also be mixtures of several individual antigens. Preferred antigens, and thus preferred TNF-peptides, are short peptides (4-8 aa residues, preferably 6-8 aa residues) which do not result in a T-cell response (B-cell epitopes only).

Antigenic determinant: As used herein, the term “antigenic determinant” is meant to refer to that portion of an antigen that is specifically recognized by either B- or T-lymphocytes. B-lymphocytes responding to antigenic determinants produce antibodies, whereas T-lymphocytes respond to antigenic determinants by proliferation and establishment of effector functions critical for the mediation of cellular and/or humoral immunity.

Association: As used herein, the term “association” as it applies to the first and second attachment sites, refers to the binding of the first and second attachment sites that is preferably by way of at least one non-peptide bond. The nature of the association may be covalent, ionic, hydrophobic, polar, or any combination thereof, preferably the nature of the association is covalent.

Attachment Site, First: As used herein, the phrase “first attachment site” refers to an element of non-natural or natural origin, to which the second attachment site located on the TNF-peptide of the invention may associate. The first attachment site may be a protein, a polypeptide, an amino acid, a peptide, a sugar, a polynucleotide, a natural or synthetic polymer, a secondary metabolite or compound (biotin, fluorescein, retinol, digoxigenin, metal ions, phenylmethylsulfonylfluoride), or a chemically reactive group such as an amino group, a carboxyl group, a sulfhydryl group, a hydroxyl group, a guanidinyl group, histidinyl group, or a combination thereof. The first attachment site is located, typically and preferably on the surface, of the core particle such as, preferably the virus-like particle. Multiple first attachment sites are present on the surface of the core and virus-like particle, respectively, typically in a repetitive configuration. In a preferred embodiment the first attachment site is associated with the VLP, through at least one covalent bond, preferably through at least one peptide bond. In a further preferred embodiment the first attachment site is naturally occurring with the VLP. Alternatively, in a preferred embodiment the first attachment site is artificially added to the VLP.

Attachment Site, Second: As used herein, the phrase “second attachment site” refers to an element associated with the TNF-peptide of the invention to which the first attachment site located on the surface of the core particle and virus-like particle, respectively, may associate. The second attachment site of the TNF-peptide may be a protein, a polypeptide, a peptide, a sugar, a polynucleotide, a natural or synthetic polymer, a secondary metabolite or compound (biotin, fluorescein, retinol, digoxigenin, metal ions, phenylmethylsulfonylfluoride), or a chemically reactive group such as an amino group, a carboxyl group, a sulflhydryl group, a hydroxyl group, a guanidinyl group, histidinyl group, or a combination thereof. In certain embodiments of the invention at least one second attachment site may be added to the TNF-peptide of the invention. The term “TNF-peptide of the invention with at least one second attachment site” refers, therefore, to a TNF-peptide of the invention comprising at least the TNF-peptide of the invention and a second attachment site. However, in particular for a second attachment site, which is of non-natural origin, i.e. not naturally occurring within the TNF-peptide of the invention, these modified TNF-peptides of the invention can also comprise an “amino acid linker”.

Bound: As used herein, the term “bound” as well as the term “linked”, which is herein used equivalently, refers to binding or attachment that may be covalent, e.g., by chemically coupling, or non-covalent, e.g., ionic interactions, hydrophobic interactions, hydrogen bonds, etc. Covalent bonds can be, for example, ester, ether, phosphoester, amide, peptide, imide, carbon-sulfur bonds such as thioether, carbon-phosphorus bonds, and the like. In certain preferred embodiments the first attachment site and the second attachment site are linked through (i) at least one covalent bond, or (ii) at least one non-peptide bond, preferably through at least one covalent non-peptide bond, and even more preferably through exclusively non-peptide bonds, and hereby further preferably through exclusively non-peptide and covalent bonds. The term “linked” as used herein, however, shall not only encompass a direct linkage of the at least one TNF-peptide and the virus-like particle but also, alternatively and preferably, an indirect linkage of the at least one TNF-peptide and the virus-like particle through intermediate molecule(s), and hereby typically and preferably by using at least one, preferably one, heterobifunctional cross-linker. Moreover, the term “linked” as used herein shall not only encompass a direct linkage of the at least one first attachment site and the at least one second attachment site but also, alternatively and preferably, an indirect linkage of the at least one first attachment site and the at least one second attachment site through intermediate molecule(s), and hereby typically and preferably by using at least one, preferably one, heterobifunctional cross-linker.

Coat protein(s): As used herein, the term “coat protein(s)” refers to the protein(s) of a bacteriophage or a RNA-phage capable of being incorporated within the capsid assembly of the bacteriophage or the RNA-phage. However, when referring to the specific gene product of the coat protein gene of RNA-phages the term “CP” is used. For example, the specific gene product of the coat protein gene of RNA-phage Qβ is referred to as “Qβ CP”, whereas the “coat proteins” of bacteriophage Qβ comprise the “Qβ CP” as well as the A1 protein. The capsid of Bacteriophage Qβ is composed mainly of the Qβ CP, with a minor content of the A1 protein. Likewise, the VLP Qβ coat protein contains mainly Qβ CP, with a minor content of A1 protein.

Core particle: As used herein, the term “core particle” refers to a rigid structure with an inherent repetitive organization. A core particle as used herein may be the product of a synthetic process or the product of a biological process.

Effective Amount: As used herein, the term “effective amount” refers to an amount necessary or sufficient to realize a desired biologic effect. An effective amount of the composition would be the amount that achieves this selected result, and such an amount could be determined as a matter of routine by a person skilled in the art. For example, an effective amount for treating an immune system deficiency could be that amount necessary to cause activation of the immune system, resulting in the development of an antigen specific immune response upon exposure to antigen. The term is also synonymous with “sufficient amount.”

The effective amount for any particular application can vary depending on such factors as the disease or condition being treated, the particular composition being administered, the size of the subject, and/or the severity of the disease or condition. One of ordinary skill in the art can empirically determine the effective amount of a particular composition of the present invention without necessitating undue experimentation.

Epitope: As used herein, the term “epitope” refers to continuous or discontinuous portions of a polypeptide having antigenic or immunogenic activity in an animal, preferably a mammal, and most preferably in a human. An epitope is recognized by an antibody or a T cell through its T cell receptor in the context of an MHC molecule. An “immunogenic epitope,” as used herein, is defined as a portion of a polypeptide that elicits an antibody response or induces a T-cell response in an animal, as determined by any method known in the art. (See, for example, Geysen et al., Proc. Natl. Acad. Sci. USA 81:3998-4002 (1983)). The term “antigenic epitope,” as used herein, is defined as a portion of a protein to which an antibody can immunospecifically bind its antigen as determined by any method well known in the art. Immunospecific binding excludes non-specific binding but does not necessarily exclude cross-reactivity with other antigens. Antigenic epitopes need not necessarily be immunogenic. Antigenic epitopes can also be T-cell epitopes, in which case they can be bound immunospecifically by a T-cell receptor within the context of an MHC molecule.

An epitope can comprise 3 amino acids in a spatial conformation which is unique to the epitope. Generally, an epitope consists of at least about 4 of such amino acids, and more usually, consists of at least about 4, 5, 6, 7, 8, 9, or 10 of such amino acids. If the epitope is an organic molecule, it may be as small as Nitrophenyl. Preferred epitopes are the TNF-peptides of the invention, which are believed to be B-type epitopes.

Fusion: As used herein, the term “fusion” refers to the combination of amino acid sequences of different origin in one polypeptide chain by in-frame combination of their coding nucleotide sequences. The term “fusion” explicitly encompasses internal fusions, i.e., insertion of sequences of different origin within a polypeptide chain, in addition to fusion to one of its termini.

TNF-superfamily member: The term “TNF-superfamily member” as used herein refers to a protein comprising a TNF-like domain. As used herein “TNF-superfamily member” includes all forms of TNF-superfamily members known in humans, cats, dog, mice, rats, eutherians in general, mammals in general as well as of other animals. The structure of the founding member TNF has been determined to a resolution of 2.9 Angstrom using X-ray crystallography. The protein is trimeric, each subunit consisting of an anti-parallel beta-sandwich. The subunits trimerise via a novel edge-to-face packing of beta-sheets. Comparison of the subunit fold with that of other proteins reveals similarity to the ‘jelly-roll’ structural motif characteristic of viral coat proteins. TNF-superfamily members comprise a globular TNF-like extracellular domain of about 150 residues, which domain is classified as cd00184, pfam00229 or smart00207 in the conserved domain database CDD (Marchler-Bauer A, et al. (2003), “CDD: a curated Entrez database of conserved domain alignments”, Nucleic Acids Res. 31: 383-387). Furthermore, proteins of the TNF-superfamily generally have an intracellular N-terminal domain, a short transmembrane segment, an extracellular stalk, and said globular TNF-like extracellular domain of about 150 residues. Some members differ somewhat from this general configuration (see below). It is believed that generally each TNF molecule has three receptor-interaction sites (between the three subunits), thus allowing signal transmission by receptor clustering. TNF-alpha is synthesized as a type II membrane protein which then undergoes post-translational cleavage liberating the extracellular domain. CD27L, CD30L, CD40L, FASL, LT-beta, 4-1BBL and TRAIL also appear to be type II membrane proteins. LT-alpha is a secreted protein. All these cytokines seem to form homotrimeric (or heterotrimeric in the case of LT-alpha/beta) complexes that are recognized by their specific receptors.

Some family members can initiate apoptosis by binding to related receptors, some of which have intracellular death domains. TNF superfamily members as used herein include: TNFα, LTα, LTα/β, FasL, CD40L, TRAIL, RANKL, CD30L, 4-1BBL, OX40L, GITRL and BAFF, CD27L, TWEAK, APRIL, TL1A, EDA and any other polypeptide, in which a TNF-like domain can be identified. Such identification can be effected by various ways known to those skilled in the art, for example, by the programm BlastP (protein-protein Blast) accessible on, for example, the webpage of the NCBI under the URL http://www.ncbi.nlm.nih.gov/BLAST/. Domain identification can be carried out by using the default settings of the Blastp programm: choose database=nr, Do CD-search=on, Options for advanced blasting: select from =all organisms, composition-based statistics=on, choose filter=low complexity, expect=10, word size=3, Matrix=Blosum 62, gap costs=existence 11 extension 1. Such a search will help to detect a TNF-like domain in a queried polypeptide having a TNF-like domain.

TNF-superfamily members, as used herein, include TNF-superfamily members with or without protein modification, such as phosphorylation, glycosylation or ubiquitination. Moreover, the term TNF-superfamily member also includes all splice variants that exist of a TNF-superfamily member. In addition, due to high sequence homology between the same TNF-superfamily member of different species, all natural variants and variants generated by genetic engineering of TNF-superfamily members with more than 80% identity, preferably more than 90%, more preferably more than 95%, and even more preferably more than 99% with the respective human TNF-superfamily member are referred to as “TNF-superfamily member” herein.

As used herein, the term “TNF-peptide” or “TNF peptide of the invention” is a peptide comprising a peptide sequence homologous to, that is in this context corresponding to, amino acid residues 3 to 8 of the consensus sequence for the conserved domain pfam 00229 (SEQ ID NO:1), preferably a peptide sequence homologous to amino acid residues 1 to 8 of the consensus sequence for the conserved domain pfam 00229 (SEQ ID NO: 1), even more preferred a peptide sequence homologous to amino acid residues 1-13 fo said consensus sequence. When the TNF-peptide is a peptide from human or mouse TNFα, said TNF-peptide consists of a peptide with a length of 4, 5 or 6 to 18 amino acid residues, preferably with a length of 4, 5 or 6 to 16 amino acid residues, more preferably with a length of 4, 5 or 6 to 14 amino acid residues; and when the TNF-peptide is a peptide from human or mouse RANKL, from human or mouse LTα, or from human or mouse LTβ, or from human or mouse LTα/LTβ said TNF-peptide consists of a peptide with a length of 4, 5 or 6 to 50 amino acid residues, preferably with a length of 4, 5 or 6 to 40 amino acid residues, more preferably with a length of 4, 5 or 6 to 30 amino acid residues. A homologous peptide is such a peptide which is derived from a TNF-superfamily member of an animal, including a human being, particularly a mammalian TNF superfamily member, like e.g. mouse or human RANKL or mouse or human TNFα, and represents those amino acid residues that correspond to SEQ ID NO:1. These homologous peptides are identifiable to a skilled person by way of aligning the consensus sequence of the TNF superfamily (SEQ ID NO:1) with said TNF-superfamily member of the other animal. As explained above, a TNF-peptide comprises a peptide sequence corresponding to the above-mentioned amino acid residues of the consensus sequence. That is, outside of the specified homology region with the consensus sequence (e.g. amino acid residues 3 to 8 of the consensus sequence) the TNF-peptide may differ from a polypeptide that is a TNF-superfamily member. Preferably, however, that part of a TNF-peptide that is outside of the above-specified homology region with the consensus sequence, is at least 70% identical, more preferably at least 75%, 80%, 85%, 90%, 95%, 99% or even 100% identical with a polypeptide that is a TNF-superfamily member. Preferred are mammalian TNF-superfamily members, more preferred are human TNF-superfamily members.

In such cases, where the TNF-peptides of the invention are comprised within a larger context, i.e. a fusion polypeptide or a TNF-peptide with an added linker peptide or attachment site, the TNF-peptide of the invention is preferably not followed by that amino acid residue which follows it in the context of the polypeptide from which the TNF-peptide is derived.

The TNF-peptide may be obtained by recombinant expression in eukaryotic or prokaryotic expression systems as TNF-peptide alone, but preferably as a fusion with other amino acids or proteins, e.g. to facilitate folding, expression or solubility of the TNF-peptide or to facilitate purification of the TNF-peptide. Preferred are fusions between TNF-peptides and subunit proteins of VLPs or capsids. In such a case, one or more amino acids may be added N- or C-terminally to TNF-peptides, but it is preferred that the TNF-peptide is at the N-terminus of a fusion polypeptide, i.e. coupled or linked via its own C-terminus to its fusion partner.

Alternatively and preferably, to enable coupling of TNF-peptides to subunit proteins of VLPs or capsids or core particles, at least one second attachment site may be added to the TNF-peptide. Alternatively TNF-peptides may be synthesized using methods known to the art, in particular by organic-chemical peptide synthesis. Such peptides may even contain amino acids which are not present in the corresponding TNF superfamily member protein. The peptides may be modified by, e.g., phosphorylation, but this modification is not necessary for effective modified VLPs of the invention.

Residue: As used herein, the term “residue” is meant to mean a specific amino acid in a polypeptide backbone or side chain.

Immune response: As used herein, the term “immune response” refers to a humoral immune response and/or cellular immune response leading to the activation or proliferation of B- and/or T-lymphocytes and/or and antigen presenting cells.

In some instances, however, the immune responses may be of low intensity and become detectable only when using at least one substance in accordance with the invention. “Immunogenic” refers to an agent used to stimulate the immune system of a living organism, so that one or more functions of the immune system are increased and directed towards the immunogenic agent. A substance which “enhances” an immune response refers to a substance in which an immune response is observed that is greater or intensified or deviated in any way with the addition of the substance when compared to the same immune response measured without the addition of the substance.

Immunization: As used herein, the terms “immunize” or “immunization” or related terms refer to conferring the ability to mount a substantial immune response (comprising antibodies and/or cellular immunity such as effector CTL) against a target antigen or epitope. These terms do not require that complete immunity be created, but rather that an immune response be produced which is substantially greater than baseline. For example, a mammal may be considered to be immunized against a target antigen if the cellular and/or humoral immune response to the target antigen occurs following the application of methods of the invention.

Natural origin: As used herein, the term “natural origin” means that the whole or parts thereof are not synthetic and exist or are produced in nature.

Non-natural: As used herein, the term generally means not from nature, more specifically, the term means from the hand of man.

Non-natural origin: As used herein, the term “non-natural origin” generally means synthetic or not from nature; more specifically, the term means from the hand of man.

Ordered and repetitive antigen or antigenic determinant array: As used herein, the term “ordered and repetitive antigen or antigenic determinant array” generally refers to a repeating pattern of antigen or antigenic determinant, characterized by a typically and preferably uniform special arrangement of the antigens or antigenic determinants with respect to the core particle and virus-like particle, respectively. In one embodiment of the invention, the repeating pattern may be a geometric pattern. Typical and preferred examples of suitable ordered and repetitive antigen or antigenic determinant arrays are those which possess strictly repetitive paracrystalline orders of antigens or antigenic determinants, preferably with spacings of 1 to 30 nanometers, preferably 2 to 15 nanometers, even more preferably 2 to 10 nanometers, even again more preferably 2 to 8 nanometers, and further more preferably 3 to 7 nanometers.

Pili: As used herein, the term “pili” (singular being “pilus”) refers to extracellular structures of bacterial cells composed of protein monomers (e.g., pilin monomers) which are organized into ordered and repetitive patterns. Further, pili are structures which are involved in processes such as the attachment of bacterial cells to host cell surface receptors, inter-cellular genetic exchanges, and cell-cell recognition. Examples of pili include Type-1 pili, P-pili, F1C pili, S-pili, and 987P-pili. Additional examples of pili are set out below.

Pilus-like structure: As used herein, the phrase “pilus-like structure” refers to structures having characteristics similar to that of pili and composed of protein monomers. One example of a “pilus-like structure” is a structure formed by a bacterial cell which expresses modified pilin proteins that do not form ordered and repetitive arrays that are identical to those of natural pili.

Polypeptide: As used herein, the terms “polypeptide” and “peptide” refer to molecules composed of monomers (amino acids) linearly linked by amide bonds (also known as peptide bonds). They indicate a molecular chain of amino acids. Preferred peptides of the invention are pentapeptides, hexapeptides, heptapeptides, octapeptides nonapeptides, decapeptides and all other peptides with a length of up to and including 300, preferably 250, even more preferably 200, again more preferably 150, and further more preferably 100, and again further preferably 75, and again more preferably 50 amino acid residues. A polypeptide is composed of more than 300 amino acid residues and up to 10000, for the purposes of this invention. For the purpose of this invention, a protein is regarded as a polypeptide. These terms also refer to post-expression modifications of the polypeptide or peptide, for example, glycosylations, acetylations, phosphorylations, and the like. A recombinant or derived polypeptide or peptide is not necessarily translated from a designated nucleic acid sequence. It may also be generated in any manner, including chemical synthesis, which is preferred for peptides.

Self antigen: As used herein, the term “self antigen” refers to proteins encoded by the host's DNA and products generated by proteins or RNA encoded by the host's DNA are defined as self. In addition, proteins that result from a combination of two or several self-molecules may also be considered self.

Treatment: As used herein, the terms “treatment”, “treat”, “treated” or “treating” refer to prophylaxis and/or therapy. When used with respect to an autoimmune or bone related (AI or BR) disease, for example, the term refers to a prophylactic treatment which increases the resistance of a subject to develop an AI or BR disease or, in other words, decreases the likelihood that the subject will develop an AI or BR or will show signs of illness attributable to an AI or an BR, as well as a treatment after the subject has developed an AI or BR in order to fight the AI or BR, e.g., reduce or eliminate the AI or BR or prevent it from becoming worse.

Vaccine: As used herein, the term “vaccine” refers to a formulation which contains the modified core particle, and in particular the modified VLP of the present invention and which is in a form that is capable of being administered to an animal. Typically, the vaccine comprises a conventional saline or buffered aqueous solution medium in which the composition of the present invention is suspended or dissolved. In this form, the composition of the present invention can be used conveniently to prevent, ameliorate, or otherwise treat a condition. Upon introduction into a host, the vaccine is able to provoke an immune response including, but not limited to, the production of antibodies and/or cytokines and/or the activation of cytotoxic T cells, antigen presenting cells, helper T cells, dendritic cells and/or other cellular responses. Typically, the modified core particle of the invention, and preferably, the modified VLP of the invention, preferably induces a predominant B-type response, more preferably a B-type response only, which can be a further advantage.

Optionally, the vaccine of the present invention additionally includes an adjuvant which can be present in either a minor or major proportion relative to the compound of the present invention.

Virus-like particle (VLP): As used herein, the term “virus-like particle” refers to a structure resembling a virus particle. Moreover, a virus-like particle in accordance with the invention is non-replicative and noninfectious since it lacks all or part of the viral genome, in particular the replicative and infectious components of the viral genome. A virus-like particle in accordance with the invention may contain nucleic acid distinct from their genome. A typical and preferred embodiment of a virus-like particle in accordance with the present invention is a viral capsid such as the viral capsid of the corresponding virus, bacteriophage, or RNA-phage. The terms “viral capsid” or “capsid”, as interchangeably used herein, refer to a macromolecular assembly composed of viral protein subunits. Typically and preferably, the viral protein subunits assemble into a viral capsid and capsid, respectively, having a structure with an inherent repetitive organization, wherein said structure is, typically, spherical or tubular. For example, the capsids of RNA-phages or HBcAgs have a spherical form of icosahedral symmetry. The term “capsid-like structure” as used herein, refers to a macromolecular assembly composed of viral protein subunits resembling the capsid morphology in the above defined sense but deviating from the typical symmetrical assembly while maintaining a sufficient degree of order and repetitiveness.

Virus-like particle of a bacteriophage: As used herein, the term “virus-like particle of a bacteriophage” or the term “virus-like particle of a RNA-phage” which is herein used equivalently, refers to a virus-like particle resembling the structure of a bacteriophage, being non replicative and/or non-infectious, and lacking at least the gene or genes encoding for the replication machinery of the bacteriophage, and typically also lacking the gene or genes encoding the protein or proteins responsible for viral attachment to or entry into the host. This definition should, however, also encompass virus-like particles of bacteriophages, in which the aforementioned gene or genes are still present but inactive, and, therefore, also leading to non-replicative and noninfectious virus-like particles of a bacteriophage.

VLP of RNA phage coat protein: The capsid structure formed from the self-assembly of 180 subunits of RNA phage coat protein and optionally containing host RNA is referred to as a “VLP of RNA phage coat protein.” A specific example is the VLP of Qβ coat protein. In this particular case, the VLP of Qβ coat protein may either be assembled exclusively from Qβ CP subunits (generated by expression of a Qβ CP gene containing, for example, a TAA stop codon precluding any expression of the longer A1 protein through suppression, see Kozlovska, T. M., et al., Intervirology 39: 9-15 (1996)), or additionally contain A1 protein subunits in the capsid assembly.

Virus particle: The term “virus particle” as used herein refers to the morphological form of a virus. In some virus types it comprises a genome surrounded by a protein capsid; others have additional structures (e.g., envelopes, tails, etc.).

One, a, or an: When the terms “one,” “a,” or “an” are used in this disclosure, they mean “at least one” or “one or more,” unless otherwise indicated. Preferably, they mean “one”.

As will be clear to those skilled in the art, certain embodiments of the invention involve the use of recombinant nucleic acid technologies such as cloning, polymerase chain reaction, the purification of DNA and RNA, the expression of recombinant proteins in prokaryotic and eukaryotic cells, etc. Such methodologies are well known to those skilled in the art and can be conveniently found in published laboratory methods manuals (e.g., Sambrook, J. et al., eds., Molecular Cloning, A Laboratory Manual, 2^{nd} edition, Cold Spring Harbor Laboratory Press, Cold Spring Harbor, N.Y. (1989); Ausubel, F. et al., eds., Current Protocols in Molecular Biology, John H. Wiley & Sons, Inc. (1997)). Fundamental laboratory techniques for working with tissue culture cell lines (Celis, J., ed., Cell Biology, Academic Press, 2^{nd} edition, (1998)) and antibody-based technologies (Harlow, E. and Lane, D., Antibodies: A Laboratory Manual, Cold Spring Harbor Laboratory, Cold Spring Harbor, N.Y. (1988); Deutscher, M. P., “Guide to Protein Purification,” Meth. Enzymol. 128, Academic Press San Diego (1990); Scopes, R. K., Protein Purification Principles and Practice, 3^{rd} ed., Springer-Verlag, New York (1994)) are also adequately described in the literature, all of which are incorporated herein by reference.

2. Compositions and Methods for Enhancing an Immune Response

The disclosed invention provides compositions and methods for enhancing an immune response against a TNF-peptide in an animal, preferably a human being. Compositions of the invention comprise, or alternatively consist of (a) a core particle, and preferably a VLP; and (b) at least one peptide (TNF-peptide) comprising a peptide sequence homologous to amino acid residues 3 to 8 of the consensus sequence for the conserved domain pfam 00229 (SEQ ID NO: 1), preferably a peptide sequence homologous to amino acid residues 1 to 8 of the consensus sequence for the conserved domain pfam 00229 (SEQ ID NO:1), wherein a) and b) are linked with one another. Said TNF-peptide consists of a peptide with a length of 4, 5 or 6 to 18 amino acid residues, preferably with a length of 4, 5 or 6 to 16 amino acid residues, more preferably with a length of 4, 5 or 6 to 14 amino acid residues, when the TNF-peptide is a peptide from human or mouse TNFα. Preferred TNF-peptides from TNFα comprise, and more preferably consist of, the peptide VAHVVA (SEQ ID NO:31), more preferably they comprise, or even consist of, the peptide KPVAHVVA (SEQ ID NO:32), even more preferred they comprise, or even consist of, the peptide KPVAHVVAN (SEQ ID NO:33) or SKPVAHVVAN (SEQ ID NO:127), most preferably KPVAHVVAN (SEQ ID NO:33). In a further preferred embodiment the TNF-peptides from TNFα comprise, and more preferably consist of, the peptide SDKPVAHVVANHQ (SEQ ID NO:153). In a preferred embodiment, the TNF-peptide with the second attachment site comprises, and more preferably consists of, the peptide CGGKPVAHVVA (SEQ ID NO:2) or CGGSKPVAHVVAN (SEQ ID NO:146) or CGGSDKPVAHVVANHQ (SEQ ID NO:3).

In a preferred embodiment the TNF-peptide of the invention is bound to the virus-like particle so as to form an ordered and repetitive antigen-VLP-array. In a further preferred embodiment the TNF-peptide consisting of a peptide with a length of 4, 5 or 6 to 75 amino acid residues, preferably with a length of from 4, 5 or 6 to 50 amino acid residues, more preferably with a length of from 4, 5 or 6 to 40 amino acid residues, more preferably with a length of from 4, 5 or 6 to 40 amino acid residues, again more preferably with a length of from 4, 5 or 6 to 30 amino acid residues, even more preferably with a length of from 4, 5 or 6 to 25 amino acid residues, even more preferably with a length of from 4, 5 or 6 to 20 amino acid residues, even more preferably with a length of from 4, 5 or 6 to 18 amino acid residues, even more preferred with a length of from 4, 5 or 6 to 16 amino acid residues, even more preferably with a length of from 4, 5 or 6 to 14 amino acid residues, even more preferably with a length of from 4, 5 or 6 to 13 amino acid residues, even more preferably with a length of from 4, 5 or 6 to 12 amino acid residues. Alternatively, the lower limit in the above-mentioned length ranges (4 to 50, 4 to 40, 4 to 30, 4 to 25, 4 to 20, 4 to 18, 4 to 16, 4 to 14, 4 to 13 and 4 to 12) can preferably be 5, 6, 7 or 8 amino acid residues.

In a further preferred embodiment the TNF-peptide is derived from a vertebrate, preferably a mammalian, more preferably a eutherian polypeptide selected from the group consisting of TNFα, LTα, LTα/β, FasL, CD40L, TRAIL, RANKL, CD30L, 4-1BBL, OX40L, GITRL and BAFF, CD27L, TWEAK, APRIL, TL1A, EDA, preferably selected from the group consisting of TNFα, LTα and LTα/β, or selected from the group consisting of TRAIL and RANKL, or selected from the group consisting of FasL, CD40L, CD30L and BAFF, or selected from the group consisting of 4-1BBL, OX40L and LIGHT, or selected from the group consisting of LTα, LTα/β, FasL, CD40L, TRAIL, CD30L, 4-1BBL, OX40L, GITRL and BAFF.

When the TNF-peptide is derived from LTα, said TNF-peptide preferably comprises, or even consists of, the peptide AAHLVG (SEQ ID NO:34) or the peptide AAHLIG (SEQ ID NO:35), more preferably said TNF-peptide comprises, or even consists of, the peptide KPAAHLVG (SEQ ID NO:36) or KPAAHLIG (SEQ ID NO:37), even more preferably it comprises, or even consists of, the peptide LKPAAHLVG (SEQ ID NO:38) or LKPAAHLIG (SEQ ID NO:39) or HLAHSTLKPAAHLIGDPSKQ (SEQ ID NO:132).

When the TNF-peptide is derived from LTβ, said TNF-peptide preferably comprises, or even consists of, the peptide AAHLIG (SEQ ID NO:40), more preferably it comprises, or even consists of, the peptide PAAHLIGA (SEQ ID NO:41) or the peptide PAAHLIGI (SEQ ID NO:42) or ETDLNPELPAAHLIGAWMSG (SEQ ID NO:130) or ETDLSPGLPAAHLIGAPLKG (SEQ ID NO:131). In a preferred embodiment, the TNF-peptide with the second attachment site comprises, and more preferably consists of, the peptide CGGETDLNPELPAAHLIGAWMSG (SEQ ID NO:152),

When the TNF-peptide is derived from CD40L, said TNF-peptide preferably comprises, or even consists of, the peptide AAHVIS (SEQ ID NO:43) or the peptide AAHVVS (SEQ ID NO:44), more preferably said TNF-peptide comprises, or even consists of, the peptide QIAAHVIS (SEQ ID NO:45) or RIAAHVIS (SEQ ID NO:46), even more preferably it comprises, or even consists of, the peptide NPQIAAHVIS (SEQ ID NO:47) or DPQIAAHVIS (SEQ ID NO:48) or DPQIAAHVVS (SEQ ID NO:49) or EPQIAAHVIS (SEQ ID NO: 50) or QRGDEDPQIAAHVVSEANSN (SEQ ID NO:128) or QKGDQNPQIAAHVISEASSK (SEQ ID NO:133). In a preferred embodiment, the TNF-peptide with the second attachment site comprises, and more preferably consists of, the peptide CGGQRGDEDPQIAAHVVSEANSN (SEQ ID NO:150).

When the TNF-peptide is derived from FasL, said TNF-peptide preferably comprises, or even consists of, the peptide VAHLTG (SEQ ID NO:51), more preferably said TNF-peptide comprises, or even consists of, the peptide RSVAHLTG (SEQ ID NO:52) or RKVAHLTG (SEQ ID NO:53) or RRAAHLTG (SEQ ID NO:54) or KKAAHLTG (SEQ ID NO:55) or PPEKKELRKVAHLTGKSNSR (SEQ ID NO:134).

When the TNF-peptide is derived from CD27L, said TNF-peptide preferably comprises, or even consists of, the peptide AELQLN (SEQ ID NO:56) or LQLNLT (SEQ ID NO:57) or LQLNHT (SEQ ID NO:58), more preferably said TNF-peptide comprises, or even consists of, the peptide VAELQLN (SEQ ID NO:59) or TAELQLN (SEQ ID NO60), even more preferably it comprises, or even consists of, the peptide TAELQLNL (SEQ ID NO:61) or VAELQLNL (SEQ ID NO:62) or VAELQLNH (SEQ ID NO:63) or LGWDVAELQLNHTGPQQDPR (SEQ ID NO:135).

When the TNF-peptide is derived from TRAIL, said TNF-peptide preferably comprises, or even consists of, the peptide AAHIT (SEQ ID NO:64) or the peptide AAHLT (SEQ ID NO:65), more preferably said TNF-peptide comprises, or even consists of, the peptide VAAHITG (SEQ ID NO:66), even more preferably it comprises, or even consists of, the peptide PQKVAAHITG (SEQ ID NO:67) or PQRVAAHITG (SEQ ID NO:68) or ERGPQRVAAHITGTRGRS (SEQ ID NO:136).

When the TNF-peptide is derived from RANKL, said TNF-peptide preferably comprises, or even consists of, the peptide FAHLTI (SEQ ID NO:69) or the peptide SAHLTV (SEQ ID NO:70), more preferably said TNF-peptide comprises, or even consists of, the peptide EAQPFAHLTI (SEQ ID NO:71) or QPFAHLTIN (SEQ ID NO:72), even more preferably it comprises, or even consists of, the peptide KPEAQPFAHLTINA (SEQ ID NO:73) or KLEAQPFAHLTINA (SEQ ID NO:74) or KRSKLEAQPFAHLTINATDI (SEQ ID NO:75) or QRGKPEAQPFAHLTINAASI (SEQ ID NO:76) or EAQPFAHLTINA (SEQ ID NO:149) or AQPFAHLTIN (SEQ ID NO:125). In a preferred embodiment, the TNF-peptide with the second attachment site comprises, and more preferably consists of, the peptides CGGKRSKLEAQPFAHLTINATDI (SEQ ID NO:148) or CGGQRGKPEAQPFAHLTINAASI (SEQ ID NO:30) or CGGQPFAHLTIN (SEQ ID NO:22) or CGGAQPFAHLTIN (SEQ ID NO:147) or CGGEAQPFAHLTINA (SEQ ID NO:23).

When the TNF-peptide is derived from TWEAK, said TNF-peptide preferably comprises, or even consists of, the peptide AAHYEV (SEQ ID NO:77), more preferably said TNF-peptide comprises, or even consists of, the peptide RAIAAHYEV (SEQ ID NO:78) or AAHYEVHP (SEQ ID NO:79), even more preferably it comprises, or even consists of, the peptide ARRAIAAHYEVHP (SEQ ID NO:80) or PRRAIAAHYEVHP (SEQ ID NO:81) or RKTRARRAIAAHYEVHPRPG (SEQ ID NO:).

When the TNF-peptide is derived from APRIL, said TNF-peptide preferably comprises, or even consists of, the peptide SVLHLV (SEQ ID NO:82), more preferably said TNF-peptide comprises, or even consists of, the peptide HSVLHLVP (SEQ ID NO:83) or QSVLHLVP (SEQ ID NO:84), even more preferably it comprises, or even consists of, the peptide KKQHSVLHLVP (SEQ ID NO:85) or KKKHSVLHLVP (SEQ ID NO:86) or KKKQSVLHLVP (SEQ ID NO:87) QKQKKQHSVLHLVPINATS (SEQ ID NO:137).

When the TNF-peptide is derived from BAFF, said TNF-peptide preferably comprises, or even consists of, the peptide LQLIAD (SEQ ID NO:88), more preferably said TNF-peptide comprises, or even consists of, the peptide QDCLQLIADS (SEQ ID NO:89) or QACLQLIADS (SEQ ID NO:90) or NLRNIIQDSLQLIADSDTPT (SEQ ID NO:129) or VTQDCLQLIADSETPT (SEQ ID NO:138). In a preferred embodiment, the TNF-peptide with the second attachment site comprises, and more preferably consists of, the peptide CGGNLRNIIQDSLQLIADSDTPT (SEQ ID NO:151),

When the TNF-peptide is derived from LIGHT, said TNF-peptide preferably comprises, or even consists of, the peptide AAHLTG (SEQ ID NO:91), more preferably said TNF-peptide comprises, or even consists of, the peptide NPAAHLTG (SEQ ID NO 92) or AAHLTGAN (SEQ ID NO:93), even more preferably it comprises, or even consists of, the peptide VNPAAHLTGANS (SEQ ID NO:94) or ANPAAHLTGANA (SEQ ID NO:95) ERRSHEVNPAAHLTGANSSL (SEQ ID NO:139).

When the TNF-peptide is derived from TL1A, said TNF-peptide preferably comprises, or even consists of, the peptide RAHLTV (SEQ ID NO:96) or the peptide RAHLTI (SEQ ID NO:97) or the peptide KAHLTI (SEQ ID NO:98) or the peptide TQHFKN (SEQ ID NO:99) or PLRADGDKPRAHLTVVRQTP (SEQ ID NO:140).

When the TNF-peptide is derived from EDA, said TNF-peptide preferably comprises, or even consists of, the peptide AVVHLQ (SEQ ID NO:100) or the peptide VVHLQG (SEQ ID NO:101), more preferably said TNF-peptide comprises, or even consists of, the peptide QPAVVHLQG (SEQ ID NO:102) or PAVVHLQGQG (SEQ ID NO:103), even more preferably it comprises, or even consists of, the peptide TRENQPAVVHLQ (SEQ ID NO:104) or ENQPAVVHLQGQGS (SEQ ID NO:105) or QPAVVHLQGQGSAI (SEQ ID NO:106) or AGTRENQPAVVHLQGQGSAI (SEQ ID NO:141).

When the TNF-peptide is derived from GITR, said TNF-peptide preferably comprises, or even consists of, the peptide CMVKF (SEQ ID NO: 107) or the peptide CMAKF (SEQ ID NO: 108), more preferably said TNF-peptide comprises, or even consists of, the peptide ESCMVKFE (SEQ ID NO:109) or EPCMAKFG (SEQ ID NO:110) or QLETAKEPCMAKFGPLPSKW (SEQ ID NO:142).

When the TNF-peptide is derived from CD30L, said TNF-peptide preferably comprises, or even consists of, the peptide WAYLQV (SEQ ID NO:111) or the peptide AAYMRV (SEQ ID NO:112), more preferably said TNF-peptide comprises, or even consists of, the peptide KGAAAYMRV (SEQ ID NO:113) or the peptide KKSWAYLQV (SEQ ID NO:114) or LKRAPFKKSWAYLQVAKHLN (SEQ ID NO:143).

When the TNF-peptide is derived from 4-1BBL, said TNF-peptide preferably comprises, or even consists of, the peptide FAQLVA (SEQ ID NO:115) or the peptide FAKLLA (SEQ ID NO:116) or the peptide LVAQNVLL (SEQ ID NO:117) or the peptide LLAKNQAS (SEQ ID NO:118) or the peptide QGMFAQLVA (SEQ ID NO:119) or DLRQGMFAQLVAQNVLL (SEQ ID NO:144).

When the TNF-peptide is derived from OX40L, said TNF-peptide preferably comprises, or even consists of, the peptide FILTSQ (SEQ ID NO:120) or the peptide FIGTSK (SEQ ID NO:121) or the peptide FILPLQ (SEQ ID NO:122), more preferably said TNF-peptide comprises, or even consists of, the peptide KGFILTSQK (SEQ ID NO:123) or the peptide RLFIGTSKK (SEQ ID NO:124) or FTEYKKEKGFILTSQKEDE (SEQ ID NO:145).

In one embodiment, the core particle comprises, or is selected from a group consisting of, a virus, a bacterial pilus, a structure formed from bacterial pilin, a bacteriophage, a virus-like particle, a virus-like particle of a RNA phage, a viral capsid particle or a recombinant form thereof. Any virus known in the art having an ordered and repetitive coat and/or core protein structure may be selected as a core particle of the invention; examples of suitable viruses include sindbis and other alphaviruses, rhabdoviruses (e.g. vesicular stomatitis virus), picornaviruses (e.g., human rhino virus, Aichi virus), togaviruses (e.g., rubella virus), orthomyxoviruses (e.g., Thogoto virus, Batken virus, fowl plague virus), polyomaviruses (e.g., polyomavirus BK, polyomavirus JC, avian polyomavirus BFDV), parvoviruses, rotaviruses, Norwalk virus, foot and mouth disease virus, a retrovirus, Hepatitis B virus, Tobacco mosaic virus, Flock House Virus, and human Papilomavirus, and preferably a RNA phage, bacteriophage Qβ, bacteriophage R17, bacteriophage M11, bacteriophage MX1, bacteriophage NL95, bacteriophage fr, bacteriophage GA, bacteriophage SP, bacteriophage MS2, bacteriophage f2, bacteriophage PP7 (for example, see Table 1 in Bachmann, M. F. and Zinkemagel, R. M., Immunol. Today 17:553-558 (1996)).

In a further embodiment, the invention utilizes genetic engineering of a virus to create a fusion between an ordered and repetitive viral envelope protein and a TNF-peptide of the invention. Alternatively, the viral envelope protein comprise a first attachment site, wherein alternatively or preferably the first attachment site is a heterologous protein, peptide, antigenic determinant or, most preferably, a reactive amino acid residue of choice. In a further embodiment, the TNF-peptide of the invention has an added second attachment site. Other genetic manipulations known to those in the art may be included in the construction of the inventive compositions; for example, it may be desirable to restrict the replication ability of the recombinant virus through genetic mutation. Furthermore, the virus used for the present invention is replication incompetent due to chemical or physical inactivation or, as indicated, due to lack of a replication competent genome. The viral protein selected for fusion to the TNF-peptide of the invention, or alternatively a first attachment site should have an organized and repetitive structure. Such an organized and repetitive structure includes paracrystalline organizations with spacings for the attachment or linkage of the TNF peptides of the invention to the surface of the virus of 3-30 nm, preferably 3-15 nm, and even more preferably of 3-8 nm. The creation of this type of fusion protein will result in multiple, ordered and repetitive TNF-peptide of the invention, or alternatively first attachment sites on the surface of the virus and reflect the normal organization of the native viral protein. As will be understood by those in the art, the first attachment site may be or be a part of any suitable protein, polypeptide, sugar, polynucleotide, peptide (amino acid), natural or synthetic polymer, a secondary metabolite or combination thereof that may serve to specifically link the TNF-peptide leading to an ordered and repetitive antigen array. Of course, direct fusions between the viral envelope protein on the TNF-peptide of the invention can be made without the introduction of first and/or second attachment sites.

In another embodiment of the invention, the core particle is a recombinant alphavirus, and more specifically, a recombinant Sinbis virus. Several members of the alphavirus family, Sindbis (Xiong, C. et al., Science 243:1188-1191 (1989); Schlesinger, S., Trends Biotechnol. 11:18-22 (1993)), Semliki Forest Virus (SFV) (Liljeström, P. & Garoff, H., Bio/Technology 9:1356-1361 (1991)) and others (Davis, N. L. et al., Virology 171:189-204 (1989)), have received considerable attention for use as virus-based expression vectors for a variety of different proteins (Lundstrom, K., Curr. Opin. Biotechnol. 8:578-582 (1997); Liljeström, P., Curr. Opin. Biotechnol. 5:495-500 (1994)) and as candidates for vaccine development. Recently, a number of patents have issued directed to the use of alphaviruses for the expression of heterologous proteins and the development of vaccines (see U.S. Pat. Nos. 5,766,602; 5,792,462; 5,739,026; 5,789,245 and 5,814,482).

Suitable host cells for viral-based core particle production are disclosed in WO 02/056905 on page 28, line 37, to page 29, line 12. Methods for introducing polynucleotide vectors into host cells are, furthermore given in WO 02/056905 on page 29, lines 13-27. Moreover, mammalian cells as recombinant host cells for the production of viral-based core particles are disclosed in WO 02/056905 on page 29, lines 28-35. The indicated paragraphs are explicitly incorporated herein by way of reference.

Further examples of RNA viruses suitable for use as core particle in the present invention include, but are not limited to, the ones disclosed in WO 03/039225 on page 32, line 5 to page 34, line 13 (paragraph 0096). Moreover, illustrative DNA viruses that may be used as core particles include, but are not limited to the ones disclosed in WO 03/039225 on page 34, line 14 to page 35, line 13 (paragraph 0097).

In other embodiments, a bacterial pilin, a subportion of a bacterial pilin, or a fusion protein which contains either a bacterial pilin or subportion thereof is used to prepare modified core particles and compositions and vaccine compositions, respectively, of the invention. Bacterial pilins may be purified from nature, or alternatively, may be recombinantly produced. Examples of pilin proteins include pilins produced by Escherichia coli, Haemophilus influenzae, Neisseria meningitidis, Neisseria gonorrhoeae, Caulobacter crescentus, Pseudomonas stutzeri, and Pseudomonas aeruginosa. The amino acid sequences of pilin proteins suitable for use with the present invention include those set out in GenBank reports AJ000636, AJ132364, AF229646, AF051814, AF051815), and X00981, the entire disclosures of which are incorporated herein by reference.

Bacterial pilin proteins are generally processed to remove N-terminal leader sequences prior to export of the proteins into the bacterial periplasm. Further, as one skilled in the art would recognize, bacterial pilin proteins used to prepare compositions and vaccine compositions, respectively, of the invention will generally not have the naturally present leader sequence.

Specific and preferred examples of pilin proteins suitable for use in the present invention are disclosed in WO 02/056905 on page 41, line 13 to line 21. Thus, one specific example of a pilin protein suitable for use in the present invention is the P-pilin of E. coli (GenBank report AF237482). An example of a Type-1 E. coli pilin suitable for use with the invention is a pilin having the amino acid sequence set out in GenBank report P04128, which is encoded by nucleic acid having the nucleotide sequence set out in GenBank report M27603. The entire disclosures of these GenBank reports are incorporated herein by reference. Again, the mature form of the above referenced protein would generally and preferably be used to prepare compositions and vaccine compositions, respectively, of the invention.

Bacterial pilins or pilin subportions suitable for use in the practice of the present invention will generally be able to associate to form ordered and repetitive antigen arrays. Accordingly, pilin mutants, including, for example, but not limited to truncations, are within the scope of the present invention.

Methods for preparing pili and pilus-like structures in vitro as well as preferred methods of modification of such pili and pilus-like structures usable for the present invention are disclosed in WO 02/056905 on page 41, line 25 to page 43, line 22.

In most instances, the pili or pilus-like structures used in compositions and vaccine compositions, respectively, of the invention will be composed of single type of a pilin subunit. Pili or pilus-like structures composed of identical subunits will generally be used.

However, the compositions of the invention also include compositions and vaccines comprising pili or pilus-like structures formed from heterogenous pilin subunits. Possible methods of expression of those preferred embodiments of the invention are disclosed in WO 02/056905 on page 43, line 28 to page 44, line 6.

The pilin proteins may be fused to the TNF-peptide of the invention. In a preferred embodiment, the at least one TNF-peptide of the invention is linked to the pili or pilus-like structure by covalent cross-linking. In a very preferred embodiment, the first attachment site is an amino group of a lysine, naturally or non-naturally occurring in pilin, and the second attachment site is a sulfhydryl group of a cysteine associated with the TNF-peptide of the invention. The first and the second attachment site are, then, linked by a hetero-bifunctional cross-linker.

Virus-like particles in the context of the present application refer to structures resembling a virus particle but which are not pathogenic. In general, virus-like particles lack the viral genome and, therefore, are noninfectious. Also, virus-like particles can be produced in large quantities by heterologous expression and can be easily purified.

In a preferred embodiment, the core particle is a virus-like particle, wherein the virus-like particle is a recombinant virus-like particle. The skilled artisan can produce VLPs using recombinant DNA technology and virus coding sequences which are readily available to the public. For example, the coding sequence of a virus envelope or core protein can be engineered for expression in a baculovirus expression vector using a commercially available baculovirus vector, under the regulatory control of a virus promoter, with appropriate modifications of the sequence to allow functional linkage of the coding sequence to the regulatory sequence. The coding sequence of a virus envelope or core protein can also be engineered for expression in a bacterial expression vector, for example.

Examples of VLPs include, but are not limited to, the capsid proteins of Hepatitis B virus (Ulrich, et al., Virus Res. 50:141-182 (1998)), measles virus (Warnes, et al., Gene 160:173-178 (1995)), Sindbis virus, rotavirus (U.S. Pat. No. 5,071,651 and U.S. Pat. No. 5,374,426), foot-and-mouth-disease virus (Twomey, et al., Vaccine 13:1603-1610, (1995)), Norwalk virus (Jiang, X., et al., Science 250:1580-1583 (1990); Matsui, S. M., et al., J. Clin. Invest. 87:1456-1461 (1991)), the retroviral GAG protein (WO 96/30523), the retrotransposon Ty protein p1, the surface protein of Hepatitis B virus (WO 92/11291), human papilloma virus (WO 98/15631), Ty and preferably RNA phages such as fr-phage, GA-phage, AP205-phage and Qβ-phage.

In a more specific embodiment, the VLP comprises, or alternatively essentially consists of, or alternatively consists of recombinant polypeptides, or fragments thereof, being selected from recombinant polypeptides of Rotavirus, recombinant polypeptides of Norwalk virus, recombinant polypeptides of Alphavirus, recombinant polypeptides of Foot and Mouth Disease virus, recombinant polypeptides of measles virus, recombinant polypeptides of Sindbis virus, recombinant polypeptides of Polyoma virus, recombinant polypeptides of Retrovirus, recombinant polypeptides of Hepatitis B virus (e.g., a HBcAg), recombinant polypeptides of Tobacco mosaic virus, recombinant polypeptides of Flock House Virus, recombinant polypeptides of human Papillomavirus, recombinant polypeptides of bacteriophages, recombinant polypeptides of RNA phages, recombinant polypeptides of Ty, recombinant polypeptides of fr-phage, recombinant polypeptides of GA-phage and recombinant polypeptides of Qβ-phage. The virus-like particle can further comprise, or alternatively essentially consist of, or alternatively consist of, one or more fragments of such polypeptides, as well as variants of such polypeptides. Variants of polypeptides can share, for example, at least 80%, 85%, 90%, 95%, 97%, or 99% identity at the amino acid level with their wild-type counterparts.

In a preferred embodiment, the virus-like particle comprises, preferably consists essentially of, or alternatively consists of recombinant proteins, or fragments thereof, of a RNA-phage. Preferably, the RNA-phage is selected from the group consisting of a) bacteriophage Qβ; b) bacteriophage R17; c) bacteriophage fr; d) bacteriophage GA; e) bacteriophage SP; f) bacteriophage MS2; g) bacteriophage M11; h) bacteriophage MX1; i) bacteriophage NL95; k) bacteriophage f2; 1) bacteriophage PP7, and m) bacteriophage AP205.

In another preferred embodiment of the present invention, the virus-like particle comprises, or alternatively consists essentially of, or alternatively consists of recombinant proteins, or fragments thereof, of the RNA-bacteriophage Qβ or of the RNA-bacteriophage fr, or of the RNA-bacteriophage AP205. In another preferred embodiment of the present invention, the virus-like particle is a VLP of a bacteriophage.

In a further preferred embodiment of the present invention, the recombinant proteins comprise, or alternatively consist essentially of, or alternatively consist of coat proteins of RNA phages.

RNA-phage coat proteins forming capsids or VLPs, or fragments of the bacteriophage coat proteins compatible with self-assembly into a capsid or a VLP, are, therefore, further preferred embodiments of the present invention. Bacteriophage Qβ coat proteins, for example, can be expressed recombinantly in E. coli. Further, upon such expression these proteins spontaneously form capsids. Additionally, these capsids form a structure with an inherent repetitive organization.

Specific preferred examples of bacteriophage coat proteins which can be used to prepare compositions of the invention include the coat proteins of RNA bacteriophages such as bacteriophage Qβ (SEQ ID NO:4; PIR Database, Accession No. VCBPQβ referring to Qβ CP and SEQ ID NO:5; Accession No. AAA16663 referring to Qβ A1 protein), bacteriophage R17 (SEQ ID NO:6; PIR Accession No. VCBPR7), bacteriophage fr (SEQ ID NO:7; PIR Accession No. VCBPFR), bacteriophage GA (SEQ ID NO:8; GenBank Accession No. NP-040754), bacteriophage SP (SEQ ID NO:9; GenBank Accession No. CAA30374 referring to SP CP and SEQ ID NO:10; Accession No. NP 695026 referring to SP A1 protein), bacteriophage MS2 (SEQ ID NO:11; PIR Accession No. VCBPM2), bacteriophage M11 (SEQ ID NO:12; GenBank Accession No. AAC06250), bacteriophage Mx1 (SEQ ID NO:13; GenBank Accession No. AAC14699), bacteriophage NL95 (SEQ ID NO:14; GenBank Accession No. AAC14704), bacteriophage f2 (SEQ ID NO:15; GenBank Accession No. P03611), bacteriophage PP7 (SEQ ID NO:16), and bacteriophage AP205 (SEQ ID NO:28). Furthermore, the A1 protein of bacteriophage Qβ (SEQ ID NO:5) or C-terminal truncated forms missing as much as 100, 150 or 180 amino acids from its C-terminus may be incorporated in a capsid assembly of Qβ coat proteins. Generally, the percentage of QβA1 protein relative to Qβ CP in the capsid assembly will be limited, in order to ensure capsid formation.

Qβ coat protein has been found to self-assemble into capsids when expressed in E. coli (Kozlovska T M. et al., GENE 137:133-137 (1993)). The obtained capsids or virus-like particle showed an icosahedral phage-like capsid structure with a diameter of 25 nm and T=3 quasi symmetry. Further, the crystal structure of phage Qβ has been solved. The capsid contains 180 copies of the coat protein, which are linked in covalent pentamers and hexamers by disulfide bridges (Golmohammadi, R. et al., Structure 4:543-5554 (1996)) leading to a remarkable stability of the capsid of Qβ coat protein. Capsids or VLPs made from recombinant Qβ coat protein may contain, however, subunits not linked via disulfide links to other subunits within the capsid, or incompletely linked. However, typically more than about 80% of the subunits are linked via disulfide bridges to each other within the VLP. Thus, upon loading recombinant Qβ capsid on non-reducing SDS-PAGE, bands corresponding to monomeric Qβ coat protein as well as bands corresponding to the hexamer or pentamer of Qβ coat protein are visible. Incompletely disulfide-linked subunits could appear as dimer, trimer or even tetramer band in non-reducing SDS-PAGE. Qβ capsid protein also shows unusual resistance to organic solvents and denaturing agents. Surprisingly, we have observed that DMSO and acetonitrile concentrations as high as 30%, and Guanidinium concentrations as high as 1 M do not affect the stability of the capsid. The high stability of the capsid of Qβ coat protein is an advantageous feature, in particular, for its use in immunization and vaccination of mammals and humans in accordance of the present invention.

Upon expression in E. coli, the N-terminal methionine of Qβ coat protein is usually removed, as we observed by N-terminal Edman sequencing as described in Stoll, E. et al, J. Biol. Chem. 252:990-993 (1977). VLP composed from Qβ coat proteins where the N-terminal methionine has not been removed, or VLPs comprising a mixture of Qβ coat proteins where the N-terminal methionine is either cleaved or present are also within the scope of the present invention.

Further preferred virus-like particles of RNA-phages, in particular of Qβ, in accordance of this invention are disclosed in WO 02/056905, the disclosure of which is herewith incorporated by reference in its entirety. In particular, a detailed description of the preparation of VLP particles from Qβ is disclosed in Example 18 of WO 02/056905.

Further RNA phage coat proteins have also been shown to self-assemble upon expression in a bacterial host (Kastelein, R A. et al., Gene 23:245-254 (1983), Kozlovskaya, T M. et al., Dokl. Akad. Nauk SSSR 287:452-455 (1986), Adhin, M R. et al., Virology 170:238-242 (1989), Ni, C Z., et al., Protein Sci. 5:2485-2493 (1996), Priano, C. et al., J. Mol. Biol. 249:283-297 (1995)). The Qβ phage capsid contains, in addition to the coat protein, the so called read-through protein A1 and the maturation protein A2. A1 is generated by suppression at the UGA stop codon and has a length of 329 aa. The capsid of phage Qβ recombinant coat protein used in the invention is devoid of the A2 lysis protein, and contains RNA from the host. The coat protein of RNA phages is an RNA binding protein, and interacts with the stem loop of the ribosomal binding site of the replicase gene acting as a translational repressor during the life cycle of the virus. The sequence and structural elements of the interaction are known (Witherell, G W. & Uhlenbeck, O C. Biochemistry 28:71-76 (1989); Lim F. et al., J. Biol. Chem. 271:31839-31845 (1996)). The stem loop and RNA in general are known to be involved in the virus assembly (Golmohammadi, R. et al., Structure 4:543-5554 (1996)).

In a further preferred embodiment of the present invention, the virus-like particle comprises, or alternatively consists essentially of, or alternatively consists of recombinant proteins, or fragments thereof, of a RNA-phage, wherein the recombinant proteins comprise, alternatively consist essentially of or alternatively consist of mutant coat proteins of a RNA phage, preferably of mutant coat proteins of the RNA phages mentioned above. In one embodiment, the mutant coat proteins are fusion proteins with a TNF-peptide of the invention. In another preferred embodiment, the mutant coat proteins of the RNA phage have been modified by removal of at least one, or alternatively at least two, lysine residue by way of substitution, or by addition of at least one lysine residue by way of substitution; alternatively, the mutant coat proteins of the RNA phage have been modified by deletion of at least one, or alternatively at least two, lysine residue, or by addition of at least one lysine residue by way of insertion. The deletion, substitution or addition of at least one lysine residue allows varying the degree of coupling, i.e. the amount of TNF peptides per subunits of the VLP of the RNA-phages, in particular, to match and tailor the requirements of the vaccine.

In another preferred embodiment, the virus-like particle comprises, or alternatively consists essentially of, or alternatively consists of recombinant proteins, or fragments thereof, of the RNA-bacteriophage Qβ, wherein the recombinant proteins comprise, or alternatively consist essentially of, or alternatively consist of coat proteins having an amino acid sequence of SEQ ID NO:4, or a mixture of coat proteins having amino acid sequences of SEQ ID NO:4 and of SEQ ID NO:5 or mutants of SEQ ID NO:5 and wherein the N-terminal methionine is preferably cleaved.

In a further preferred embodiment of the present invention, the virus-like particle comprises, consists essentially of or alternatively consists of recombinant proteins of Qβ, or fragments thereof, wherein the recombinant proteins comprise, or alternatively consist essentially of, or alternatively consist of mutant Qβ coat proteins. In another preferred embodiment, these mutant coat proteins have been modified by removal of at least one lysine residue by way of substitution, or by addition of at least one lysine residue by way of substitution. Alternatively, these mutant coat proteins have been modified by deletion of at least one lysine residue, or by addition of at least one lysine residue by way of insertion.

Four lysine residues are exposed on the surface of the capsid of Qβ coat protein. Qβ mutants, for which exposed lysine residues are replaced by arginines can also be used for the present invention. The following Qβ coat protein mutants and mutant Qβ VLPs can, thus, be used in the practice of the invention: “Qβ-240” (Lys13-Arg; SEQ ID NO:17), “Qβ-243” (Asn 10-Lys; SEQ ID NO:18), “Qβ-250” (Lys 2-Arg; Lys13-Arg; SEQ ID NO:19), “Qβ-251” (SEQ ID NO:20) and “Qβ-259” (Lys 2-Arg; Lys16-Arg; SEQ ID NO:21). Thus, in further preferred embodiment of the present invention, the virus-like particle comprises, consists essentially of or alternatively consists of recombinant proteins of mutant Qβ coat proteins, which comprise proteins having an amino acid sequence selected from the group of a) the amino acid sequence of SEQ ID NO:17; b) the amino acid sequence of SEQ ID NO:18; c) the amino acid sequence of SEQ ID NO:19; d) the amino acid sequence of SEQ ID NO:20; and e) the amino acid sequence of SEQ ID NO:21. The construction, expression and purification of the above indicated Qβ coat proteins, mutant Qβ coat protein VLPs and capsids, respectively, are described in WO 02/056905. In particular is hereby referred to Example 18 of above mentioned application.

In a further preferred embodiment of the present invention, the virus-like particle comprises, or alternatively consists essentially of, or alternatively consists of recombinant proteins of Qβ, or fragments thereof, wherein the recombinant proteins comprise, consist essentially of or alternatively consist of a mixture of either one of the foregoing Qβ mutants and the corresponding A1 protein.

In a further preferred embodiment of the present invention, the virus-like particle comprises, or alternatively essentially consists of, or alternatively consists of recombinant proteins, or fragments thereof, of RNA-phage AP205.

The AP205 genome consists of a maturation protein, a coat protein, a replicase and two open reading flames not present in related phages; a lysis gene and an open reading frame playing a role in the translation of the maturation gene (Klovins, J., et al, J. Gen. Virol. 83:1523-33 (2002)). WO 2004/007538 describes, in particular in Example 1 and Example 2, how to obtain VLP comprising AP205 coat proteins, and hereby in particular the expression and the purification thereto. WO 2004/007538, and hereby in particular the indicated Examples, are incorporated herein by way of reference. AP205 VLPs are highly immunogenic, and can be linked with TNF peptides of the invention to generate vaccine constructs displaying the TNF peptides of the invention oriented in a repetitive manner. High titers are elicited against the so displayed TNF peptides of the invention showing that bound TNF peptides of the invention are accessible for interacting with antibody molecules and are immunogenic.

In a further preferred embodiment of the present invention, the virus-like particle comprises, or alternatively essentially consists of, or alternatively consists of recombinant mutant coat proteins, or fragments thereof, of the RNA-phage AP205.

Assembly-competent mutant forms of AP205 VLPs, including AP205 coat protein with the substitution of proline at amino acid 5 to threonine may also be used in the practice of the invention and leads to further preferred embodiments of the invention. The cloning of the AP205Pro-5-Thr and the purification of the VLPs are disclosed in WO 2004/007538, and therein, in particular within Example 1 and Example 2. The disclosure of WO 2004/007538, and, in particular, Example 1 and Example 2 thereof is explicitly incorporated herein by way of reference.

In a further preferred embodiment of the present invention, the virus-like particle comprises, or alternatively essentially consists of, or alternatively consists of a mixture of recombinant coat proteins, or fragments thereof, of the RNA-phage AP205 and of recombinant mutant coat proteins, or fragments thereof, of the RNA-phage AP205.

In a further preferred embodiment of the present invention, the virus-like particle comprises, or alternatively essentially consists of, or alternatively consists of fragments of recombinant coat proteins or recombinant mutant coat proteins of the RNA-phage AP205.

Recombinant AP205 coat protein fragments capable of assembling into a VLP and a capsid, respectively are also useful in the practice of the invention. These fragments may be generated by deletion, either internally or at the termini of the coat protein and mutant coat protein, respectively. Insertions in the coat protein and mutant coat protein sequence or fusions of a TNF-peptide of the invention to the coat protein and mutant coat protein sequence, and compatible with assembly into a VLP, are further embodiments of the invention and lead to chimeric AP205 coat proteins, and particles, respectively. The outcome of insertions, deletions and fusions to the coat protein sequence and whether it is compatible with assembly into a VLP can be determined by electron microscopy.

The particles formed by the AP205 coat protein, coat protein fragments and chimeric coat proteins described above, can be isolated in pure form by a combination of fractionation steps by precipitation and of purification steps by gel filtration using e.g. Sepharose CL-4B, Sepharose CL-2B, Sepharose CL-6B columns and combinations thereof. Other methods of isolating virus-like particles are known in the art, and may be used to isolate the virus-like particles (VLPs) of bacteriophage AP205. For example, the use of ultracentrifugation to isolate VLPs of the yeast retrotransposon Ty is described in U.S. Pat. No. 4,918,166, which is incorporated by reference herein in its entirety.

The crystal structure of several RNA bacteriophages has been determined (Golmohammadi, R. et al., Structure 4:543-554 (1996)). Using such information, surface exposed residues can be identified and, thus, RNA-phage coat proteins can be modified such that one or more reactive amino acid residues can be inserted by way of insertion or substitution. As a consequence, those modified forms of bacteriophage coat proteins can also be used for the present invention. Thus, variants of proteins which form capsids or capsid-like structures (e.g., coat proteins of bacteriophage Qβ, bacteriophage R17, bacteriophage fr, bacteriophage GA, bacteriophage SP, bacteriophage AP205, and bacteriophage MS2) can also be used to prepare modified core particles and preferably modified VLPs and also compositions of the present invention.

Although the sequence of the variant proteins discussed above will differ from their wild-type counterparts, these variant proteins will generally retain the ability to form capsids or capsid-like structures. Thus, the invention further includes compositions and vaccine compositions, respectively, which further include variants of proteins which form capsids or capsid-like structures, as well as methods for preparing such compositions and vaccine compositions, respectively, individual protein subunits used to prepare such compositions, and nucleic acid molecules which encode these protein subunits. Thus, included within the scope of the invention are variant forms of wild-type proteins which form capsids or capsid-like structures and retain the ability to associate and form capsids or capsid-like structures.

As a result, the invention further includes compositions and vaccine compositions, respectively, comprising proteins, which comprise, or alternatively consist essentially of, or alternatively consist of amino acid sequences which are at least 80%, 85%, 90%, 95%, 97%, or 99% identical to wild-type proteins which form ordered arrays and having an inherent repetitive structure, respectively.

Further included within the scope of the invention are nucleic acid molecules which encode proteins used to prepare compositions of the present invention.

In other embodiments, the invention further includes compositions comprising proteins, which comprise, or alternatively consist essentially of, or alternatively consist of amino acid sequences which are at least 80%, 85%, 90%, 95%, 97%, or 99% identical to any of the amino acid sequences shown in SEQ ID NOs:4-21.

Proteins suitable for use in the present invention also include C-terminal truncation mutants of proteins which form capsids or capsid-like structures, or VLPs. Specific examples of such truncation mutants include proteins having an amino acid sequence shown in any of SEQ ID NOs:4-21 where 1, 2, 5, 7, 9, 10, 12, 14, 15, or 17 amino acids have been removed from the C-terminus. Typically, theses C-terminal truncation mutants will retain the ability to form capsids or capsid-like structures.

Further proteins suitable for use in the present invention also include N-terminal truncation mutants of proteins which form capsids or capsid-like structures. Specific examples of such truncation mutants include proteins having an amino acid sequence shown in any of SEQ ID NOs:4-21 where 1, 2, 5, 7, 9, 10, 12, 14, 15, or 17 amino acids have been removed from the N-terminus. Typically, these N-terminal truncation mutants will retain the ability to form capsids or capsid-like structures.

Additional proteins suitable for use in the present invention include N- and C-terminal truncation mutants which form capsids or capsid-like structures. Suitable truncation mutants include proteins having an amino acid sequence shown in any of SEQ ID NOs:4-21 where 1, 2, 5, 7, 9, 10, 12, 14, 15, or 17 amino acids have been removed from the N-terminus and 1, 2, 5, 7, 9, 10, 12, 14, 15, or 17 amino acids have been removed from the C-terminus. Typically, these N-terminal and C-terminal truncation mutants will retain the ability to form capsids or capsid-like structures.

The invention further includes compositions comprising proteins which comprise, or alternatively consist essentially of, or alternatively consist of, amino acid sequences which are at least 80%, 85%, 90%, 95%, 97%, or 99% identical to the above described truncation mutants.

The invention thus includes modified core particles, preferably modified VLPs, and compositions and vaccine compositions prepared from proteins which form capsids or VLPs, methods for preparing these compositions from individual protein subunits and VLPs or capsids, methods for preparing these individual protein subunits, nucleic acid molecules which encode these subunits, and methods for vaccinating and/or eliciting immunological responses in individuals using these compositions of the present invention.

In one embodiment, the invention provides a vaccine composition of the invention further comprising an adjuvant. In another embodiment, the vaccine composition of is devoid of an adjuvant. In a further embodiment of the invention, the vaccine composition comprises a core particle of the invention, wherein the core particle comprises, preferably is, a virus-like particle, wherein preferably said virus-like particle is a recombinant virus-like particle. Preferably, the virus-like particle comprises, or alternatively consists essentially of, or alternatively consists of, recombinant proteins, or fragments thereof, of a RNA-phage, preferably of coat proteins of RNA phages. In a preferred embodiment, the coat protein of the RNA phages has an amino acid are selected from the group consisting of: (a) SEQ ID NO:4; (b) a mixture of SEQ ID NO:4 and SEQ ID NO:5; (c) SEQ ID NO:6; (d) SEQ ID NO:7; (e) SEQ ID NO:8; (f) SEQ ID NO:9; (g) a mixture of SEQ ID NO:9 and SEQ ID NO:10; (h) SEQ ID NO:11; (i) SEQ ID NO:12; (k) SEQ ID NO:13; (1) SEQ ID NO:14; (m) SEQ ID NO:15; (n) SEQ ID NO:16; and (O) SEQ ID NO:28. Alternatively, the recombinant proteins of the virus-like particle of the vaccine composition of the invention comprise, or alternatively consist essentially of, or alternatively consist of mutant coat proteins of RNA phages, wherein the RNA-phage is selected from the group consisting of: (a) bacteriophage Qβ; (b) bacteriophage R17; (c) bacteriophage fr; (d) bacteriophage GA; (e) bacteriophage SP; (f) bacteriophage MS2; (g) bacteriophage M11; (h) bacteriophage MX1; (i) bacteriophage NL95; (k) bacteriophage f2; (1) bacteriophage PP7; and (m) bacteriophage AP205.

In a preferred embodiment, the mutant coat proteins of said RNA phage have been modified by removal, or by addition of at least one lysine residue by way of substitution. In another preferred embodiment, the mutant coat proteins of said RNA phage have been modified by deletion of at least one lysine residue or by addition of at least one lysine residue by way of insertion. In a preferred embodiment, the virus-like particle comprises recombinant proteins or fragments thereof, of RNA-phage Qβ, or alternatively of RNA-phage fr, or of RNA-phage AP205.

As previously stated, the invention includes virus-like particles or recombinant forms thereof. In one further preferred embodiment, the particles used in compositions of the invention are composed of a Hepatitis B core protein (HBcAg) or a fragment of a HBcAg. In a further embodiment, the particles used in compositions of the invention are composed of a Hepatitis B core protein (HBcAg) or a fragment of a HBcAg protein, which has been modified to either eliminate or reduce the number of free cysteine residues. Zhou et al. (J. Virol. 66:5393-5398 (1992)) demonstrated that HBcAgs which have been modified to remove the naturally resident cysteine residues retain the ability to associate and form capsids. Thus, VLPs suitable for use in compositions of the invention include those comprising modified HBcAgs, or fragments thereof, in which one or more of the naturally resident cysteine residues have been either deleted or substituted with another amino acid residue (e.g., a serine residue).

The HBcAg is a protein generated by the processing of a Hepatitis B core antigen precursor protein. A number of isotypes of the HBcAg have been identified and their amino acids sequences are readily available to those skilled in the art. In most instances, compositions and vaccine compositions, respectively, of the invention will be prepared using the processed form of a HBcAg (i.e., an HBcAg from which the N-terminal leader sequence of the Hepatitis B core antigen precursor protein has been removed).

Further, when HBcAgs are produced under conditions where processing will not occur, the HBcAgs will generally be expressed in “processed” form. For example, when an E. coli expression system directing expression of the protein to the cytoplasm is used to produce HBcAgs of the invention, these proteins will generally be expressed such that the N-terminal leader sequence of the Hepatitis B core antigen precursor protein is not present.

The preparation of Hepatitis B virus-like particles, which can be used for the present invention, is disclosed, for example, in WO 00/32227, and hereby in particular in Examples 17 to 19 and 21 to 24, as well as in WO 01/85208, and hereby in particular in Examples 17 to 19, 21 to 24, 31 and 41, and in WO 02/056905. For the latter application, it is in particular referred to Example 23, 24, 31 and 51. All three documents are explicitly incorporated herein by reference.

The present invention also includes HBcAg variants which have been modified to delete or substitute one or more additional cysteine residues. It is known in the art that free cysteine residues can be involved in a number of chemical side reactions. These side reactions include disulfide exchanges, reaction with chemical substances or metabolites that are, for example, injected or formed in a combination therapy with other substances, or direct oxidation and reaction with nucleotides upon exposure to UV light. Toxic adducts could thus be generated, especially considering the fact that HBcAgs have a strong tendency to bind nucleic acids. The toxic adducts would thus be distributed between a multiplicity of species, which individually may each be present at low concentration, but reach toxic levels when together.

In view of the above, one advantage to the use of HBcAgs in vaccine compositions which have been modified to remove naturally resident cysteine residues is that sites to which toxic species can bind when antigens or antigenic determinants are attached would be reduced in number or eliminated altogether.

A number of naturally occurring HBcAg variants suitable for use in the practice of the present invention has been identified. The amino acid sequences of a number of HBcAg variants, as well as several Hepatitis B core antigen precursor variants, are disclosed in GenBank reports AAF121240, AF121239, X85297, X02496, X85305, X85303, AF151735, X85259, X85286, X85260, X85317, X85298, AF043593, M20706, X85295, X80925, X85284, X85275, X72702, X85291, X65258, X85302, M32138, X85293, X85315, U95551, X85256, X85316, X85296, AB033559, X59795, X85299, X85307, X65257, X85311, X85301, X85314, X85287, X85272, X85319, AB010289, X85285, AB010289, AF121242, M90520, P03153, AF110999, and M95589, the disclosures of each of which are incorporated herein by reference. The sequences of the hereinabove mentioned Hepatitis B core antigen precursor variants are further disclosed in WO 01/85208 in SEQ ID NOs:89-138. Further HBcAg variants suitable for use in the compositions of the invention, and which may be further modified according to the disclosure of this specification are described in WO 00/198333, WO 00/177158 and WO 00/214478.

In a further preferred embodiment, the virus-like particle comprises, or alternatively consists essentially of, or alternatively consists of recombinant proteins of SEQ ID NO:25.

Whether the amino acid sequence of a polypeptide has an amino acid sequence that is at least 80%, 85%, 90%, 95%, 97% or 99% identical to one of the above amino acid sequences, or a subportion thereof, can be determined conventionally using known computer programs such the Bestfit program. When using Bestfit or any other sequence alignment program to determine whether a particular sequence is, for instance, 95% identical to a reference amino acid sequence, the parameters are set such that the percentage of identity is calculated over the full length of the reference amino acid sequence and that gaps in homology of up to 5% of the total number of amino acid residues in the reference sequence are allowed.

The amino acid sequences of the hereinabove mentioned HBcAg variants and precursors are relatively similar to each other. Thus, reference to an amino acid residue of a HBcAg variant located at a position which corresponds to a particular position in SEQ ID NO:25, refers to the amino acid residue which is present at that position in the amino acid sequence shown in SEQ ID NO:25. The homology between these HBcAg variants is for the most part high enough among Hepatitis B viruses that infect mammals so that one skilled in the art would have little difficulty reviewing both the amino acid sequence shown in SEQ ID NO:25 and that of a particular HBcAg variant and identifying “corresponding” amino acid residues.

The invention also includes vaccine compositions which comprise HBcAg variants of Hepatitis B viruses which infect birds, as wells as vaccine compositions which comprise fragments of these HBcAg variants. For these HBcAg variants one, two, three or more of the cysteine residues naturally present in these polypeptides could be either substituted with another amino acid residue or deleted prior to their inclusion in vaccine compositions of the invention.

As discussed above, the elimination of free cysteine residues reduces the number of sites where toxic components can bind to the HBcAg, and also eliminates sites where cross-linking of lysine and cysteine residues of the same or of neighboring HBcAg molecules can occur. Therefore, in another embodiment of the present invention, one or more cysteine residues of the Hepatitis B virus capsid protein have been either deleted or substituted with another amino acid residue.

In other embodiments, compositions and vaccine compositions, respectively, of the invention will contain HBcAgs from which the C-terminal region (e.g., amino acid residues 145-185 or 150-185 of SEQ ID NO:25) has been removed. Thus, additional modified HBcAgs suitable for use in the practice of the present invention include C-terminal truncation mutants. Suitable truncation mutants include HBcAgs where 1, 5, 10, 15, 20, 25, 30, 34, 35, amino acids have been removed from the C-terminus.

HBcAgs suitable for use in the practice of the present invention also include N-terminal truncation mutants. Suitable truncation mutants include modified HBcAgs where 1, 2, 5, 7, 9, 10, 12, 14, 15, or 17 amino acids have been removed from the N-terminus.

Further HBcAgs suitable for use in the practice of the present invention include N- and C-terminal truncation mutants. Suitable truncation mutants include HBcAgs where 1, 2, 5, 7, 9, 10, 12, 14, 15, and 17 amino acids have been removed from the N-terminus and 1, 5, 10, 15, 20, 25, 30, 34, 35 amino acids have been removed from the C-terminus.

The invention further includes compositions and vaccine compositions, respectively, comprising HBcAg polypeptides comprising, or alternatively essentially consisting of, or alternatively consisting of, amino acid sequences which are at least 80%, 85%, 90%, 95%, 97%, or 99% identical to the above described truncation mutants.

In certain embodiments of the invention, a lysine residue is introduced into a HBcAg polypeptide, to mediate the binding of TNF-peptide of the invention to the VLP of HBcAg. In preferred embodiments, modified core particles, and in particular modified VLPs of the invention, and compositions of the invention are prepared using a HBcAg comprising, or alternatively consisting of, amino acids 1-144, or 1-149, 1-185 of SEQ ID NO:25, which is modified so that the amino acids corresponding to positions 79 and 80 are replaced with a peptide having the amino acid sequence of Gly-Gly-Lys-Gly-Gly (SEQ ID NO:27) resulting in the HBcAg polypeptide having the sequence shown in SEQ ID NO:26). In further preferred embodiments, the cysteine residues at positions 48 and 107 of SEQ ID NO:25 are mutated to serine. The invention further includes compositions comprising the corresponding polypeptides having amino acid sequences shown in any of the hereinabove mentioned Hepatitis B core antigen precursor variants, which also have above noted amino acid alterations. Further included within the scope of the invention are additional HBcAg variants which are capable of associating to form a capsid or VLP and have the above noted amino acid alterations. Thus, the invention further includes compositions and vaccine compositions, respectively, comprising HBcAg polypeptides which comprise, or alternatively consist of, amino acid sequences which are at least 80%, 85%, 90%, 95%, 97% or 99% identical to any of the wild-type amino acid sequences, and forms of these proteins which have been processed, where appropriate, to remove the N-terminal leader sequence and modified with above noted alterations.

Compositions or vaccine compositions of the invention may comprise mixtures of different HBcAgs. Thus, these vaccine compositions may be composed of HBcAgs which differ in amino acid sequence. For example, vaccine compositions could be prepared comprising a “wild-type” HBcAg and a modified HBcAg in which one or more amino acid residues have been altered (e.g., deleted, inserted or substituted). Further, preferred vaccine compositions of the invention are those which present highly ordered and repetitive antigen array, wherein the antigen is a TNF-peptide of the invention.

In a further preferred embodiment of the present invention, the at least one TNF-peptide of the invention is bound to said core particle and virus-like particle, respectively, by at least one covalent bond. Preferably, the at least one TNF-peptide is bound to the core particle and virus-like particle, respectively, by at least one covalent bond, said covalent bond being a non-peptide bond leading to a core particle-TNF peptide array or conjugate, which is typically and preferably an ordered and repetitive array or conjugate. This TNF-peptide-VLP array and conjugate, respectively, has typically and preferably a repetitive and ordered structure since the at least one, but usually more than one, TNF-peptide of the invention is bound to the VLP and core particle, respectively, in an oriented manner. Preferably, more than 120, preferably more than 180, more preferably more than 270, and even more preferably more than 360 TNF-peptides of the invention are bound to the VLP. The formation of a repetitive and ordered TNF-VLP and core particle, respectively, array and conjugate, respectively, is ensured by an oriented and directed as well as defined binding and attachment, respectively, of the at least one TNF-peptide of the invention to the VLP and core particle, respectively, as will become apparent in the following. Furthermore, the typical inherent highly repetitive and organized structure of the VLPs and core particles, respectively, advantageously contributes to the ability to display the TNF-peptide of the invention in a preferably highly ordered and repetitive fashion leading to a highly organized and repetitive TNF-peptide-VLP/core particle array and conjugate, respectively.

In a further preferred embodiment of the present invention, the core particle or the virus-like particle comprises at least one first attachment site and wherein said at least one TNF-peptide further comprises at least one second attachment site being selected from the group consisting of (i) an attachment site not naturally occurring with the at least one TNF-peptide; and (ii) an attachment site naturally occurring with the at least one TNF-peptide, and wherein said binding of the TNF-peptide to the core particle or the virus-like particle is effected through association between the first attachment site and the second attachment site, and wherein preferably the association is through at least one non-peptide bond.

In again a further preferred embodiment of the present invention, the modified VLP comprises said VLP with at least one first attachment site, and further, the modified VLP comprises said TNF peptide with at least one second attachment site being selected from the group consisting of (i) an attachment site not naturally occurring with the at least one TNF-peptide; and (ii) an attachment site naturally occurring with the at least one TNF-peptide, and wherein the second attachment site is capable of association to the first attachment site; and wherein preferably the TNF peptide and the VLP interact through said association to form an ordered and repetitive antigen array. Preferably, the association is through at least one non-peptide bond.

The present invention discloses methods of binding of the at least one TNF-peptide of the invention to core particles and VLPs, respectively. As indicated, in one preferred aspect of the invention, the TNF-peptide of the invention is bound to the core particle and VLP, respectively, by way of chemical cross-linking, typically and preferably by using a heterobifunctional cross-linker. Several hetero-bifunctional cross-linkers are known in the art. In preferred embodiments, the hetero-bifunctional cross-linker contains a functional group which can react with preferred first attachment sites, i.e. with the side-chain amino group of lysine residues of the core particle and the VLP or at least one VLP subunit, respectively, and a further functional group which can react with a preferred second attachment site, i.e. a cysteine residue added to or engineered to be added to the TNF-peptide of the invention, and optionally also made available for reaction by reduction. The first step of the procedure, typically called the derivatization, is the reaction of the core particle or the VLP with the cross-linker. The product of this reaction is an activated core particle or activated VLP, also called activated carrier. In the second step, unreacted cross-linker is removed using usual methods such as gel filtration or dialysis. In the third step, the TNF-peptide of the invention is reacted with the activated carrier, and this step is typically called the coupling step. Unreacted TNF-peptide of the invention may be optionally removed in a fourth step, for example by dialysis. Several hetero-bifunctional cross-linkers are known to the art. These include the preferred cross-linkers SMPH (Pierce), Sulfo-MBS, Sulfo-EMCS, Sulfo-GMBS, Sulfo-SIAB, Sulfo-SMPB, Sulfo-SMCC, SVSB, SIA and other cross-linkers available for example from the Pierce Chemical Company (Rockford, Ill., USA), and having one functional group reactive towards amino groups and one functional group reactive towards cysteine residues. The above mentioned cross-linkers all lead to formation of an amide bond after reaction with the amino group and a thioether linkage with the cysteine. Another class of cross-linkers suitable in the practice of the invention is characterized by the introduction of a disulfide linkage between the TNF-peptide of the invention and the core particle or VLP upon coupling. Preferred cross-linkers belonging to this class include for example SPDP and Sulfo-LC-SPDP (Pierce). The extent of derivatization of the core particle and VLP, respectively, with cross-linker can be influenced by varying experimental conditions such as the concentration of each of the reaction partners, the excess of one reagent over the other, the pH, the temperature and the ionic strength. The degree of coupling, i.e. the amount of TNF-peptides of the invention per subunits of the core particle and VLP, respectively, can be adjusted by varying the experimental conditions described above to match the requirements of the vaccine. Solubility of the TNF-peptide of the invention may impose a limitation on the amount of TNF-peptide of the invention that can be coupled on each subunit, and in those cases where the obtained vaccine would be insoluble reducing the amount of TNF-peptide of the invention per subunit is beneficial.

A particularly favored method of binding of TNF-peptide of the invention to the core particle and the VLP, respectively, is the linking of a lysine residue on the surface of the core particle and the VLP, respectively, with a cysteine residue on the TNF-peptide of the invention. Thus, in a preferred embodiment of the present invention, the first attachment site is a lysine residue and the second attachment site is a cysteine residue. In some embodiments, engineering of an amino acid linker containing a cysteine residue, as a second attachment site or as a part thereof, to the TNF-peptide of the invention for coupling to the core particle and VLP, respectively, may be required. Alternatively, a cysteine may be introduced by addition to the TNF-peptide of the invention. Alternatively, the cysteine residue may be introduced by chemical coupling.

In a further preferred embodiment of the present invention, the at least one first attachment site comprises, or preferably is, an amino group, and wherein even further preferably the first attachment site is an amino group of a lysine residue.

In another preferred embodiment of the present invention, the at least one second attachment site comprises, or preferably is, a sulfhydryl group, and wherein even further preferably the second attachment site is a sulfhydryl group of a cysteine residue.

In an even further preferred embodiment of the present invention, the first attachment site is not, and preferably does not comprise, a sulfhydryl group, and wherein further preferably the first attachment site is not, and again preferably does not comprise, a sulfhydryl group of a cysteine residue.

The selection of the amino acid linker will be dependent on the nature of the TNF-peptide of the invention, on its biochemical properties, such as pI, charge distribution and glycosylation. Typically, flexible amino acid linkers are favored. Preferred embodiments of the amino acid linker are disclosed in WO 03/039225 on page 60, line 24 to page 61, line 11 (paragraphs 00179 and 00180), which are explicitly incorporated herein by way of reference.

In a further preferred embodiment of the present invention, and in particular if the TNF-peptide of the invention is derived from RANKL or TNFα, preferred amino acid linkers are GGCG (SEQ ID NO:24), GGC or GGC-NH2 (“NH2” stands for amidation) linkers at the C-terminus of the peptide or CGG at its N-terminus. In general, glycine residues will be inserted between bulky amino acids and the cysteine to be used as second attachment site, to avoid potential steric hindrance of the bulkier amino acid in the coupling reaction.

The cysteine residue added to the TNF-peptide of the invention has to be in its reduced state to react with the hetero-bifunctional cross-linker on the activated VLP, that is a free cysteine or a cysteine residue with a free sulfhydryl group has to be available. In the instance where the cysteine residue to function as binding site is in an oxidized form, for example if it is forming a disulfide bridge, reduction of this disulfide bridge with e.g. DTT, TCEP or β-mercaptoethanol is required.

Binding of the TNF-peptide of the invention to the core particle and VLP, respectively, by using a hetero-bifunctional cross-linker according to the preferred methods described above, allows coupling of the TNF-peptide of the invention to the core particle and the VLP, respectively, in an oriented fashion. Other methods of binding the TNF-peptide of the invention to the core particle and the VLP, respectively, include methods wherein the TNF-peptide of the invention is cross-linked to the core particle and the VLP, respectively, using the carbodiimide EDC, and NHS. The TNF-peptide of the invention may also be first thiolated through reaction, for example with SATA, SATP or iminothiolane. The TNF-peptide of the invention, after deprotection if required, may then be coupled to the core particle and the VLP, respectively, as follows. After separation of the excess thiolation reagent, the TNF-peptide of the invention is reacted with the core particle and the VLP, respectively, previously activated with a hetero-bifunctional cross-linker comprising a cysteine reactive moiety, and therefore displaying at least one or several functional groups, preferably one, reactive towards cysteine residues, to which the thiolated TNF-peptide of the invention can react, such as described above. Optionally, low amounts of a reducing agent are included in the reaction mixture. In further methods, the TNF-peptide of the invention is attached to the core particle and the VLP, respectively, using a homo-bifunctional cross-linker such as glutaraldehyde, DSG, BM[PEO]₄, BS³, (Pierce Chemical Company, Rockford, Ill., USA) or other known homo-bifunctional cross-linkers with functional groups reactive towards amine groups or carboxyl groups of the core particle and the VLP, respectively.

Other methods of binding the VLP to a TNF-peptide of the invention include methods where the core particle and the VLP, respectively, is biotinylated, and the TNF-peptide of the invention expressed as a streptavidin-fusion protein, or methods wherein both the TNF-peptides of the invention and the core particle and the VLP, respectively, are biotinylated, for example as described in WO 00/23955. In this case, the TNF-peptide of the invention may be first bound to streptavidin or avidin by adjusting the ratio of TNF-peptide of the invention to streptavidin such that free binding sites are still available for binding of the core particle and the VLP, respectively, which is added in the next step. Alternatively, all components may be mixed in a “one pot” reaction. Other ligand-receptor pairs, where a soluble form of the receptor and of the ligand is available, and are capable of being cross-linked to the core particle and the VLP, respectively, or the TNF-peptide of the invention, may be used as binding agents for binding the TNF-peptide of the invention to the core particle and the VLP, respectively. Alternatively, either the ligand or the receptor may be fused to the TNF-peptide of the invention, and so mediate binding to the core particle and the VLP, respectively, chemically bound or fused either to the receptor, or the ligand respectively. Fusion may also be effected by insertion or substitution.

As already indicated, in a favored embodiment of the present invention, the VLP is the VLP of a RNA phage, and in a more preferred embodiment, the VLP is the VLP of RNA phage Qβ coat protein.

One or several antigen molecules, i.e. TNF-peptides of the invention, can be attached to one subunit of the capsid or VLP of RNA phages coat proteins, preferably through the exposed lysine residues of the VLP of RNA phages, if sterically allowable. A specific feature of the VLP of the coat protein of RNA phages and in particular of the Qβ coat protein VLP is thus the possibility to couple several antigens per subunit. This allows for the generation of a dense antigen array.

In a preferred embodiment of the invention, the binding and attachment, respectively, of the at least one TNF-peptide of the invention to the core particle and the virus-like particle, respectively, is by way of interaction and association, respectively, between at least one first attachment site of the virus-like particle and at least one second attachment added to the TNF-peptide of the invention.

VLPs or capsids of Qβ coat protein display a defined number of lysine residues on their surface, with a defined topology with three lysine residues pointing towards the interior of the capsid and interacting with the RNA, and four other lysine residues exposed to the exterior of the capsid. These defined properties favor the attachment of antigens to the exterior of the particle, rather than to the interior of the particle where the lysine residues interact with RNA. VLPs of other RNA phage coat proteins also have a defined number of lysine residues on their surface and a defined topology of these lysine residues.

In further preferred embodiments of the present invention, the first attachment site is a lysine residue and/or the second attachment comprises sulfhydryl group or a cysteine residue. In a very preferred embodiment of the present invention, the first attachment site is a lysine residue and the second attachment is a cysteine residue.

In very preferred embodiments of the invention, the TNF-peptide of the invention is bound via a cysteine residue, having been added to the TNF-peptide of the invention, to lysine residues of the VLP of RNA phage coat protein, and in particular to the VLP of Qβ coat protein.

Another advantage of the VLPs derived from RNA phages is their high expression yield in bacteria that allows production of large quantities of material at affordable cost. Another preferred embodiment are VLPs derived from fusion proteins of RNA phage coat proteins with a TNF-polypeptide of the invention.

The use of the VLPs as carriers allows the formation of robust antigen arrays and conjugates, respectively, with variable antigen density. In particular, the use of VLPs of RNA phages, and hereby in particular the use of the VLP of RNA phage Qβ coat protein allows achievement of a very high epitope or antigen density. The preparation of compositions of VLPs of RNA phage coat proteins with a high epitope or antigen density can be effected by using the teaching of this application. In a preferred embodiment, the compositions and vaccines of the invention have an antigen density being from 0.05 to 4.0. The term “antigen density”, as used herein, refers to the average number of TNF-peptide of the invention which is linked per subunit, preferably per coat protein, of the VLP, and hereby preferably of the VLP of a RNA phage. Thus, this value is calculated as an average over all the subunits or monomers of the VLP, preferably of the VLP of the RNA-phage, in the composition or vaccines of the invention. In a further preferred embodiment of the invention, the antigen density is, preferably between 0.1 and 4.0.

As described above, four lysine residues are exposed on the surface of the VLP of Qβ coat protein. Typically these residues are derivatized upon reaction with a cross-linker molecule. In the instance where not all of the exposed lysine residues can be coupled to an antigen, the lysine residues which have reacted with the cross-linker are left with a cross-linker molecule attached to the ε-amino group after the derivatization step. This leads to disappearance of one or several positive charges, which may be detrimental to the solubility and stability of the VLP. By replacing some of the lysine residues with arginines, as in the disclosed Qβ coat protein mutants described below, we prevent the excessive disappearance of positive charges since the arginine residues do not react with the preferred cross-linkers. Moreover, replacement of lysine residues by arginines may lead to more defined antigen arrays, as fewer sites are available for reaction to the antigen.

Accordingly, exposed lysine residues were replaced by arginines in the following Qβ coat protein mutants and mutant Qβ VLPs. Thus, in another preferred embodiment of the present invention, the virus-like particle comprises, consists essentially of or alternatively consists of mutant Qβ coat proteins. Preferably these mutant coat proteins comprise or alternatively consist of an amino acid sequence selected from the group of a) Qβ-240 (Lys13-Arg; SEQ ID NO:17) b) Qβ-243 (Asn 10-Lys; SEQ ID NO:18), c) Qβ-250 (Lys2-Arg of SEQ ID NO:19) d) Qβ-251 (Lys16-Arg of SEQ ID NO:20); and e) Qβ-259” (Lys2-Arg; Lys16-Arg of SEQ ID NO:21). The construction, expression and purification of the above indicated Qβ coat proteins, mutant Qβ coat protein VLPs and capsids, respectively, are described in WO 02/056905. In particular is hereby referred to Example 18 of above mentioned application. In another preferred embodiment of the present invention, the virus-like particle comprises, or alternatively consists essentially of, or alternatively consists of recombinant proteins of Qβ, or fragments thereof, wherein the recombinant proteins comprise, consist essentially of or alternatively consist of a mixture of either one of the foregoing mutants and the corresponding A1 protein.

A particularly favored method of attachment of antigens to VLPs, and in particular to VLPs of RNA phage coat proteins is the linking of a lysine residue present on the surface of the VLP of RNA phage coat proteins with a cysteine residue naturally present or engineered on the antigen, i.e. the TNF-peptide of the invention. In order for a cysteine residue to be effective as second attachment site, a sulfhydryl group must be available for coupling. Thus, a cysteine residue has to be in its reduced state, that is, a free cysteine or a cysteine residue with a free sulfhydryl group has to be available. In the instant where the cysteine residue to function as second attachment site is in an oxidized form, for example if it is forming a disulfide bridge, reduction of this disulfide bridge with e.g. DTT, TCEP or β-mercaptoethanol is required. The concentration of reductand, and the molar excess of reductant over antigen have to be adjusted for each antigen. A titration range, starting from concentrations as low as 10 μM or lower, up to 10 to 20 mM or higher reductant if required is tested, and coupling of the antigen to the carrier assessed. Although low concentrations of reductant are compatible with the coupling reaction as described in WO 02/056905, higher concentrations inhibit the coupling reaction, as a skilled artisan would know, in which case the reductant has to be removed by dialysis or gel filtration. Advantageously, the pH of the dialysis or equilibration buffer is lower than 7, preferably 6. The compatibility of the low pH buffer with antigen activity or stability has to be tested.

Epitope density on the VLP of RNA phage coat proteins can be modulated by the choice of cross-linker and other reaction conditions. For example, the cross-linkers Sulfo-GMBS and SMPH typically allow reaching high epitope density. Derivatization is positively influenced by high concentration of reactands, and manipulation of the reaction conditions can be used to control the number of antigens coupled to VLPs of RNA phage coat proteins, and in particular to VLPs of Qβ coat protein.

Prior to the design of a non-natural second attachment site the position at which it should be fused, inserted or generally engineered has to be chosen. Thus, the location of the second attachment site will be selected such that steric hindrance from the second attachment site or any amino acid linker containing the same is avoided. In further embodiments, an antibody response directed at a site distinct from the interaction site of the self-antigen with its natural ligand is desired. In such embodiments, the second attachment site may be selected such that it prevents generation of antibodies against the interaction site of the self-antigen with its natural ligands.

In preferred embodiments, the TNF-peptide of the invention comprises an added single second attachment site or a single reactive attachment site capable of association with the first attachment sites on the core particle and the VLPs or VLP subunits, respectively. This ensures a defined and uniform binding and association, respectively, of the at least one, but typically more than one, preferably more than 10, 20, 40, 80, 120, 150, 180, 210, 240, 270, 300, 360, 400, 450 TNF-peptides of the invention to the core particle and VLP, respectively. The provision of a single second attachment site or a single reactive attachment site on the antigen, thus, ensures a single and uniform type of binding and association, respectively leading to a very highly ordered and repetitive array. For example, if the binding and association, respectively, is effected by way of a lysine- (as the first attachment site) and cysteine- (as a second attachment site) interaction, it is ensured, in accordance with this preferred embodiment of the invention, that only one added cysteine residue per TNF-peptide of the invention is capable of binding and associating, respectively, with the VLP and the first attachment site of the core particle, respectively.

In some embodiments, engineering of a second attachment site onto the TNF-peptide of the invention is achieved by the fusion of an amino acid linker containing an amino acid suitable as second attachment site according to the disclosures of this invention. Therefore, in a preferred embodiment of the present invention, an amino acid linker is bound to the TNF-peptide, preferably, by way of at least one covalent bond. Preferably, the amino acid linker comprises, or alternatively consists of, the second attachment site. In a further preferred embodiment, the amino acid linker comprises a sulfhydryl group or a cysteine residue. In another preferred embodiment, the amino acid linker is cysteine. Some criteria of selection of the amino acid linker as well as further preferred embodiments of the amino acid linker according to the invention have already mentioned above.

In a further preferred embodiment of the invention, the at least one TNF-peptide of the invention is fused to the core particle and the virus-like particle, respectively. As outlined above, a VLP is typically composed of at least one subunit assembling into a VLP. Thus, in again a further preferred embodiment of the invention, the TNF-peptide of the invention is fused to at least one subunit of the virus-like particle or of a protein capable of being incorporated into a VLP generating a chimeric VLP-subunit TNF-peptide protein fusion.

Fusion of TNF-peptides of the invention can be effected by insertion into the VLP subunit sequence, or by fusion to either the N- or C-terminus of the VLP-subunit or protein capable of being incorporated into a VLP. Hereinafter, when referring to fusion proteins of a peptide to a VLP subunit, the fusion to either ends of the subunit sequence or internal insertion of the peptide within the subunit sequence are encompassed, the fusion with the TNF-peptide of the invention being at the N-terminus of the fusion polypeptide, i.e. fused via its C-terminus to the VLP subunit.

Fusion may also be effected by inserting sequences of the TNF-peptide of the invention into a variant of a VLP subunit where part of the subunit sequence has been deleted, that are further referred to as truncation mutants. Truncation mutants may have N- or C-terminal, or internal deletions of part of the sequence of the VLP subunit. For example, the specific VLP HBcAg with, for example, deletion of amino acid residues 79 to 81 is a truncation mutant with an internal deletion. Fusion of TNF-peptide of the invention to either the N- or C-terminus of the truncation mutants VLP-subunits also lead to embodiments of the invention. Likewise, fusion of an epitope into the sequence of the VLP subunit may also be effected by substitution, where for example for the specific VLP HBcAg, amino acids 79-81 are replaced with a foreign epitope. Thus, fusion, as referred to hereinafter, may be effected by insertion of the sequence of the TNF-peptide of the invention into the sequence of a VLP subunit, by substitution of part of the sequence of the VLP subunit with the sequence of the TNF-peptide of the invention, or by a combination of deletion, substitution or insertions.

The chimeric TNF-peptide-VLP subunit in general will be capable of self-assembly into a VLP. VLP displaying epitopes fused to their subunits are also herein referred to as chimeric VLPs. As indicated, the virus-like particle comprises or alternatively is composed of at least one VLP subunit. In a further embodiment of the invention, the virus-like particle comprises or alternatively is composed of a mixture of chimeric VLP subunits and non-chimeric VLP subunits, i.e. VLP subunits not having an antigen fused thereto, leading to so called mosaic particles. This may be advantageous to ensure formation of and assembly to a VLP. In those embodiments, the proportion of chimeric VLP-subunits of total VLP subunits may be 1, 2, 5, 10, 20, 30, 40, 50, 60, 70, 80, 90, 95% or higher.

Flanking amino acid residues may be added to either end of the sequence of the TNF-peptide of the invention, fulfilling the requirements as set out for fusion polypeptides of the invention above, to be fused to either end of the sequence of the subunit of a VLP, or for internal insertion of such peptidic sequence into the sequence of the subunit of a VLP. Glycine and serine residues are particularly favored amino acids to be used in the flanking sequences added to the TNF-peptide of the invention to be fused. Glycine residues confer additional flexibility, which may diminish the potentially destabilizing effect of fusing a foreign sequence into the sequence of a VLP subunit.

In a specific embodiment of the invention, the VLP is a Hepatitis B core antigen VLP. Fusion proteins to either the N-terminus of HBcAg (Neyrinck, S. et al., Nature Med. 5:1157-1163 (1999)) or insertions in the so called major immunodominant region (MIR) have been described (Pumpens, P. and Grens, E., Intervirology 44:98-114 (2001)), WO 01/98333), and are preferred embodiments of the invention. Naturally occurring variants of HBcAg with deletions in the MIR have also been described (Pumpens, P. and Grens, E., Intervirology 44:98-114 (2001), which is expressly incorporated by reference in their entirety), and fusions to the N- or C-terminus, as well as insertions at the position of the MIR corresponding to the site of deletion as compared to a wt HBcAg are further embodiments of the invention. Fusions to the C-terminus have also been described (Pumpens, P. and Grens, E., Intervirology 44:98-114 (2001)). One skilled in the art will easily find guidance on how to construct fusion proteins using classical molecular biology techniques (Sambrook, J. et al., eds., Molecular Cloning, A Laboratory Manual, 2nd. edition, Cold Spring Harbor Laboratory Press, Cold Spring Harbor, N.Y. (1989), Ho et al., Gene 77:51 (1989)).

In a further preferred embodiment of the invention, the VLP is a VLP of a RNA phage. The major coat proteins of RNA phages spontaneously assemble into VLPs upon expression in bacteria, and in particular in E. coli. Specific examples of bacteriophage coat proteins which can be used to prepare compositions of the invention include the coat proteins of RNA bacteriophages such as bacteriophage Qβ (SEQ ID NO:4; PIR Database, Accession No. VCBPQβ referring to Qβ CP and SEQ ID NO:5; Accession No. AAA16663 referring to Qβ A1 protein) and bacteriophage fr (SEQ ID NO:7; PIR Accession No. VCBPFR).

In a more preferred embodiment, the at least one TNF-peptide of the invention is fused to a Qβ coat protein. Fusion protein constructs wherein epitopes have been fused to the C-terminus of a truncated form of the A1 protein of Qβ, or inserted within the A1 protein have been described (Kozlovska, T. M., et al., Intervirology, 39:9-15 (1996)). The A1 protein is generated by suppression at the UGA stop codon and has a length of 329 aa, or 328 aa, if the cleavage of the N-terminal methionine is taken into account. Cleavage of the N-terminal methionine before an alanine (the second amino acid encoded by the Qβ CP gene) usually takes place in E. coli, and such is the case for N-termini of the Qβ coat proteins CP. The part of the A1 gene, 3′ of the UGA amber codon encodes the CP extension, which has a length of 195 amino acids. Insertion of the at least one TNF-peptide of the invention between position 72 and 73 of the CP extension leads to further embodiments of the invention (Kozlovska, T. M., et al., Intervirology 39:9-15 (1996)). Fusion of a TNF-peptide of the invention at the C-terminus of a C-terminally truncated Qβ A1 protein leads to further preferred embodiments of the invention. For example, Kozlovska et al., (Intervirology, 39: 9-15 (1996)) describe Qβ A1 protein fusions where the epitope is fused at the C-terminus of the Qβ CP extension truncated at position 19.

As described by Kozlovska et al. (Intervirology, 39:9-15 (1996)), assembly of the particles displaying the fused epitopes typically requires the presence of both the A1 protein-TNF-peptide fusion and the wt CP to form a mosaic particle. However, embodiments comprising virus-like particles, and hereby in particular the VLPs of the RNA phage Qβ coat protein, which are exclusively composed of VLP subunits having at least one TNF-peptide of the invention fused thereto, are also within the scope of the present invention.

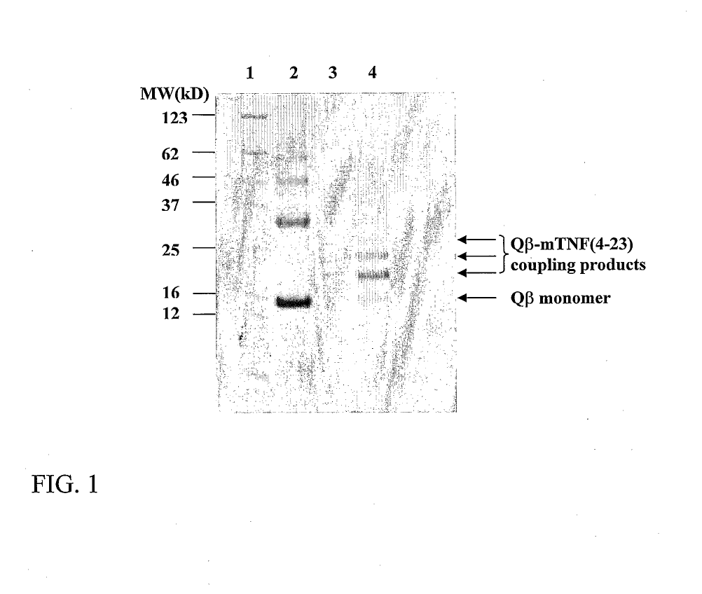

The production of mosaic particles may be effected in a number of ways. Kozlovska et al., Intervirolog, 39:9-15 (1996), describe two methods, which both can be used in the practice of the invention. In the first approach, efficient display of the fused epitope on the VLPs is mediated by the expression of the plasmid encoding the Qβ A1 protein fusion having a UGA stop codong between CP and CP extension in a E. coli strain harboring a plasmid encoding a cloned UGA suppressor tRNA which leads to translation of the UGA codon into Trp (pISM3001 plasmid (Smiley B. K., et al., Gene 134:33-40 (1993))). In another approach, the CP gene stop codon is modified into UAA, and a second plasmid expressing the A1 protein-TNF-peptide fusion is cotransformed. The second plasmid encodes a different antibiotic resistance and the origin of replication is compatible with the first plasmid (Kozlovska, T. M., et al., Intervirology 39:9-15 (1996)). In a third approach, CP and the A1 protein-TNF-peptide fusion are encoded in a bicistronic manner, operatively linked to a promoter such as the Trp promoter, as described in FIG. 1 of Kozlovska et al., Intervirology, 39:9-15 (1996).

In a further embodiment, the TNF-peptide of the invention is inserted between amino acid 2 and 3 (numbering of the cleaved CP, that is wherein the N-terminal methionine is cleaved) of the fr CP, thus leading to a TNF-peptide-fr CP fusion protein. Vectors and expression systems for construction and expression of fr CP fusion proteins self-assembling to VLP and useful in the practice of the invention have been described (Pushko P. et al., Prot. Eng. 6:883-891 (1993)). In a specific embodiment, the sequence of the TNF-peptide of the invention is inserted into a deletion variant of the fr CP after amino acid 2, wherein residues 3 and 4 of the fr CP have been deleted (Pushko P. et al., Prot. Eng. 6:883-891 (1993)).

Fusion of epitopes in the N-terminal protuberant β-hairpin of the coat protein of RNA phage MS-2 and subsequent presentation of the fused epitope on the self-assembled VLP of RNA phage MS-2 has also been described (WO 92/13081), and fusion of the TNF-peptide of the invention by insertion or substitution into the coat protein of MS-2 RNA phage is also falling under the scope of the invention.

In another embodiment of the invention, the TNF-peptides of the invention are fused to a capsid protein of papillomavirus. In a more specific embodiment, the TNF-peptides of the invention are fused to the major capsid protein L1 of bovine papillomavirus type 1 (BPV-1). Vectors and expression systems for construction and expression of BPV-1 fusion proteins in a baculovirus/insect cells systems have been described (Chackerian, B. et al., Proc. Natl. Acad. Sci. USA 96:2373-2378 (1999), WO 00/23955). Substitution of amino acids 130-136 of BPV-1 L1 with a TNF-peptide of the invention leads to a BPV-1 L1-TNF-peptide fusion protein, which is a preferred embodiment of the invention. Cloning in a baculovirus vector and expression in baculovirus infected Sf9 cells has been described, and can be used in the practice of the invention (Chackerian, B. et al., Proc. Natl. Acad. Sci. USA 96:2373-2378 (1999), WO 00/23955). Purification of the assembled particles displaying the fused TNF-peptides of the invention can be performed in a number of ways, such as for example gel filtration or sucrose gradient ultracentrifugation (Chackerian, B. et al., Proc. Natl. Acad. Sci. USA 96:2373-2378 (1999), WO 00/23955).

In a further embodiment of the invention, the TNF-peptides of the invention are fused to a Ty protein capable of being incorporated into a Ty VLP. In a more specific embodiment, the TNF-peptides of the invention are fused to the p1 or capsid protein encoded by the TYA gene (Roth, J. F., Yeast 16:785-795 (2000)). The yeast retrotransposons Ty1, 2, 3 and 4 have been isolated from Saccharomyces Cerevisiae, while the retrotransposon Tf1 has been isolated from Schizosaccharomyces Pombae (Boeke, J. D. and Sandmeyer, S. B., “Yeast Transposable elements,” in The molecular and Cellular Biology of the Yeast Saccharomyces: Genome dynamics, Protein Synthesis, and Energetics., p. 193, Cold Spring Harbor Laboratory Press (1991)). The retrotransposons Ty1 and 2 are related to the copia class of plant and animal elements, while Ty3 belongs to the gypsy family of retrotransposons, which is related to plants and animal retroviruses. In the Ty1 retrotransposon, the p1 protein, also referred to as Gag or capsid protein has a length of 440 amino acids. P1 is cleaved during maturation of the VLP at position 408, leading to the p2 protein, the essential component of the VLP.

Fusion proteins to p1 and vectors for the expression of said fusion proteins in Yeast have been described (Adams, S. E., et al., Nature 329:68-70 (1987)). So, for example, a TNF-peptide of the invention may be fused to p1 by inserting a sequence coding for the TNF-peptide of the invention into the BamH1 site of the pMA5620 plasmid (Adams, S. E., et al., Nature 329:68-70 (1987)). The cloning of sequences coding for foreign epitopes into the pMA5620 vector leads to expression of fusion proteins comprising amino acids 1-381 of p1 of Ty1-15, fused C-terminally to the N-terminus of the foreign epitope. Likewise, N-terminal fusion of TNF-peptides of the invention, or internal insertion into the p1 sequence, or substitution of part of the p1 sequence is also meant to fall within the scope of the invention. In particular, insertion of TNF-peptides of the invention into the Ty sequence between amino acids 30-31, 67-68, 113-114 and 132-133 of the Ty protein p1 (EP0677111) leads to preferred embodiments of the invention.

Further VLPs suitable for fusion of TNF-peptides of the invention are, for example, Retrovirus-like-particles (WO9630523), HIV2 Gag (Kang, Y. C., et al, Biol. Chem. 380:353-364 (1999)), Cowpea Mosaic Virus (Taylor, K. M. et al., Biol. Chem. 380:387-392 (1999)), parvovirus VP2 VLP (Rueda, P. et al., Virology 263:89-99 (1999)), HBsAg (U.S. Pat. No. 4,722,840, EP0020416B1).

Examples of chimeric VLPs suitable for the practice of the invention are also those described in Intervirology 39:1 (1996). Further examples of VLPs contemplated for use in the invention are: HPV-1, HPV-6, HPV-11, HPV-16, HPV-18, HPV-33, HPV-45, CRPV, COPV, HIV GAG, Tobacco Mosaic Virus. Virus-like particles of SV-40, Polyomavirus, Adenovirus, Herpes Simplex Virus, Rotavirus and Norwalk virus have also been made, and chimeric VLPs of those VLPs are also within the scope of the present invention.

TNF-peptides of the invention can be produced by expression of DNA encoding TNF-peptide of the invention under the control of a strong promoter. Various examples hereto have been described in the literature and can be used, possibly after modifications, to express TNF-peptide of the invention of any desired species, preferably in the context of fusion polypeptides, e.g. a fusion with GST or DHFR.

Such TNF-peptides of the invention can be produced using standard molecular biological technologies where the nucleotide sequence coding for the fragment of interest is amplified by PCR and is cloned as a fusion to a polypeptide tag, such as the histdine tag, the Flag tag, myc tag or the constant region of an antibody (Fc region). By introducing an enterokinase cleavage site between the TNF-peptide of the invention and the tag, the TNF-peptide of the invention can be separated from the tag after purification by digestion with enterokinase. In another approach the TNF-peptide of the invention can be synthesized in vitro with or without a phosphorylation-modification using standard peptide synthesis reactions known to a person skilled in the art.

Guidance on how to modify TNF-peptide of the invention, in particular, for binding to the virus-like particle is given throughout the application. Immunization against a member of the TNF-superfamily using the inventive compositions comprising a TNF-peptide of the invention, preferably a human TNF-peptide of the invention, bound to a core particle and VLP, respectively, may provide a way of treating autoimmune diseases and bone-related disorders.

In a still further preferred embodiment of the present invention, the TNF-peptide of the invention further comprises at least one second attachment site not naturally occurring within said TNF-peptide of the invention. In a preferred embodiment, said attachment site comprises an amino acid linker of the invention, preferably a linker sequence of C, CG, GC, GGC or CGG.

Some of the very preferred TNF-peptides of the invention are described in the Examples. These peptides comprise an N- or C-terminal cysteine residue as a second attachment added for coupling to VLPs. These very preferred short TNF-peptides of the invention are capable of having a very enhanced immunogenicity when coupled to VLP and to a core particle, respectively.

In further preferred embodiments of the invention, the TNF-peptide consists of a peptide with a length of 4, 5 or 6 to 8 amino acid residues, preferably with a length of from 4, 5 or 6 or 7 amino acid residues and more preferably with a length of from 4, 5 or 6 to 6 amino acid residues, are, furthermore, capable of overcoming possible safety issues that arise when targeting self-proteins, as shorter fragment are much more less likely to contain T cell epitopes. Typically, the shorter the peptides, the safer with respect to T cell activation.

Further preferred members of the TNF superfamily and TNF-peptides of the invention derived from these molecules may be discovered in the future in species where no sequence information is available yet. The above-mentioned Blastp search explained in the definition of the TNF-superfamily members can help to identify TNF-domains present in these proteins.

The invention further relates to the use of the modified core particle, and in particular the modified VLP, of the invention or of a composition of the invention or of the pharmaceutical composition of the invention for the preparation of a medicament for the treatment of autoimmune-diseases and of bone-related diseases. The treatment is preferably a therapeutic treatment or alternatively a prophylactic treatment. Preferred autoimmune-diseases are rheumatoid arthritis, systemic lupus erythematosis, inflammatory bowel disease, multiple sclerosis, diabetes, autoimmune thyroid disease, autoimmune hepatitis, psoriasis or psoriatic arthritis. Preferred bone related diseases are osteoporosis, periodontis, periprosthetic osteolysis, bone metastasis, bone cancer pain, Paget's disease, multiple myeloma, Sjögren's syndrome and primary billiary cirrhosis.

In a preferred embodiment the TNF-peptide of the modified core particle and preferably of the modified VLP, to be used is derived from a vertebrate polypeptide selected from the group consisting of TNFα, LTα and LTα/β. Such conjugates are preferably to be used for the manufacture of a medicament for the treatment of autoimmune-diseases and of bone-related diseases, preferably of rheumatoid arthritis, systemic lupus erythematosis, inflammatory bowl disease, multiple sclerosis, diabetes, psoriasis, psoriatic arthritis, myasthenia gravis, Sjögren's syndrome and multiple sclerosis, most preferably psoriasis.

In a further preferred embodiment of the invention the TNF-peptide of the modified core particle and in particular of the modified VLP of the invention is derived from a vertebrate, and in particular a eutherian LIGHT polypeptide. Such conjugates are preferably to be used for the manufacture of a medicament for the treatment of autoimmune-diseases and of bone-related diseases, preferably of rheumatoid arthritis and diabetes.

In a further preferred embodiment of the invention the TNF-peptide of the modified core particle and in particular of the modified VLP of the invention is derived from a vertebrate, and in particular a eutherian, FasL polypeptide. Such conjugates are preferably to be used for the manufacture of a medicament for the treatment of autoimmune-diseases and of bone-related diseases, preferably of systemic lupus erhythimatosis, diabetes, autoimmune thyroid disease, autoimmune hepatitis and multiple sclerosis.

In a further preferred embodiment of the invention the TNF-peptide of the modified core particle and in particular of the modified VLP, of the invention is derived from a vertebrate, and in particular a eutherian CD40L polypeptide. Such conjugates are preferably to be used for the manufacture of a medicament for the treatment of autoimmune-diseases and of bone-related diseases, preferably of rheumatoid arthritis, systemic lupus erhythimatosis, inflammatory bowel disease, Sjögren's syndrome and atherosclerosis.

In a further preferred embodiment of the invention the TNF-peptide of the modified core particle and in particular of the modified VLP of the invention is derived from a vertebrate, and in particular a eutherian, TRAIL polypeptide. Such conjugates are preferably to be used for the manufacture of a medicament for the treatment of autoimmune-diseases and of bone-related diseases, preferably of rheumatoid arthritis, multiple sclerosis and autoimmune thyroid disease.

In a further preferred embodiment of the invention the TNF-peptide of the modified core particle and in particular of the modified VLP of the invention is derived from a vertebrate, and in particular a eutherian RANKL polypeptide. Such conjugates are preferably to be used for the manufacture of a medicament for the treatment of autoimmune-diseases and of bone-related diseases, preferably of rheumatoid arthritis, osteoporosis, psoriasis, psoriatic arthritis, multiple myeloma, periodontis, periprosthetic osteolysis, bone metasis, bone cancer pain, periodontal disease and Paget's disease, most preferably psoriasis.

In a further preferred embodiment of the invention the TNF-peptide of the modified core particle and in particular of the modified VLP, of the invention is derived from a vertebrate, and in particular a eutherian CD30L polypeptide. Such conjugates are preferably to be used for the manufacture of a medicament for the treatment of autoimmune-diseases and of bone-related diseases, preferably of rheumatoid arthritis, systemic lupus erythematosis, autoimmune thyroid disease, Sjögren's syndrome, myocarditis and primary billiary cirrhosis.

In a further preferred embodiment of the invention the TNF-peptide of the modified core particle and in particular of the modified VLP, of the invention is derived from a vertebrate, and in particular a eutherian 4-1BBL polypeptide. Such conjugates are preferably to be used for the manufacture of a medicament for the treatment of autoimmune-diseases and of bone-related diseases, inflammatory bowel disease and multiple sclerosis, preferably of rheumatoid arthritis.

In a further preferred embodiment of the invention the TNF-peptide of the modified core particle and in particular of the modified VLP, of the invention is derived from a vertebrate, and in particular a eutherian OX40L polypeptide. Such conjugates are preferably to be used for the manufacture of a medicament for the treatment of autoimmune-diseases and of bone-related diseases, preferably of rheumatoid arthritis, inflammatory bowel disease and multiple sclerosis.

In a further preferred embodiment of the invention the TNF-peptide of the modified core particle and in particular of the modified VLP, of the invention is derived from a vertebrate, and in particular a eutherian BAFF polypeptide. Such conjugates are preferably to be used for the manufacture of a medicament for the treatment of autoimmune-diseases and of bone-related diseases, preferably of systemic lupus erythematosis, rheumatoid arthritis and Sjögren's syndrome.

In a further preferred embodiment of the invention the TNF-peptide of the modified core particle and in particular of the modified VLP, of the invention is derived from a vertebrate, and in particular a eutherian CD27L polypeptide. Such conjugates are preferably to be used for the manufacture of a medicament for the treatment of autoimmune-diseases and of bone-related diseases, preferably of arteriosclerosis and myocarditis.

In a further preferred embodiment of the invention the TNF-peptide of the modified core particle and in particular of the modified VLP, of the invention is derived from a vertebrate, and in particular a eutherian TWEAK polypeptide. Such conjugates are preferably to be used for the manufacture of a medicament for the treatment of autoimmune-diseases and of bone-related diseases, preferably of rheumatoid arthritis, systemic lupus erythematosus and multiple sclerosis.

In a further preferred embodiment of the invention the TNF-peptide of the modified core particle and in particular of the modified VLP, of the invention is derived from a vertebrate, and in particular a eutherian APRIL polypeptide. Such conjugates are preferably to be used for the manufacture of a medicament for the treatment of autoimmune-diseases and of bone-related diseases, preferably of systemic lupus erythematosus, rheumatoid arthritis and Sjögren's syndrome

In a further preferred embodiment of the invention the TNF-peptide of the modified core particle and in particular of the modified VLP, of the invention is derived from a vertebrate, and in particular a eutherian TL1A polypeptide. Such conjugates are preferably to be used for the manufacture of a medicament for the treatment of autoimmune-diseases and of bone-related diseases, preferably of inflammatory bowel disease.

It will be understood by one of ordinary skill in the relevant arts that other suitable modifications and adaptations to the methods and applications described herein are readily apparent and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

Having now fully described the present invention in some detail by way of illustration and example for purposes of clarity of understanding, it will be obvious to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims. All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

### Example 1

### A. Coupling of Murine TNFα(4-23) Peptide to Qβ Capsid Protein

A solution of 3 ml of 3.06 mg/ml Qβ capsid protein in 20 mM HEPES, 150 mM NaCl pH 7.2 was reacted for 60 minutes at room temperature with 99.2 μl of a SMPH solution (65 mM in DMSO). The reaction solution was dialysed at 4° C. against two 3 l changes of 20 mM HEPES, 150 mM NaCl pH 7.2 for 4 hours and 14 hours, respectively. Sixty-nine μl of the derivatized and dialyzed Qβ solution was mixed with 265.5 μl 20 mM HEPES pH 7.2 and 7.5 μl of mTNFα. (4-23) peptide with the second attachment site (SEQ ID NO:29: CGGSSQNSSDKPVAHVVANHQVE) (23.6 mg/ml in DMSO) and incubated for 2 hours at 15° C. for chemical crosslinking. Uncoupled peptide was removed by 2×2 h dialysis at 4° C. against PBS. Coupled products were analysed on a 12% SDS-polyacrylamide gel under reducing conditions. The Coomassie stained gel is shown in FIG. 1. Several bands of increased molecular weight with respect to the Qβ capsid monomer are visible, clearly demonstrating the successful cross-linking of the mTNFα(4-23) peptide to the Qβ capsid.

### B. Immunization of Mice with mTNFα(4-23) Peptide Coupled to Qβ Capsid Protein

Four female Balb/c mice were immunised with Qβ capsid protein coupled to the mTNFα(4-23) peptide. Twenty-five μg of total protein were diluted in PBS to 200 μl and injected subcutaneously (100 μl on two ventral sides) on day 0, day 16 and day 23. Two mice received the vaccine without the addition of any adjuvant while the other two received the vaccine in the presence of Alum. Mice were bled retroorbitally on days 0 and 32, and sera were analysed using mouse TNFα- and human TNFα-specific ELISA.

### C. ELISA

ELISA plates were coated either with mouse TNFα protein or human TNFα protein at a concentration of 1 μg/ml. The plates were blocked and then incubated with serially diluted mouse sera from day 32. Bound antibodies were detected with enzymatically labelled anti-mouse IgG antibody. Antibody titers of mouse sera were calculated as the average of those dilutions which led to half maximal optical density at 450 nm. The average anti-mouse TNFα titers were 18800 for mice which had been immunized in the absence of adjuvant and 16200 for mice which had been immunized in the presence of Alum. Surprisingly, measurement of anti-human TNFα titers of the same sera resulted in strikingly similar values, with averages of 17900 and 12900, respectively. These data demonstrate that immunization with mTNFα(4-23) peptide coupled to Qβ yields antibodies which recognize mouse and human TNFα protein equally well.

### D. Detection of Neutralizing Antibodies

To test whether the antibodies generated in mice have neutralizing activity, in vitro binding assays for both mouse and human TNFα and their cognate receptors mouse TNFRI and human TNFRI were established. ELISA plates were therefore coated with 10 μg/ml of either mouse or human TNFα protein and incubated with serial dilutions of a recombinant mouse TNFRI-hFc fusion protein or a recombinant human TNFRI-hFc fusion protein, respectively. Bound protein was detected with a horse raddish peroxidase conjugated anti-hFc antibody. Both TNFRI/hFc fusion proteins were found to bind with a high affinity (0.1-0.5 nM) to their respective ligands.

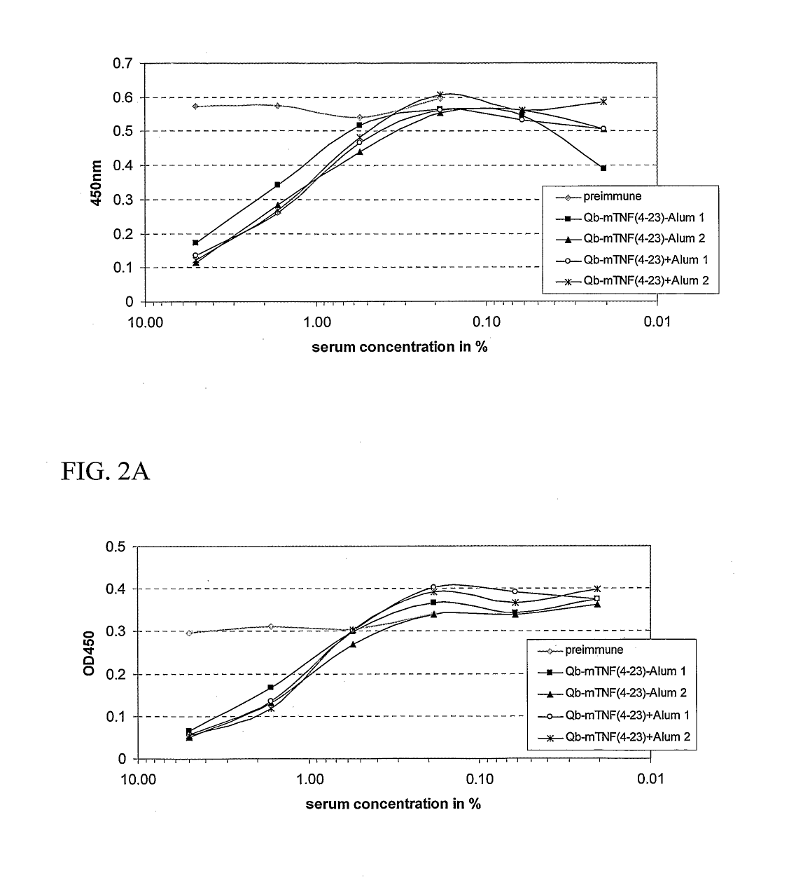

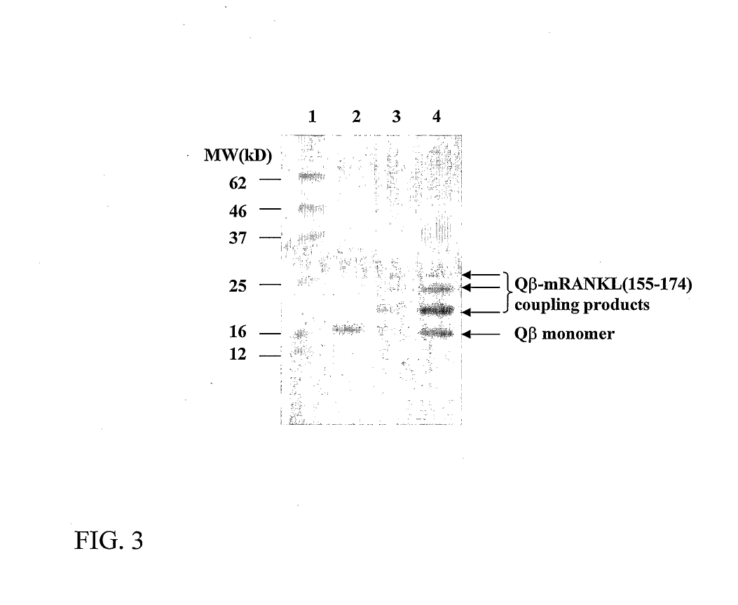

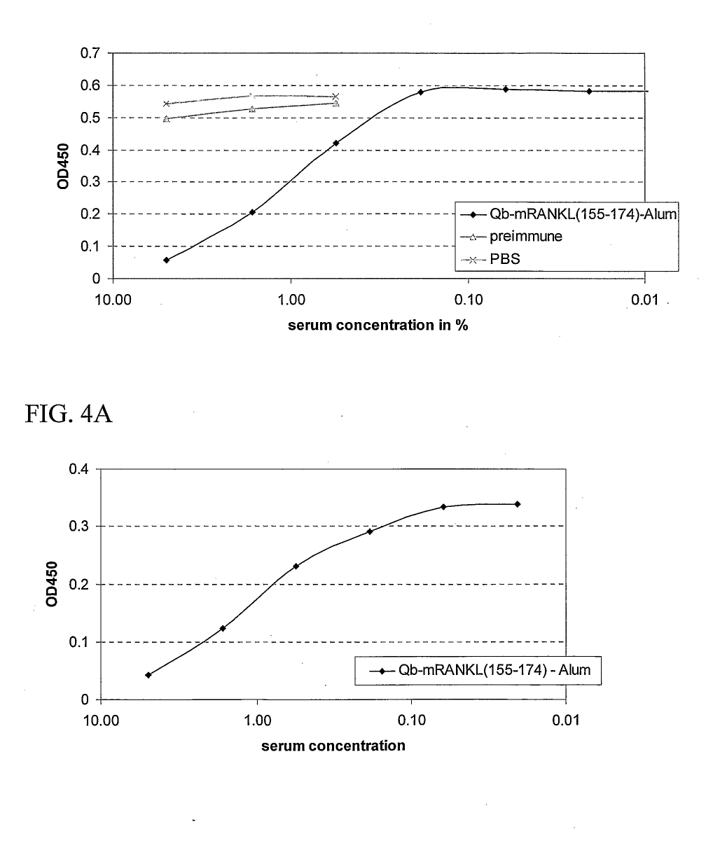

Sera of mice immunized with mTNFα(4-23) coupled to Qβ capsid were then tested for their ability to inhibit the binding of mouse and human TNFα protein to their respective receptors. ELISA plates were therefore coated with either mouse or human TNFα protein at a concentration of 10 μg/ml, and co-incubated with serial dilutions of mouse sera from day 32 and 0.25 nM mouse or human TNFRI-hFc fusion protein, respectively. Binding of receptor to immobilized TNFα protein was detected with horse raddish peroxidase conjugated anti-hFc antibody. FIG. 2A shows that all sera inhibited specifically the binding of mouse TNFα protein to its receptor. Furthermore, as shown in FIG. 2B, the same sera also inhibited the binding of human TNFα protein to its cognate receptor with a similar efficacy. These data demonstrate that immunization with mTNFα(4-23) peptide coupled to Qβ capsid can yield antibodies which are able to neutralize the interactions of both mouse and human TNFα protein with their cognate receptors.

### Example 2

### A. Coupling of mTNFα(11-18) Peptide to Qβ Capsid Protein

A solution of 3.06 mg/ml Qβ capsid protein in 20 mM HEPES, 150 mM NaCl pH 7.2 is reacted for 60 minutes at room temperature with a 10 fold molar excess of SMPH (SMPH stock solution dissolved in DMSO). The reaction solution is dialysed at 4° C. against two 3 l changes of 20 mM HEPES pH 7.2 for 4 hours and 14 hours, respectively. The derivatized and dialyzed Qβ solution is mixed with 20 mM HEPES pH 7.2 and a 5 fold molar excess of mTNFα(11-18) peptide with the second attachment site (SEQ ID NO:2: CGGKPVAHVVA) and incubated for 2 hours at 16° C. for chemical crosslinking. Uncoupled peptide is removed by 2×2 h dialysis at 4° C. against PBS. In case of precipitation, lower excess of SMPH and/or peptide are used. Coupled products are separated on a 12% SDS-polyacrylamide gel under reducing conditions and stained with Coomassie to identify the cross-linking of the mTNFα peptide to the Qβ capsid.

### B. Immunization of Mice with mTNFα(11-18) Peptide Coupled to Qβ Capsid Protein

Eight female Balb/c mice are immunised with Qβ capsid protein coupled to the mTNFα(11-18) peptide. Twenty-five micrograms of total protein are diluted in PBS to 200 μl and injected subcutaneously (100 μl on two ventral sides) on day 0, day 14 and day 21. Four mice receive the vaccine without the addition of any adjuvant and the other 4 mice receive the vaccine in the presence of Alum. Mice are bled retroorbitally on days 0 and 35, and sera are analysed using mouse TNFα protein-specific ELISA.

### C. ELISA

ELISA plates are coated either with mouse TNFα protein at a concentration of 1 μg/ml. The plates are blocked and then incubated with serially diluted pools of mouse sera from day 35. Bound antibodies are detected with enzymatically labelled anti-mouse IgG antibody. Antibody titers of mouse sera are calculated as the average of those dilutions which led to half maximal optical density at 450 nm. Anti-mouse TNFα protein titers are measured to demonstrate the induction of antibodies recognizing the TNFα protein.

### D. Detection of Neutralizing Antibodies

To test whether the antibodies generated in mice have neutralizing activity, in vitro binding assays for mouse TNFα protein and its cognate receptor mouse TNFRI are established. ELISA plates are therefore coated with 10 μg/ml of mouse TNFα protein and incubated with serial dilutions of a recombinant mouse TNFRI-hFc fusion protein. Bound protein is detected with a horse raddish peroxidase conjugated anti-hFc antibody. Sera of mice immunized with mTNFα(11-18) coupled to Qβ capsid are tested for their ability to inhibit the binding of mouse TNFα protein to its receptor. ELISA plates are therefore coated with either mouse TNFα protein at a concentration of 10 μg/ml, and co-incubated with serial dilutions of a pool of mouse sera from day 35 and 0.35 nM mouse fusion protein. Binding of receptor to immobilized TNFα protein and its inhibition by the sera are detected with horse raddish peroxidase conjugated anti-hFc antibody.

### Example 3

### A. Coupling of mTNFα(9-20) Peptide to Qβ Capsid Protein

A solution of 3.06 mg/ml Qβ capsid protein in 20 mM HEPES, 150 mM NaCl pH 7.2 is reacted for 60 minutes at room temperature with a 10 fold molar excess of SMPH (SMPH stock solution dissolved in DMSO). The reaction solution is dialysed at 4° C. against two 3 l changes of 20 mM HEPES pH 7.2 for 4 hours and 14 hours, respectively. The derivatized and dialyzed Qβ solution is mixed with 20 nM HEPES pH 7.2 and a 5 fold molar excess of mTNFα(9-20) peptide with the second attachment site (SEQ ID NO:3: CGGSDKPVAHVVANHQ) and incubated for 2 hours at 16° C. for chemical crosslinking. Uncoupled peptide is removed by 2×2 h dialysis at 4° C. against PBS. In case of precipitation, lower excess of SMPH and/or peptide are used. Coupled products are separated on a 12% SDS-polyacrylamide gel under reducing conditions and stained with Coomassie to identify the cross-linking of the mTNFα peptide to the Qβ capsid.

### B. Immunization of Mice with mTNFα(9-20) Peptide Coupled to Qβ Capsid Protein

Eight female Balb/c mice are immunised with Qβ capsid protein coupled to the mTNFα(9-20) peptide. Twenty-five micrograms of total protein are diluted in PBS to 200 μl and injected subcutaneously (100 μl on two ventral sides) on day 0, day 14 and day 21. Four mice receive the vaccine without the addition of any adjuvant and the other 4 mice receive the vaccine in the presence of Alum. Mice are bled retroorbitally on days 0 and 35, and sera are analysed using mouse TNFα protein-specific ELISA.

### C. ELISA

ELISA plates are coated either with mouse TNFα protein at a concentration of 1 μg/ml. The plates are blocked and then incubated with serially diluted pools of mouse sera from day 35. Bound antibodies are detected with enzymatically labelled anti-mouse IgG antibody. Antibody titers of mouse sera are calculated as the average of those dilutions which led to half maximal optical density at 450 nm. Anti-mouse TNFα protein titers are measured to demonstrate the induction of antibodies recognizing the TNFα protein.

### D. Detection of Neutralizing Antibodies

To test whether the antibodies generated in mice have neutralizing activity, in vitro binding assays for mouse TNFα protein and its cognate receptor mouse TNFRI are established. ELISA plates are therefore coated with 10 μg/ml of mouse TNFα protein and incubated with serial dilutions of a recombinant mouse TNFRI-hFc fusion protein. Bound protein is detected with a horse raddish peroxidase conjugated anti-hFc antibody. Sera of mice immunized with mTNFα(9-20) coupled to Qβ capsid are tested for their ability to inhibit the binding of mouse TNFα protein to its receptor. ELISA plates are therefore coated with either mouse TNFα protein at a concentration of 10 μg/ml, and co-incubated with serial dilutions of a pool of mouse sera from day 35 and 0.35 nM mouse fusion protein. Binding of receptor to immobilized TNFα protein and its inhibition by the sera are detected with horse raddish peroxidase conjugated anti-hFc antibody.

### Example 4

### Efficacy of Qβ-mTNFα(4-23) in Collagen-Induced Arthritis Model

The efficacy of Qβ-mTNFα(4-23) immunization was tested in the murine collagen-induced arthritis (CIA) model. This model reflects most of the immunological and histological aspects of human rheumatoid arthritis and is therefore routinely used to assay the efficacy of anti-inflammatory agents. Male DBA/1 mice were immunized subcutaneously three times (days 0, 14 and 28) with 50 μg of either Qβ-mTNFα(4-23) (n=15) or Qβ alone (n=15), and then injected twice intradermally (days 34 and 55) with 200 μg bovine type II collagen mixed with complete Freund's adjuvant. After the second collagen/CFA injection mice were examined on a regular basis and a clinical score ranging from 0 to 3 was assigned to each limb according to the degree of reddening and swelling observed. Three weeks after the second collagen/CFA injection the average clinical score per limb was 0.04 in the group which had been immunized with Qβ-mTNFα(4-23), and 0.67 in the group which had been immunized with Qβ alone. Moreover, 80% of the mice receiving Qβ-mTNFα(4-23) showed no symptoms at all throughout the course of the experiment, as compared to only 33% of the mice receiving Qβ. We conclude that immunization with Qβ-mTNFα(4-23) protects mice from clinical signs of arthritis in the CIA model.

### Example 5

### A. Coupling of mRANKL(155-174) Peptide to Qβ Capsid Protein

A solution of 3 ml of 3.06 mg/ml Qβ capsid protein in 20 mM HEPES, 150 mM NaCl pH 7.2 was reacted for 60 minutes at room temperature with 25.2 μl of a SMPH solution (65 mM in DMSO). The reaction solution was dialysed at 4° C. against two 3 l changes of 20 mM HEPES pH 7.2 for 4 hours and 14 hours, respectively. Thirty μl of the derivatized and dialyzed Qβ solution was mixed with 167.8 μl 20 mM HEPES pH 7.2 and 2.2 μl of mRANKL(155-174) peptide with the second attachment site (SEQ ID NO:30: CGGQRGKPEAQPFAHLTINAASI) (23.6 mg/ml in DMSO) and incubated for 2 hours at 16° C. for chemical crosslinking. Uncoupled peptide was removed by 2×2 h dialysis at 4° C. against PBS. Coupled products were analysed on a 12% SDS-polyacrylamide gel under reducing conditions. The Coomassie stained gel is shown in FIG. 3. Several bands of increased molecular weight with respect to the Qβ capsid monomer are visible, clearly demonstrating the successful cross-linking of the mRANKL(155-174) peptide to the Qβ capsid.

### B. Immunization of Mice with mRANKL(155-174) Peptide Coupled to Qβ Capsid Protein

Eight female Balb/c mice were immunised with Qβ capsid protein coupled to the mRANKL(155-174) peptide. Twenty-five micrograms of total protein were diluted in PBS to 200 μl and injected subcutaneously (100 μl on two ventral sides) on day 0, day 14 and day 21. Four mice received the vaccine without the addition of any adjuvant and the other 4 mice received the vaccine in the presence of Alum. Mice were bled retroorbitally on days 0 and 35, and sera were analysed using mouse RANKL- and human RANKL-specific ELISA.

### C. ELISA

ELISA plates were coated either with mouse RANKL or human RANKL protein at a concentration of 1 μg/ml. The plates were blocked and then incubated with serially diluted pools of mouse sera from day 35. Bound antibodies were detected with enzymatically labelled anti-mouse IgG antibody. Antibody titers of mouse sera were calculated as the average of those dilutions which led to half maximal optical density at 450 nm. Anti-mouse RANKL titers were 8600 for mice which had been immunized in the absence of adjuvant and 54000 for mice which had been immunized in the presence of Alum. Measurement of anti-human RANKL titers of the same sera resulted in strikingly similar values, with averages of 11200 and 55800, respectively. These data demonstrate that immunization with mRANKL(155-175) peptide coupled to Qβ yields antibodies which recognize mouse and human RANKL protein equally well.

### D. Detection of Neutralizing Antibodies

To test whether the antibodies generated in mice have neutralizing activity, in vitro binding assays for both mouse and human RANKL and their cognate receptors mouse RANK and human RANK were established. ELISA plates were therefore coated with 10 μg/ml of either mouse or human RANKL protein and incubated with serial dilutions of a recombinant mouse RANK-hFc fusion protein or a recombinant human RANK-hFc fusion protein, respectively. Bound protein was detected with a horse raddish peroxidase conjugated anti-hFc antibody. Both RANK-hFc fusion proteins were found to bind with a high affinity (0.1-0.5 nM) to their respective ligands. Sera of mice immunized with mRANKL(155-174) coupled to Qβ capsid were then tested for their ability to inhibit the binding of mouse and human RANKL protein to their respective receptors. ELISA plates were therefore coated with either mouse or human RANKL protein at a concentration of 10 μg/ml, and co-incubated with serial dilutions of a pool of mouse sera from day 35 and 0.35 nM mouse or human RANK-hFc fusion protein, respectively. Binding of receptor to immobilized RANKL protein was detected with horse raddish peroxidase conjugated anti-hFc antibody. FIG. 4A shows that the serum pool inhibited specifically the binding of mouse RANKL protein to its receptor. Furthermore, as shown in FIG. 4B, the same serum pool also inhibited the binding of human RANKL protein to its cognate receptor with a similar efficacy. These data demonstrate that immunization with mRANKL(155-174) peptide coupled to Qβ capsid can yield antibodies which are able to neutralize the interactions of both mouse and human RANKL protein with their cognate receptors.

### Example 6

### A. Coupling of mRANKL(162-170) Peptide to Qβ Capsid Protein

A solution of 3.06 mg/ml Qβ capsid protein in 20 mM HEPES, 150 mM NaCl pH 7.2 is reacted for 60 minutes at room temperature with a 10 fold molar excess of SMPH (SMPH stock solution dissolved in DMSO). The reaction solution is dialysed at 4° C. against two 3 l changes of 20 mM HEPES pH 7.2 for 4 hours and 14 hours, respectively. The derivatized and dialyzed Qβ solution is mixed with 20 mM HEPES pH 7.2 and a 5 fold molar excess of mRANKL(162-170) peptide with the second attachment site (SEQ ID NO:22: CGGQPFAHLTIN) and incubated for 2 hours at 16° C. for chemical crosslinking. Uncoupled peptide is removed by 2×2 h dialysis at 4° C. against PBS. In case of precipitation, lower excess of SMPH and/or peptide are used. Coupled products are separated on a 12% SDS-polyacrylamide gel under reducing conditions and stained with Coomassie to identify the cross-linking of the mRANKL peptide to the Qβ capsid.

### B. Immunization of Mice with mRANKL(162-170) Peptide Coupled to Qβ Capsid Protein

Eight female Balb/c mice are immunised with Qβ capsid protein coupled to the mRANKL(162-170) peptide. Twenty-five micrograms of total protein are diluted in PBS to 200 μl and injected subcutaneously (100 μl on two ventral sides) on day 0, day 14 and day 21. Four mice receive the vaccine without the addition of any adjuvant and the other 4 mice receive the vaccine in the presence of Alum. Mice are bled retroorbitally on days 0 and 35, and sera are analysed using mouse RANKL-specific ELISA.

### C. ELISA

ELISA plates are coated either with mouse RANKL protein at a concentration of 1 μg/ml. The plates are blocked and then incubated with serially diluted pools of mouse sera from day 35. Bound antibodies are detected with enzymatically labelled anti-mouse IgG antibody. Antibody titers of mouse sera are calculated as the average of those dilutions which led to half maximal optical density at 450 nm. Anti-mouse RANKL titers are measured to demonstrate the induction of antibodies recognized the RANKL protein.

### D. Detection of Neutralizing Antibodies

To test whether the antibodies generated in mice have neutralizing activity, in vitro binding assays for mouse RANKL and its cognate receptor mouse RANK are established. ELISA plates are therefore coated with 10 μg/ml of mouse RANKL protein and incubated with serial dilutions of a recombinant mouse RANK-hFc fusion protein. Bound protein is detected with a horse raddish peroxidase conjugated anti-hFc antibody. Sera of mice immunized with mRANKL(162-170) coupled to Qβ capsid are tested for their ability to inhibit the binding of mouse RANKL protein to its receptor. ELISA plates are therefore coated with either mouse RANKL protein at a concentration of 10 μg/ml, and co-incubated with serial dilutions of a pool of mouse sera from day 35 and 0.35 nM mouse fusion protein. Binding of receptor to immobilized RANKL protein and its inhibition by the sera are detected with horse raddish peroxidase conjugated anti-hFc antibody.

### Example 7

### A. Coupling of mRANKL(160-171) Peptide to Qβ Capsid Protein

A solution of 3.06 mg/ml Qβ capsid protein in 20 mM HEPES, 150 mM NaCl pH 7.2 is reacted for 60 minutes at room temperature with a 10 fold molar excess of SMPH (SMPH stock solution dissolved in DMSO). The reaction solution is dialysed at 4° C. against two 3 l changes of 20 mM HEPES pH 7.2 for 4 hours and 14 hours, respectively. The derivatized and dialyzed Qβ solution is mixed with 20 mM HEPES pH 7.2 and a 5 fold molar excess of mRANKL(160-171) peptide with the second attachment site (SEQ ID NO:23: CGGEAQPFAHLTINA) and incubated for 2 hours at 16° C. for chemical crosslinking. Uncoupled peptide is removed by 2×2 h dialysis at 4° C. against PBS. In case of precipitation, lower excess of SMPH and/or peptide are used. Coupled products are separated on a 12% SDS-polyacrylamide gel under reducing conditions and stained with Coomassie to identify the cross-linking of the mRANKL peptide to the Qβ capsid.

### B. Immunization of Mice with mRANKL(160-171) Peptide Coupled to Qβ Capsid Protein

Eight female Balb/c mice are immunised with Qβ capsid protein coupled to the mRANKL(160-171) peptide. Twenty-five micrograms of total protein are diluted in PBS to 200 μl and injected subcutaneously (100 μl on two ventral sides) on day 0, day 14 and day 21. Four mice receive the vaccine without the addition of any adjuvant and the other 4 mice receive the vaccine in the presence of Alum. Mice are bled retroorbitally on days 0 and 35, and sera are analysed using mouse RANKL-specific ELISA.

### C. ELISA

ELISA plates are coated either with mouse RANKL at a concentration of 1 μg/ml. The plates are blocked and then incubated with serially diluted pools of mouse sera from day 35. Bound antibodies are detected with enzymatically labelled anti-mouse IgG antibody. Antibody titers of mouse sera are calculated as the average of those dilutions which led to half maximal optical density at 450 nm. Anti-mouse RANKL titers are measured to demonstrate the induction of antibodies recognized the RANKL protein.

### D. Detection of Neutralizing Antibodies

To test whether the antibodies generated in mice have neutralizing activity, in vitro binding assays for mouse RANKL and its cognate receptor mouse RANK are established. ELISA plates are therefore coated with 10 μg/ml of mouse RANKL protein and incubated with serial dilutions of a recombinant mouse RANK-hFc fusion protein. Bound protein is detected with a horse raddish peroxidase conjugated anti-hFc antibody. Sera of mice immunized with mRANKL(160-171) coupled to Qβ capsid are tested for their ability to inhibit the binding of mouse RANKL protein to its receptor. ELISA plates are therefore coated with either mouse RANKL protein at a concentration of 10 μg/ml, and co-incubated with serial dilutions of a pool of mouse sera from day 35 and 0.35 nM mouse fusion protein. Binding of receptor to immobilized RANKL protein and its inhibition by the sera are detected with horse raddish peroxidase conjugated anti-hFc antibody.

### Example 8

### A. Coupling of mRANKL(161-170) Peptide to Qβ Capsid Protein

A solution of 2.8 mg/ml Qβ capsid protein in 20 mM HEPES, 150 mM NaCl pH 7.2 was reacted for 35 minutes at room temperature with a 20 fold molar excess of SMPH (SMPH stock solution dissolved in DMSO). The reaction solution was dialysed at 4° C. against two 5 l changes of 20 mM HEPES pH 7.4 for a total of 4 hours. The derivatized and dialyzed Qβ solution was mixed with 20 mM HEPES pH 7.4 and a 5 fold molar excess of mRANKL(161-170) peptide with the second attachment site (CGGAQPFAHLTIN, SEQ ID NO:147) and incubated for 2 hours at 15° C. for chemical crosslinking. Uncoupled peptide was removed by overnight dialysis at 4° C. against 5 l of 20 mM HEPES pH 7.4 and an additional dialysis of 2 hours at 4° C. against 3 I of the same buffer. Coupled products were separated on a 12% SDS-polyacrylamide gel under reducing conditions and stained with Coomassie to identify the cross-linking of the mRANKL(161-170) peptide to the Qβ capsid. Several bands of increased molecular weight with respect to the Qβ capsid monomer were visible, clearly demonstrating the successful cross-linking of the mRANKL(161-170) peptide to the Qβ capsid.

### B. Immunization of Mice with Peptide mRANKL(161-170) Coupled to Qβ Capsid Protein

Four female C57B1/6 mice were immunized with Qβ capsid protein coupled to the mRANKL(161-170) peptide. Fifty micrograms of total protein were diluted in PBS to 200 μl and injected subcutaneously (100 μl on two ventral sides) on day 0, 14 and 28. Mice were bled retroorbitally on day 28, and sera were analyzed using mouse RANKL protein-specific ELISA.

### C. ELISA

ELISA plates were coated with mouse RANKL protein at a concentration of 1 μg/ml. The plates were blocked and then incubated with serially diluted mouse sera from day 28. Bound antibodies were detected with enzymatically labeled anti-mouse IgG antibody. Antibody titers of mouse sera were calculated as the average of those dilutions which led to half maximal optical density at 450 nm. The average anti-mouse RANKL titers were 19500, demonstrating that immunization with mRANKL(161-170) peptide coupled to Qβ yielded antibodies which recognize the full-length mRANKL protein.

### D. Detection of Neutralizing Antibodies

Sera of mice immunized with mRANKL(161-170) coupled to Qβ capsid are tested for their ability to inhibit the binding of mouse or human RANKL protein to its respective receptor. ELISA plates are therefore coated with either mouse or human RANKL protein at a concentration of 10 μg/ml, and co-incubated with serial dilutions of a pool of mouse sera from day 35 and 0.35 nM mouse or human mRANK-hFc receptor fusion protein. Binding of receptor to immobilized RANKL protein and its inhibition by the sera are detected with horse raddish peroxidase conjugated anti-hFc antibody.

### Example 9

### A. Coupling of hRANKL(155-174) Peptide to Qβ Capsid Protein

A solution of 2.11 mg/ml Qβ capsid protein in 20 mM HEPES, 150 mM NaCl pH 7.2 was reacted for 1 h at room temperature with a 10 fold molar excess of SMPH (SMPH stock solution dissolved in DMSO). The reaction solution was dialysed over night at 4° C. against 2 l of 20 mM HEPES pH 7.4. The derivatized and dialyzed Qβ solution was mixed with 20 mM HEPES pH 7.4 and a 5 fold molar excess of hRANKL(155-174) peptide with the second attachment site (SEQ ID NO:148, CGGKRSKLEAQPFAHLTINATDI) and incubated for 2 hours at 15° C. for chemical crosslinking. Coupled products were separated on a 12% SDS-polyacrylamide gel under reducing conditions and stained with Coomassie to identify the cross-linking of the hRANKL(155-174) peptide to the Qβ capsid. Several bands of increased molecular weight with respect to the Qβ capsid monomer were visible, clearly demonstrating the successful cross-linking of the hRANKL(155-174) peptide to the Qβ capsid.

### B. Immunization of Mice with Peptide hRANKL(155-174) Coupled to Qβ Capsid Protein

Eight female C57B1/6 mice were immunized with Qβ capsid protein coupled to the hRANKL(155-174) peptide. Twenty-five micrograms of total protein were diluted in PBS to 200 μl and injected subcutaneously (100 μl on two ventral sides) on day 0, day 14 and day 21. Four mice received the vaccine without the addition of any adjuvant and the other 4 mice received the vaccine in the presence of Alum. Mice were bled retroorbitally on day 21, and sera were analyzed using mouse RANKL protein-specific ELISA.

### C. ELISA

ELISA plates were coated with mouse RANKL protein at a concentration of 5 μg/ml. The plates were blocked and then incubated with serially diluted mouse sera from day 21. Bound antibodies were detected with enzymatically labeled anti-mouse IgG antibody. Antibody titers of mouse sera were calculated as the average of those dilutions which led to half maximal optical density at 450 nm. The average anti-mouse RANKL titers were 15000 for mice which had been vaccinated in the absence of Alum, and 23600 for mice which had received the vaccine in the presence of Alum. This demonstrates that immunization with hRANKL(155-174) peptide coupled to Qβ yielded antibodies which recognize the full-length mRANKL protein.

### D. Detection of Neutralizing Antibodies

Sera of mice immunized with hRANKL(155-174) coupled to Qβ capsid are tested for their ability to inhibit the binding of mouse or human RANKL protein to its respective receptor. ELISA plates are therefore coated with either mouse or human RANKL protein at a concentration of 10 μg/ml, and co-incubated with serial dilutions of a pool of mouse sera from day 35 and 0.35 nM mouse or human mRANK-hFc receptor fusion protein. Binding of receptor to immobilized RANKL protein and its inhibition by the sera are detected with horse raddish peroxidase conjugated anti-hFc antibody.

### Example 10

### A. Coupling of mTNFα(10-19) Peptide to Qβ Capsid Protein

A solution of 2.8 mg/ml Qβ capsid protein in 20 mM HEPES, 150 mM NaCl pH 7.2 was reacted for 35 minutes at room temperature with a 20 fold molar excess of SMPH (SMPH stock solution dissolved in DMSO). The reaction solution was dialysed at 4° C. against two 3 l changes of 20 mM HEPES pH 7.4 for a total of 6 hours. The derivatized and dialyzed Qβ solution was mixed with 20 mM HEPES pH 7.4 and a 5 fold molar excess of mTNFα(10-19) peptide with the second attachment site (SEQ ID NO:146, CGGSKPVAHVVAN) and incubated for 2 hours at 15° C. for chemical crosslinking. Uncoupled peptide was removed by 2×2 h dialysis at 4° C. against 20 mM HEPES pH 7.4. Coupled products were separated on a 12% SDS-polyacrylamide gel under reducing conditions and stained with Coomassie to identify the cross-linking of the mTNFα peptide to the Qβ capsid. Several bands of increased molecular weight with respect to the Qβ capsid monomer were visible, clearly demonstrating the successful cross-linking of the mTNFα(10-19) peptide to the Qβ capsid.

### B. Immunization of Mice with mTNFα(10-19) Peptide Coupled to Qβ Capsid Protein

Four female C57B1/6 mice were immunized with Qβ capsid protein coupled to the mTNFα(10-19) peptide. Fifty micrograms of total protein were diluted in PBS to 200 μl and injected subcutaneously (100 μl on two ventral sides) on day 0, 14 and 28. Mice were bled retroorbitally on day 28, and sera were analyzed using mouse or human TNFα protein-specific ELISA.

### C. ELISA

ELISA plates were coated either with mouse or with human TNFα protein at a concentration of 1 μg/ml. The plates were blocked and then incubated with serially diluted mouse sera from day 28. Bound antibodies were detected with enzymatically labeled anti-mouse IgG antibody. Antibody titers of mouse sera were calculated as the average of those dilutions which led to half maximal optical density at 450 nm. The average anti-mouse TNFα titers were 24500, while the average anti-human TNFα titers were 25000. This demonstrates that immunization with mTNFα(10-19) peptide coupled to Qβ yielded antibodies which recognize both human and mouse TNFα protein equally well.

### D. Detection of Neutralizing Antibodies

Sera of mice immunized with mTNFα(10-19) coupled to Qβ capsid are tested for their ability to inhibit the binding of mouse TNFα protein to its receptor. ELISA plates are therefore coated with either mouse TNFα protein at a concentration of 10 μg/ml, and co-incubated with serial dilutions of a pool of mouse sera from day 35 and 0.35 nM recombinant mouse TNFRI-hFc fusion protein. Binding of receptor to immobilized TNFα protein and its inhibition by the sera are detected with horse raddish peroxidase conjugated anti-hFc antibody.

### Example 11

### A. Coupling of Murine (m) CD40L(2-23) Peptide to Qβ Capsid Protein

A solution of 2.78 ml of 2 mg/ml Qβ capsid protein in 20 mM HEPES, 150 mM NaCl pH 7.2 was reacted for 30 minutes at room temperature with 158 μl of a SMPH solution (50 mM in DMSO). The reaction solution was dialyzed at 4° C. against two 3 l changes of phosphate-buffered saline, pH 7.2 for 2 hours and 14 hours, respectively. 2.78 ml of the derivatized and dialyzed Qβ solution was mixed with 925 μl phosphate-buffered saline pH 7.2 and 794 μl of mCD40L(2-23) peptide with a second attachment site (SEQ ID NO:150, CGGQRGDEDPQIAAHVVSEANSN) (23.5 mg/ml in DMSO) and incubated for 2 hours at 15° C. for chemical crosslinking. Uncoupled peptide was removed by three 3 l changes of phosphate-buffered saline, pH 7.2 for 2×2 hours and 1×14 hours at 4° C. Coupled products were analysed on a 12% SDS-polyacrylamide gel under reducing conditions. Several bands of increased molecular weight with respect to the Qβ capsid monomer are visible, clearly demonstrating the successful cross-linking of the mCD40L(2-23) peptide to the Qβ capsid.

### B. Immunization of Mice with mCD40L(2-23) Peptide Coupled to Qβ Capsid Protein

Four female C57BL/6 mice were immunised with Qβ capsid protein coupled to the mCD40L(2-23) peptide. 50 μg of total protein was diluted in PBS to 200 μl and injected subcutaneously (100 μl on two ventral sides) on day 0, day 14 and day 28. Mice were bled retroorbitally on days 0 and 42, and sera were analysed using mouse CD40L-specific ELISA.

### C. ELISA

ELISA plates were coated with mCD40L protein at a concentration of 1 μg/ml. The plates were blocked and then incubated with serially diluted mouse sera from day 42. Bound antibodies were detected with enzymatically labeled anti-mouse IgG antibody. Antibody titers of mouse sera were calculated as the average of those dilutions which led to half maximal optical density at 450 nm. The average anti-mCD40L titer on day 42 was 1287.

### D. Recognition of Soluble mCD40L Protein by Antibodies

To test whether the antibodies generated in mice can bind to soluble recombinant mCD40L, an in vitro inhibition assay for mCD40L was established. Pooled sera from mice immunized with mCD40L(2-23) peptide was incubated, at a 1:1000 dilution, with varying concentrations of soluble recombinant mCD40L, ranging from 0 nM to 150 nM. The mixtures were transferred to ELISA plates coated with 0.5 μg/ml mCD40L protein and bound antibodies were detected with enzymatically labeled anti-mouse IgG antibody. Under these conditions, prior incubation of antibodies with 60 nM soluble mCD40L was sufficient to reduce the subsequent binding of antibodies to plate-bound mCD40L by a factor of two, as measured by the half maximal optical density value at 450 nm. This demonstrates that antibodies from mice immunized with mCD40L(2-23) peptide can bind to both soluble mCD40L and plate-bound mCD40L.

### E. Test for Neutralizing Antibodies

Antibodies from mice immunized with mCD40L(2-23) are used to neutralize B cell proliferation in vitro induced by mouse (m) CD40L/CD40 ligation. B cells are obtained from cell suspensions of mouse lymphoid organs, including spleen and lymph nodes, and can be further purified by magnetic bead separation or by cell sorting using a flow cytometer. B cell proliferation is induced in vitro by standard methods though ligation of B cell mCD40 using a source of mCD40L and survival factors such as murine IL-4. mCD40L is provided, for example, by soluble recombinant mCD40L (Craxton et al (2003) Blood 101, 4464-4471), by recombinantly expressed membrane-bound mCD40L (Hasbold J. et al (1998) Eur. J. Immunol. 28, 1040-1051), by activated murine T cells, or by mCD40L on purified activated murine T cell membranes (Hodgkin P. et al (1996) J. Exp. Med. 184, 277-281). B cell proliferation is measured by standard methods including flow cytometry-based fluorescent dye dilution assays (Lyons A. B. and Parish C. R. (1994) J. Immunol. Methods 171, 131-137) or by the incorporation of radioactive or chemically modified DNA base analogues such as [³H]-thymidine or 5-bromo-2′-deoxyuridine. The presence of neutralizing antibodies against mCD40L is demonstrated by an inhibition of B cell proliferation in the presence of antibodies from mice immunized with mCD40L(2-23) compared to antibodies from mice immunized with Qβ alone or antibodies from unimmunized mice. Antibodies are added to the B cell proliferation culture described above either as whole serum or as the purified IgG fraction isolated from serum by protein G affinity chromatography.

### Example 12

### Coupling of Murine (m) BAFF(36-55) Peptide to Qβ Capsid Protein

A solution of 3 ml of 2 mg/ml Qβ capsid protein in 20 mM HEPES, 150 mM NaCl pH 7.2 was reacted for 30 minutes at room temperature with 171 μl of a SMPH solution (50 mM in DMSO). The reaction solution was dialyzed at 4° C. against three 3 l changes of phosphate-buffered saline, pH 7.2 for 2×2 hours and 1×14 hours, respectively. 3 ml of the derivatized and dialyzed Qβ solution was mixed with 1 ml phosphate-buffered saline pH 7.2 and 214.5 μl of mBAFF(36-55) peptide with the second attachment site (SEQ ID NO:151, CGGNLRNIIQDSLQLIADSDTPT) (24.4 mg/ml in DMSO) and incubated for 2 hours at 15° C. for chemical crosslinking. Uncoupled peptide was removed by three 3 l changes of phosphate-buffered saline, pH 7.2 for 2×2 hours and 1×14 hours at 4° C. Coupled products were analysed on a 12% SDS-polyacrylamide gel under reducing conditions. Several bands of increased molecular weight with respect to the Qβ capsid monomer are visible, clearly demonstrating the successful cross-linking of the mBAFF(36-55) peptide to the Qβ capsid.

### Example 13

### Coupling of Murine (m) LTβ(34-53) Peptide to Qβ Capsid Protein

A solution of 3 ml of 2 mg/ml Qβ capsid protein in 20 mM HEPES, 150 mM NaCl pH 7.2 was reacted for 30 minutes at room temperature with 85.8 μl of a SMPH solution (50 mM in DMSO). The reaction solution was dialyzed at 4° C. against three 3 l changes of 20 mM HEPES, pH 7.2 for 2 hours each. 3 ml of the derivatized and dialyzed Qβ solution was mixed with 993 μl 20 mM HEPES pH 7.2 and 429 μl of mLTβ(34-53) peptide with the second attachment site (SEQ ID NO:152, CGGETDLNPELPAAHLIGAWMSG) (23.4 mg/ml in DMSO) and incubated for 2 hours at 15° C. for chemical crosslinking. Uncoupled peptide was removed by three 3 l changes of 20 mM HEPES pH 7.2 for 2×2 hours and 1×14 hours at 4° C. Coupled products were analysed on a 12% SDS-polyacrylamide gel under reducing conditions. Several bands of increased molecular weight with respect to the Qβ capsid monomer are visible, clearly demonstrating the successful cross-linking of the mLTβ(34-53) peptide to the Qβ capsid.

### Example 14

### Binding of Human TNFα to its Receptor hTNF-RI can be Inhibited with Sera from Human Subjects Immunized with mTNF(4-23)Qβ

Human volunteers are immunized with 100 μg mTNF(4-23)Qβ subcutaneously. 28 days later a second immunization using the same dose is performed. Anti-TNFα-specific antibody levels are analysed by ELISA of sera taken two weeks after the final immunization. ELISA plates (Maxisorp, Nunc) are coated with hTNFα (Peprotech) (1 μg/ml) overnight and blocked with the blocking agent Superblock (Pierce). After washing, plates are incubated with eight dilutions of study sera for 2 h. After a further washing step, the secondary anti-human IgG horse-radish peroxidase conjugate (Jackson ImmunoResearch) is added for 1 h. Bound enzyme is detected by reaction with o-phenylenediamine (OPD, Fluka) for 4.5 min and was stopped by addition of sulfuric acid. Optical densities are read in the ELISA reader at 492 nm. The ELISA shows that vaccination of human subjects with mouse TNF(4-23)Qβ induced antibodies which bind to human TNFα. The assay described in Example 1 is used to show that the binding of human TNFα to its receptor hTNF-RI can be inhibited with sera from subjects immunized with mTNF(4-23)Qβ further supporting the cross-reactivity of antibodies induced by vaccination against mTNF(4-23) to human TNFα protein.

### Example 15

### Treatment of Psoriasis with mTNF(4-23)Qβ

Patients suffering from moderate to severe plaque psoriasis are immunized with 100 μg or 300 μg mTNF(4-23)Qβ at days 0 and day 28. A further boosting immunization is given at day 84. Clinical efficacy will be assessed using the psoriasis area and severity index (PASI) and the physician global assessment (PGA) criteria. Clinical scores are taken at baseline and at biweekly intervals. Because of the expected variability in antibody titers, the evaluation of clinical efficacy of vaccination will discriminate the magnitude of the response (PASI score or PGA score) by the degree of antibody response. Evaluations will be done using antibody titers as a covariate or by stratification of patients according to their antibody response. The results show that vaccination with mTNF(4-23)Qβ results in reduced clinical scores in plaque psoriasis patients.

1. A modified virus like particle (VLP) comprising:
a) a virus like particle (VLP), and
b) at least one peptide (TNF-peptide) comprising a peptide sequence homologous to amino acid residues 3 to 8 of the consensus sequence for the conserved domain pfam 00229 (SEQ ID NO:1), preferably a peptide sequence homologous to amino acid residues 1 to 8 of the consensus sequence for the conserved domain pfam 00229 (SEQ ID NO:1),
wherein a) and b) are linked with one another, and wherein said TNF-peptide consists of a peptide with a length of 6 to 18 amino acid residues, preferably with a length of 6 to 16 amino acid residues, more preferably with a length of 6 to 14 amino acid residues, when the TNF-peptide is a peptide from human or mouse TNFα, and wherein TNF-peptide consists of a peptide with a length of 6 to 50 amino acid residues, preferably with a length of 6 to 40 amino acid residues, more preferably with a length of 6 to 30 amino acid residues, when the TNF-peptide is a peptide from human or mouse RANKL, from human or mouse LTα, or from human or mouse LTβ.

2. The modified VLP of claim 1, wherein said TNF-peptide consists of a peptide with a length of 4 to 50 amino acid residues, preferably with a length of from 6 to 40 amino acid residues, more preferably with a length of from 6 to 30 amino acid residues, even more preferably with a length of from 6 to 20 amino acid residues, again even more preferably with a length of from 6 to 18 amino acid residues and even more preferred with a length of from 6 to 16 amino acid residues.

3. The modified VLP of any one of claims 1 or 2, wherein said TNF-peptide is derived from a vertebrate, preferably a mammalian, polypeptide selected from the group consisting of TNFα, LTα, LTα/β, FasL, CD40L, TRAIL, RANKL, CD30L, 4-1BBL, OX40L, LIGHT, GITRL and BAFF, CD27L, TWEAK, APRIL, TL1A, EDA, preferably selected from the group consisting of TNFα, LTα and LTα/β, or selected from the group consisting of TRAIL and RANKL, or selected from the group consisting of FasL, CD40L, CD30L and BAFF, or selected from the group consisting of 4-1BBL, OX40L and LIGHT, or selected from the group consisting of LTα, LTα/β, Fasl, CD40L, TRAIL, CD30L, 4-1BBL, OX40L, GITRL and BAFF.

4. The modified VLP of any one of claims 1 to 3, wherein said modified VLP forms an ordered and repetitive antigen array.

5. The modified VLP of any one of claims 1 to 4, wherein said VLP (a) and said TNF-peptide (b) are covalently linked.

6. The modified VLP of any one of claims 1 to 5, wherein said VLP comprises, or alternatively consists of, recombinant proteins, or fragments thereof, of a RNA-phage, and wherein preferably said RNA-phage is RNA-phage Qβ, RNA-phage fr or RNA-phage AP205, and wherein further preferably said RNA-phage is RNA-phage Qβ.

7. The modified VLP of claim 6, wherein said recombinant proteins comprise, or alternatively consist essentially of, or alternatively consist of coat proteins of RNA phages, and wherein preferably said coat proteins of RNA phages having an amino acid are selected from the group consisting of
(a) SEQ ID NO:4;
(b) a mixture of SEQ ID NO:4 and SEQ ID NO:5;
(c) SEQ ID NO:6;
(d) SEQ ID NO:7;
(e) SEQ ID NO:8;
(f) SEQ ID NO:9;
(g) a mixture of SEQ ID NO:9 and SEQ ID NO:10;
(h) SEQ ID NO:11;
(i) SEQ ID NO:12;
(k) SEQ ID NO:13;
(l) SEQ ID NO:14;
(m) SEQ ID NO:15;
(n) SEQ ID NO:16; and
(O) SEQ ID NO:28.

8. The modified VLP of any one of claims 1 to 6 wherein the recombinant proteins comprise, or alternatively consist essentially of, or alternatively consist of mutant coat proteins of RNA phages, and wherein said RNA-phage is selected from the group consisting of:
(a) bacteriophage Qβ;
(b) bacteriophage R17;
(c) bacteriophage fr;
(d) bacteriophage GA;
(e) bacteriophage SP;
(f) bacteriophage MS2;
(g) bacteriophage M11;
(h) bacteriophage MX1;
(i) bacteriophage NL95;
(k) bacteriophage f2;
(l) bacteriophage PP7; and
(m) bacteriophage AP205.

9. The modified VLP of claim 8, wherein said mutant coat proteins of said RNA phage have been modified by (i) removal of at least one lysine residue by way of substitution; (ii) addition of at least one lysine residue by way of substitution; (iii) deletion of at least one lysine residue; and/or (iv) addition of at least one lysine residue by way of insertion.

10. The modified VLP of any one of the preceding claims, wherein the VLP (a) is linked with the TNF-peptide (b) through at least one non-peptide bond.

11. The modified VLP of any one of the claims 1 to 9, wherein said TNF-peptide is fused to said VLP, and wherein preferably said TNF-peptide is fused via its C-terminus to the VLP, or alternatively via its N-terminus.

12. The modified VLP of any one of the preceding claims further comprising an amino acid linker (c) between the VLP (a) and the TNF-peptide (b), wherein (c) and (b) together do not form a peptide having a sequence from human or mouse TNFα, and wherein preferably said amino acid linker is selected from the group consisting of:
(a) GGC;
(b) GGC-CONH2;
(c) GC;
(d) GC-CONH2;
(e) C; and
(f) C-CONH2.

13. The modified VLP of any one of the preceding claims, wherein said modified VLP comprises said VLP with at least one first attachment site, and wherein said modified VLP comprises said TNF peptide with at least one second attachment site, and wherein said second attachment site is capable of association to said first attachment site; and wherein preferably said TNF peptide and VLP interact through said association to form an ordered and repetitive antigen array.

14. The modified VLP of claim 13, wherein said first attachment site comprises, or preferably is, an amino group, and wherein even further preferably said first attachment site is an amino group of a lysine residue.

15. The modified VLP of any of claims 13 to 14, wherein said second attachment site comprises, or preferably is, a sulfhydryl group, and wherein even further preferably said second attachment site is a sulfhydryl group of a cysteine residue.

16. The modified VLP of any of claims 13 to 15, wherein said first attachment site is not, and preferably does not comprise, a sulfhydryl group, and wherein further preferably said first attachment site is not, and again preferably does not comprise, a sulfhydryl group of a cysteine residue.

17. A composition comprising a modified VLP of any one of claims 1 to 16.

18. A pharmaceutical composition comprising:
(a) the modified VLP of any one of claims 1 to 16; and
(b) a pharmaceutically acceptable carrier;
and wherein preferably said pharmaceutical composition (i) further comprises an adjuvant, or (ii) is devoid of an adjuvant.

19. A vaccine composition comprising a modified VLP of any one of claims 1 to 16; and wherein preferably said vaccine composition (i) further comprises an adjuvant, or (ii) is devoid of an adjuvant, and wherein further preferably said modified VLP comprises recombinant proteins or fragments thereof, of RNA-phage Qβ.

20. The vaccine composition of claims 19, wherein said TNF-peptide is derived from a polypeptide selected from the group consisting of:
(a) human TNFα;
(b) human LTα;
(c) human LTα/β;
(d) human FasL;
(e) human CD40L;
(f) human TRAIL;
(g) human RANKL;
(h) human CD30L;
(i) human 4-1BBL;
(j) human OX40L;
(k) human GITRL;
(l) human BAFF;
(m) human LIGHT;
(n) human CD27L;
(O) human TWEAK;
(p) human APRIL;
(q) human TL1A; and
(r) human EDA.

21. Modified VLP of any one of claims 1 to 16 or composition of claim 17 for use as a medicament.

22. (canceled)

23. (canceled)

24. (canceled)

25. A method of immunization comprising administering the modified VLP of claim 1, the composition of claim 17, the pharmaceutical composition of claim 18, or the vaccine composition of claim 19 to an animal or human, preferably a human.

26. A method of treating an autoimmune disease or a bone related disease by administering to a subject, preferably to a human, the modified VLP of claim 1, the composition of claim 17, the pharmaceutical composition of claim 18 or the vaccine composition of claim 19, wherein the autoimmune disease or the bone related disease is selected from the group consisting of (a) psoriasis; (b) rheumatoid arthritis; (c) multiple sclerosis; (d) diabetes; (e) osteoporosis; (f) ankylosing spondylitis; (g) atherosclerosis; (h) autoimmune hepatitis; (i) autoimmune thyroid disease; (i) bone cancer pain; (k) bone metastasis; (l) inflammatory bowel disease; (m) multiple myeloma; (n) myasthenia gravis; (O) myocarditis; (p) Paget's disease; (q) periodontal disease; (r) periodontitis; (s) periprosthetic osteolysis; (t) polymyositis; (u) primary biliary cirrhosis; (v) psoriatic arthritis; (w) Sjögren's syndrome; (x) Still's disease; (y) systemic lupus erythematosus; and (z) vasculitis.
